SEQUENCE LISTING (55 sequences)

SEQ ID NO: 1 - DNA, 79 bp, synthetic construct
  source 1..79: mol_type = other DNA
  misc_feature 1: note = 5' terminal phosphate group
nnngttcagagttctacagtccgacgatcnnnnnnnnnnnnnnnnccaggaatagttatgtgcattaatgaatggccgc

SEQ ID NO: 2 - DNA, 76 bp, synthetic construct
  source 1..76: mol_type = other DNA
  misc_feature 1: note = 5' terminal phosphate group
nnncctacacgacgctcttccgatctnnnnnnnnnnnnnnnnacaggaatagttatgtgcattaatgaatggccgc

SEQ ID NO: 3 - DNA, 89 bp, synthetic construct
  source 1..89: mol_type = other DNA
  misc_feature 1: note = 5' terminal phosphate group
nnncctacacgacgctcttccgatctnnnnnnnnnnnnnnnnacaattcctatcgttcacgtcgtgtcgccatttagtgtccagtctga

SEQ ID NO: 4 - DNA, 76 bp, synthetic construct
  source 1..76: mol_type = other DNA
  misc_feature 1: note = 5' terminal phosphate group
nnncctacacgacgctcttccgatctnnnnnnnnnnnnnnnnccaggaatagttatgtgcattaatgaatggcgcc

SEQ ID NO: 5 - DNA, 30 bp, synthetic construct
  source 1..30: mol_type = other DNA
  modified_base 9: mod_base = OTHER; note = deoxyuracil
  modified_base 18: mod_base = OTHER; note = deoxyuracil
  modified_base 28: mod_base = OTHER; note = 3' deoxyuracil
  modified_base 29^30: mod_base = OTHER; note = phosphorothioate linkage
ccattcatnaatgcacanaactattccngg

SEQ ID NO: 6 - DNA, 29 bp, synthetic construct
  source 1..29: mol_type = other DNA
  modified_base 9: mod_base = OTHER; note = deoxyuracil
  modified_base 18: mod_base = OTHER; note = deoxyuracil
  modified_base 28: mod_base = OTHER; note = deoxyuracil
ccattcatnaatgcacanaactattccng

SEQ ID NO: 7 - DNA, 25 bp, synthetic construct
  source 1..25: mol_type = other DNA
  modified_base 9: mod_base = OTHER; note = deoxyuracil
  modified_base 16: mod_base = OTHER; note = deoxyuracil
  modified_base 23: mod_base = OTHER; note = deoxyuracil
  modified_base 24^25: mod_base = OTHER; note = phosphorothioate internucleotide linkage
acacgacgngaacganaggaatngt

SEQ ID NO: 8 - DNA, 13 bp, synthetic construct
  source 1..13: mol_type = other DNA
  modified_base 12^13: mod_base = OTHER; note = phosphorothioate internucleotide linkage
ccgagaattccat

SEQ ID NO: 9 - DNA, 21 bp, synthetic construct
  source 1..21: mol_type = other DNA
  misc_feature 1: note = 5' terminal phosphate group
tggaattctcgggtgccaagg

SEQ ID NO: 10 - DNA, 63 bp, synthetic construct
  source 1..63: mol_type = other DNA
  misc_feature 25..30: note = 6-base Illumina TruSeq Small RNA multiplexing index sequence
  misc_feature 1..24: note = DNA segment
  misc_feature 31..63: note = DNA segment
caagcagaagacggcatacgagatnnnnnngtgactggagttccttggcacccgagaattcca

SEQ ID NO: 11 - DNA, 58 bp, synthetic construct
  source 1..58: mol_type = other DNA
  modified_base 57^58: mod_base = OTHER; note = phosphorothioate internucleotide linkage
aatgatacggcgaccaccgagatctacactctttccctacacgacgctcttccgatct

SEQ ID NO: 12 - DNA, 28 bp, synthetic construct
  source 1..28: mol_type = other DNA
acactctttccctacacgacgctcttcc

SEQ ID NO: 13 - DNA, 19 bp, synthetic construct
  source 1..19: mol_type = other DNA
  misc_feature 1: note = 5' terminal phosphate group
  modified_base 1^2: mod_base = OTHER; note = phosphorothioate internucleotide linkage
atcggaagagcacacgtct

SEQ ID NO: 14 - DNA, 64 bp, synthetic construct
  source 1..64: mol_type = other DNA
  misc_feature 25..30: note = 6-base Illumina TruSeq Small RNA multiplexing index sequence
  modified_base 63^64: mod_base = OTHER; note = phosphorothioate internucleotide linkage
  misc_feature 1..24: note = DNA segment
  misc_feature 31..64: note = DNA segment
caagcagaagacggcatacgagatnnnnnngtgactggagttcagacgtgtgctcttccgatct

SEQ ID NO: 15 - DNA, 50 bp, synthetic construct
  source 1..50: mol_type = other DNA
aatgatacggcgaccaccgagatctacacgttcagagttctacagtccga

SEQ ID NO: 16 - DNA, 90 bp, synthetic construct
  source 1..90: mol_type = other DNA
  misc_feature 25..30: note = 6-base Illumina TruSeq Small RNA multiplexing index sequence
  misc_feature 1..24: note = DNA segment
  misc_feature 31..90: note = DNA segment
caagcagaagacggcatacgagatnnnnnngtgactggagttcagacgtgtgctcttccgatcccattcattaatgcacataactattcc

SEQ ID NO: 17 - DNA, 104 bp, synthetic construct
  source 1..104: mol_type = other DNA
ccattcattaatgcacataactattcctggnnnnnnnnnnnnnnnngatcgtcggactgtagaactctgaacttttttttttttttttttttttttttttttvn

SEQ ID NO: 18 - DNA, 77 bp, synthetic construct
  source 1..77: mol_type = other DNA
  misc_feature 1..74: note = DNA segment
  misc_feature 75..77: note = RNA bases
  modified_base 77: mod_base = OTHER; note = Locked nucleid acid - G base with 2'-O,4'-C-methylene linker or bridge
gcggccattcattaatgcacataactattcctgtnnnnnnnnnnnnnnnnagatcggaagagcgtcgtgtaggtnnn

SEQ ID NO: 19 - DNA, 23 bp, synthetic construct
  source 1..23: mol_type = other DNA
  misc_feature 1: note = 5' probe fluorophore conjugation
  misc_feature 9: note = ZEN probe quencher conjugation
  misc_feature 23: note = 3IABkFQ probe quencher conjugation
cctacacgacgctcttccgatct

SEQ ID NO: 20 - DNA, 81 bp, synthetic construct
  source 1..81: mol_type = other DNA
  misc_feature 53..58: note = 6-base Illumina TruSeq Small RNA multiplexing index sequence
  misc_feature 1..52: note = DNA segment
  misc_feature 59..81: note = DNA segmant
nnncctacacgacgctcttccgatctnnnnnnnnnnnnnnnnnnnnnncaggaatagttatgtgcattaatgaatggcgcc

SEQ ID NO: 21 - DNA, 25 bp, synthetic construct
  source 1..25: mol_type = other DNA
catgtaatgcacgtactttcagggt

SEQ ID NO: 22 - DNA, 35 bp, synthetic construct
  source 1..35: mol_type = other DNA
cgtgctggattggctcaccagacaccttccgacat

SEQ ID NO: 23 - DNA, 35 bp, synthetic construct
  source 1..35: mol_type = other DNA
atgtcggaaggtgtgcaggctaccgcttgtcaact

SEQ ID NO: 24 - DNA, 23 bp, synthetic construct
  source 1..23: mol_type = other DNA
agtcgtcgcagcctcacctgatc

SEQ ID NO: 25 - DNA, 25 bp, synthetic construct
  source 1..25: mol_type = other DNA
accctgaaagtacgtgcattacatg

SEQ ID NO: 26 - DNA, 23 bp, synthetic construct
  source 1..23: mol_type = other DNA
gatcaggtgaggctgcgacgact

SEQ ID NO: 27 - DNA, 48 bp, synthetic construct
  source 1..48: mol_type = other DNA
accctgaaagtacgtgcattacatggatcaggtgaggctgcgacgact

SEQ ID NO: 28 - DNA, 23 bp, synthetic construct
  source 1..23: mol_type = other DNA
gatcaggtgaggctgcgacgact

SEQ ID NO: 29 - DNA, 25 bp, synthetic construct
  source 1..25: mol_type = other DNA
catgtaatgcacgtactttcagggt

SEQ ID NO: 30 - DNA, 20 bp, synthetic construct
  source 1..20: mol_type = other DNA
aatgatacggcgaccaccga

SEQ ID NO: 31 - DNA, 33 bp, synthetic construct
  source 1..33: mol_type = other DNA
acactctttccctacacgacgctcttccgatct

SEQ ID NO: 32 - DNA, 33 bp, synthetic construct
  source 1..33: mol_type = other DNA
tcgtcggcagcgtcagatgtgtataagagacag

SEQ ID NO: 33 - DNA, 34 bp, synthetic construct
  source 1..34: mol_type = other DNA
agatcggaagagcacacgtctgaactccagtcac

SEQ ID NO: 34 - DNA, 34 bp, synthetic construct
  source 1..34: mol_type = other DNA
ctgtctcttatacacatctccgagcccacgagac

SEQ ID NO: 35 - skipped

SEQ ID NO: 36 - DNA, 20 bp, synthetic construct
  source 1..20: mol_type = other DNA
tcggtggtcgccgtatcatt

SEQ ID NO: 37 - DNA, 24 bp, synthetic construct
  source 1..24: mol_type = other DNA
aatgatacggcgaccaccgagatc

SEQ ID NO: 38 - DNA, 21 bp, synthetic construct
  source 1..21: mol_type = other DNA
caagcagaagacggcatacga

SEQ ID NO: 39 - DNA, 24 bp, synthetic construct
  source 1..24: mol_type = other DNA
caagcagaagacggcatacgagat

SEQ ID NO: 40 - DNA, 25 bp, synthetic construct
  source 1..25: mol_type = other DNA
accctgaaagtacgtgcattacatg

SEQ ID NO: 41 - DNA, 23 bp, synthetic construct
  source 1..23: mol_type = other DNA
gatcaggtgaggctgcgacgact

SEQ ID NO: 42 - DNA, 89 bp, synthetic construct
  source 1..89: mol_type = other DNA
tcggtggtcgccgtatcattaccctgaaagtacgtgcattacatggatcaggtgaggctgcgacgactcaagcagaagacggcatacga

SEQ ID NO: 43 - DNA, 25 bp, synthetic construct
  source 1..25: mol_type = other DNA
catgtaatgcacgtactttcagggt

SEQ ID NO: 44 - DNA, 48 bp, synthetic construct
  source 1..48: mol_type = other DNA
agtcgtcgcagcctcacctgatccatgtaatgcacgtactttcagggt

SEQ ID NO: 45 - DNA, 48 bp, synthetic construct; identical to SEQ ID NO: 44
  source 1..48: mol_type = other DNA

SEQ ID NO: 46 - DNA, 76 bp, synthetic construct; identical to SEQ ID NO: 4
  source 1..76: mol_type = other DNA

SEQ ID NO: 47 - DNA, 44 bp, synthetic construct
  source 1..44: mol_type = other DNA
nnncctacacgacgctcttccgatctnnnnnnnnnnnnnnnncc

SEQ ID NO: 48 - DNA, 88 bp, synthetic construct
  source 1..88: mol_type = other DNA
nnncctacacgacgctcttccgatctnnnnnnnnnnnnnnnnccggnnnnnnnnnnnnnnnnagatcggaagagcgtcgtgtaggnnn

SEQ ID NO: 49 - DNA, 81 bp, synthetic construct
  source 1..81: mol_type = other DNA
nnncctacacgacgctcttccgatctnnnnnnnnnnnnnnnnatcacgcaggaatagttatgtgcattaatgaatggcgcc

SEQ ID NO: 50 - DNA, 49 bp, synthetic construct
  source 1..49: mol_type = other DNA
nnncctacacgacgctcttccgatctnnnnnnnnnnnnnnnnatcacgc

SEQ ID NO: 51 - DNA, 46 bp, synthetic construct
  source 1..46: mol_type = other DNA
cctacacgacgctcttccgatctnnnnnnnnnnnnnnnnatcacgc

SEQ ID NO: 52 - DNA, 41 bp, synthetic construct
  source 1..41: mol_type = other DNA
cctacacgacgctcttccgatctnnnnnnnnnnnnnnnncc

SEQ ID NO: 53 - DNA, 49 bp, synthetic construct; identical to SEQ ID NO: 50
  source 1..49: mol_type = other DNA

SEQ ID NO: 54 - DNA, 28 bp, synthetic construct
  source 1..28: mol_type = other DNA
aggaatagttatgtgcattaatgaatgg

SEQ ID NO: 55 - DNA, 23 bp, synthetic construct
  source 1..23: mol_type = other DNA
cctacacgacgctcttccgatct

(19) United States
(12) Patent Application Publication
(10) Pub. No.: US 2023/0392201 A1
(43) Pub. Date: Dec. 7, 2023

(54) METHODS FOR ASSEMBLING AND READING NUCLEIC ACID SEQUENCES FROM MIXED POPULATIONS

(71) Applicant: Element Biosciences, Inc., San Diego, CA (US)

(72) Inventors: James A. STAPLETON, San Diego, CA (US); Timothy WHITEHEAD, San Diego, CA (US); Michael PREVITE, San Diego, CA (US); Molly HE, San Diego, CA (US); Tuval BEN-YEHEZKEL, San Diego, CA (US); Matthew KELLINGER, San Diego, CA (US); Kyle METCALFE, San Diego, CA (US)

(21) Appl. No.: 18/330,279

(22) Filed: Jun. 6, 2023

Related U.S. Application Data
(60) Provisional application No. 63/349,548, filed on Jun. 6, 2022.

(51) Int. Cl. C12Q 1/6869; C12Q 1/686; C12N 15/10; C12Q 1/6813

(52) U.S. Cl. CPC C12Q 1/6869; C12Q 1/686; C12N 15/1065; C12Q 1/6813

(57) ABSTRACT

The disclosure relates to methods for obtaining nucleic acid sequence information by constructing a nucleic acid library and reconstructing longer nucleic acid sequences by assembling a series of shorter nucleic acid sequences.

### CROSS-REFERENCE TO RELATED APPLICATIONS

This application claims the benefit of and priority to U.S. Provisional Patent Application No. 63/349,548, filed Jun. 6, 2022, the contents of which is incorporated by reference herein in its entirety.

### STATEMENT OF GOVERNMENT INTEREST

This invention was made with government support under GM099291 awarded by the National Institutes of Health. The government has certain rights in the invention.

### REFERENCE TO AN ELECTRONIC SEQUENCE LISTING

The contents of the electronic sequence listing (ELEM_012_001US_SeqList_ST26.xml; Size 68,026 bytes; and Date of Creation: Aug. 14, 2023) are herein incorporated by reference in their entirety.

### TECHNICAL FIELD

The present disclosure provides methods for obtaining nucleic acid sequence information by constructing a nucleic acid library and reconstructing longer nucleic acid sequences by assembling a series of shorter nucleic acid sequences.

### BACKGROUND

The transition from traditional Sanger-style sequencing methods to next-generation sequencing methods has lowered the cost of sequencing, yet significant limitations of next-generation sequencing methods remain. In one respect, available sequencing platforms generate sequencing reads that, while numerous, are relatively short and can require computational reassembly into full sequences of interest. Available assembly methods can be slow, laborious, expensive, computationally demanding, and/or unsuitable for populations of similar individuals (e.g., viruses). This is especially true for sequencing of complex genomes. Assembly is challenging, in part due to the ever-swelling sequencing datasets associated with assembly of short reads. Such datasets can place a large strain on computer clusters. For example, de novo assembly can require that sequencing reads (or k-mers derived from them) be stored in random access memory (RAM) simultaneously. For large datasets this requirement is not trivial. Moreover, even when assembly is possible, crucial haplotype information often cannot be recovered. Indeed, inherent limitations of available technologies obstruct improvements to overcoming the shortcomings of status quo sequencing technologies. Thus, there exists a need for improved sequencing methods and associated assembly techniques that reduce the time and/or computational requirements necessary to obtain accurate sequences.

### SUMMARY

In one aspect, provided herein is a method for obtaining nucleic acid sequence information from a nucleic acid molecule comprising a target nucleotide sequence by assembling a series of nucleic acid sequences into a longer nucleic acid sequence, said method comprising: (a) attaching a first adapter at the 5′ end and/or the 3′ end of a linear nucleic acid molecule, said first adapter comprising an outer polymerase chain reaction (PCR) primer region or nucleic acid amplification region, an inner sequencing primer region, and a central barcode region to each end of a plurality of linear nucleic acid molecules to form barcode-tagged molecules;
(b) replicating the barcode-tagged molecules to obtain a library of barcode-tagged molecules;
(c) breaking the library of barcode-tagged molecules, thereby generating a first set of linear, barcode-tagged fragments, each comprising the barcode region at one end and a region of unknown sequence at the other end;
(d) circularizing the first set of linear, barcode-tagged fragments comprising the barcode region at one end and a region of unknown sequence from an interior portion of the target nucleotide sequence at the other end, thereby bringing the barcode region into proximity with the region of unknown sequence and generating circularized, barcode-tagged fragments;
(e) fragmenting the circularized, barcode-tagged fragments into a second set of linear, barcode-tagged fragments;
(f) attaching a second adapter to each end of each of the second set of linear, barcode-tagged fragments to form double adapter-ligated barcode-tagged nucleic acid fragments, each double adaptor-ligated barcode-tagged nucleic acid fragment comprising a plurality of library molecules (100) comprising: (i) a surface pinning primer binding site (120), (ii) a left sample index sequence (160), (iii) a forward sequencing primer binding site (140), (iv) a left unique molecular index (UMI) sequence (180), (v) an insert sequence (110), (vi) a reverse sequencing primer binding site (150), (vii) a right sample index sequence (170), and (viii) a surface capture primer binding site (130);
(g) replicating the double adapter-ligated barcode-tagged nucleic acid fragments;
(h) sequencing the double adapter-ligated barcode-tagged nucleic acid fragments;
(i) sorting a series of sequenced nucleic acid fragments into independent groups of reads; and
(j) assembling each independent group of reads into the longer nucleic acid sequence, thereby obtaining the nucleic acid sequence information.

In some embodiments, the method further comprises generating single stranded library molecules from the plurality of library molecules (100).

In some embodiments, the right sample index sequence (170) includes a 3-mer random sequence.

In some embodiments, step (g) comprises replicating all of the double adapter-ligated barcode-tagged nucleic acid fragments.

In some embodiments, the method further comprises forming a plurality of library-splint complexes (300) comprising:
i) providing a plurality of single-stranded splint strands (200) wherein individual single-stranded splint strands (200) in the plurality comprise a first region (210) that is capable of hybridizing with the at least a first left universal adaptor sequence (120) of an individual library molecule, and a second region (220) that is capable of hybridizing with the at least a first right universal adaptor sequence (130) of the individual library molecule;
ii) hybridizing the plurality of single-stranded splint strands (200) with plurality of single-stranded nucleic acid library molecules (100) such that the first region of one of the single-stranded splint strands (210) anneals to the at least first left universal adaptor sequence (120) of the library molecule, and such that the second region of the single-stranded splint strand (220) anneals to the at least first right universal sequence (130) of the library molecule, thereby circularizing individual library molecules to form a plurality of library-splint complexes (300) having a nick between the terminal 5′ and 3′ ends of the library molecule, wherein the nick is enzymatically ligatable; and
iii) ligating the nick in the plurality of library-splint complexes (300) thereby generating a plurality of covalently closed circular library molecules (400).

In some embodiments, the method comprises (iv) distributing the plurality of covalently closed circular library molecules (400) onto a support having a plurality of surface primers immobilized on the support, under a condition suitable for hybridizing individual covalently closed circular library molecules (400) to individual immobilized surface primers thereby immobilizing the plurality of covalently closed circular library molecules (400).

In some embodiments, the method further comprises: (v) contacting the plurality of immobilized covalently closed circular library molecules (400) with a plurality of strand-displacing polymerases and a plurality of nucleotides, under a condition suitable to conduct a rolling circle amplification reaction on the support using the plurality of surface primers as immobilized amplification primers and the plurality of covalently closed circular library molecules (400) as template molecules, thereby generating a plurality of immobilized nucleic acid concatemer molecules.

In some embodiments, step (h) comprises sequencing the plurality of immobilized nucleic acid concatemer molecules.

In some embodiments, the sequencing the plurality of immobilized nucleic acid concatemer molecules further comprises:
a) contacting the plurality of immobilized concatemer molecules with (i) a plurality of sequencing polymerases and (ii) a plurality of the soluble sequencing primers, wherein the contacting is conducted under a condition suitable to form a plurality of complexed polymerases each comprising a sequencing polymerase bound to a nucleic acid duplex wherein the nucleic acid duplex comprises a concatemer molecule hybridized to a soluble sequencing primer;
b) contacting the plurality of complexed sequencing polymerases with a plurality of nucleotides under a condition suitable for binding at least one nucleotide to a complexed sequencing polymerase, wherein the plurality of nucleotides comprises at least one nucleotide analog labeled with a fluorophore and having a removable chain terminating moiety at the sugar 3′ position;
c) incorporating at least one nucleotide into the 3′ end of the hybridized sequencing primers thereby generating a plurality of nascent extended sequencing primers; and
d) detecting the incorporated nucleotide and identifying the nucleo-base of the incorporated nucleotide.

In some embodiments, the sequencing the plurality of immobilized nucleic acid concatemer molecules further comprises:
a) contacting the plurality of immobilized concatemer molecules with (i) a plurality of sequencing polymerases and (ii) a plurality of the soluble sequencing primers, wherein the contacting is conducted under a condition suitable to form a plurality of first complexed polymerases each comprising a sequencing polymerase bound to a nucleic acid duplex, wherein the nucleic acid duplex comprises a concatemer molecule hybridized to a soluble sequencing primer;
b) contacting the plurality of complexed sequencing polymerases with a plurality of detectably labeled multivalent molecules to form a plurality of multivalent-complexed polymerases, under a condition suitable for binding complementary nucleotide units of the multivalent molecules to at least two of the plurality of first complexed polymerases thereby forming a plurality of multivalent-complexed polymerases, and the condition inhibits incorporation of the complementary nucleotide units into the sequencing primers of the plurality of multivalent-complexed polymerases, wherein individual multivalent molecules in the plurality of multivalent molecules comprise a core attached to multiple nucleotide arms and each nucleotide arm is attached to a nucleotide unit;
c) detecting the plurality of multivalent-complexed polymerases; and
d) identifying the nucleo-base of the complementary nucleotide units that are bound to the plurality of first complexed polymerases in the plurality of multivalent-complexed polymerases, thereby determining the sequence of the nucleic acid template.

In some embodiments, the method further comprises:
e) dissociating the plurality of multivalent-complexed polymerases and removing the plurality of first sequencing polymerases and their bound multivalent molecules, and retaining the plurality of nucleic acid duplexes;
f) contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a retained nucleic acid duplex;
g) contacting the plurality of second complexed polymerases with a plurality of non-labeled nucleotides, wherein the contacting is conducted under a condition suitable for binding complementary nucleotides from the plurality of nucleotides to at least two of the second complexed polymerases of step (f) thereby forming a plurality of nucleotide-complexed polymerases and the condition is suitable for promoting incorporation of the bound complementary nucleotides into the sequencing primers of the nucleotide-complexed polymerases.

In some embodiments, the method comprises:
a) binding a first universal nucleic acid primer, a first DNA polymerase, and a first multivalent molecule to a first portion of the concatemer molecules, thereby forming a first binding complex, wherein a first nucleotide unit of the first multivalent molecule binds to the first DNA polymerase; and
b) binding a second universal nucleic acid primer, a second DNA polymerase, and the first multivalent molecule to a second portion of the same concatemer template molecule thereby forming a second binding complex, wherein a second nucleotide unit of the first multivalent molecule binds to the second DNA polymerase, wherein the first and second binding complexes which include the same multivalent molecule forms an avidity complex, wherein the first multivalent molecule comprises a core attached to multiple nucleotide arms and each nucleotide arm is attached to a nucleotide unit, and wherein the concatemer molecule comprises two or more tandem repeat sequences of a sequence of interest (110) and a universal primer binding site that binds the first and second universal nucleic acid primers.

In some embodiments, the method comprises:
a) binding a first universal nucleic acid primer, a first DNA polymerase, and a first multivalent molecule to a first portion of the concatemer molecules, thereby forming a first binding complex, wherein a first nucleotide unit of the first multivalent molecule binds to the first DNA polymerase; and
b) binding a second universal nucleic acid primer, a second DNA polymerase, and the first multivalent molecule to a second portion of the same concatemer template molecule thereby forming a second binding complex, wherein a second nucleotide unit of the first multivalent molecule binds to the second DNA polymerase, wherein the first and second binding complexes which include the same multivalent molecule forms an avidity complex, wherein the first multivalent molecule comprises a core attached to multiple nucleotide arms and each nucleotide arm is attached to a nucleotide unit, and wherein the concatemer molecule comprises two or more tandem repeat sequences of a sequence of interest (110) and a universal primer binding site that binds the first and second universal nucleic acid primers, and wherein the contacting is conducted under a condition suitable to inhibit polymerase-catalyzed incorporation of the bound first and second nucleotide units in the first and second binding complexes;
c) detecting the first and second binding complexes on the same concatemer template molecule, and identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

In some embodiments, nucleic acid sequence information is obtained for a longer nucleic acid sequence comprising a length of at least 500 bases. In some embodiments, nucleic acid sequence information is obtained for a longer nucleic acid sequence comprising a length of at least 1,000 bases. In some embodiments, nucleic acid sequence information is obtained for a longer nucleic acid sequence comprising a length from about 1,000 bases to about 40,000 bases. In some embodiments, nucleic acid sequence information is obtained for a longer nucleic acid sequence comprising a length of up to about 35 kilobases. In some embodiments, the nucleic acid sequence information is obtained from about 5,000 to about 25,000 independent groups of reads.

In some embodiments, a longer nucleic acid sequence resulting from the method is about two-fold longer than a nucleic acid sequence resulting from an alternate method for obtaining nucleic acid sequence information. In some embodiments, the method provides about a two-fold increase in the amount of reads in comparison to an alternate method for obtaining nucleic acid sequence information.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings or figures (also “FIG.” and “FIGs.” herein), of which:

FIG. 1A shows a schematic illustration of an example method for assembling sequences of individual nucleic acid molecules.

FIG. 1B shows example sequencing data demonstrating that barcode pairing can improve assembly lengths.

FIG. 1C provides example length histograms of the contiguous sequences (“contigs”) assembled from genomic reads (minimum lengths of about 1000 bps) from E. coli MG1655 (top panel) and Gelsemium sempervirens (bottom panel).

FIG. 2 shows an example three-dimensional scatter plot (inset) showing barcode fidelity in sequencing results from a mixture of three homologous 3-kb plasmids (i.e., three target nucleic acid molecules).

FIG. 3 is a detailed schematic of an example conversion of sheared circular DNA into a sequencing-ready library.

FIG. 4 is a schematic diagram showing example linear amplification of nucleic acid sequence prior to exponential PCR to reduce amplification bias.

FIG. 5 is a schematic diagram showing an example approach used to attach the same barcode to both ends of a target molecule.

FIG. 6 is a schematic diagram showing another example approach used to attach the same barcode to both ends of a target molecule, by creating a circularizing barcode adapter containing two full copies of the same degenerate barcode.

FIG. 7 is a schematic diagram showing an example approach for incorporating barcodes into full-length cDNA during reverse-transcription.

FIG. 8A is a schematic diagram of an example method for fragment generation based on extension of random primers.

FIG. 8B continues from FIG. 8A and completes the example method of fragment generation based on extension of random primers.

FIG. 9 schematically depicts an example computer control system described herein.

FIG. 10 is a schematic showing an exemplary linear single stranded library molecule (100) hybridizing with a single-stranded splint molecule/strand (200) thereby circularizing the library molecule to form a library-splint complex (300) with a nick. The library molecule (100) comprises: (i) a surface pinning primer binding site (120), (ii) a left sample index sequence (160), (iii) a forward sequencing primer binding site (140), (iv) a left unique molecular identifier (UMI) sequence (180), (v) an insert sequence (e.g., sequence of interest) (110), (vi) a reverse sequencing primer binding site (150), (vii) a right sample index sequence (170) which optionally includes a 3-mer random sequence, and (viii) a surface capture primer binding site (130). The single-stranded splint strand (200) comprises: (i) a first region (210) having a universal binding sequence that hybridizes with a sequence on one end of the linear single stranded library molecule, for example the surface pinning primer binding site (120); and (ii) a second region (220) having a universal binding sequence that hybridizes with a sequence on the other end of the linear single stranded library molecule, for example the surface capture primer binding site (130).

FIG. 11 is a schematic showing an exemplary single-stranded splint strand (200) comprising a first region (210) carrying the sequence 5′-ACCCTGAAAGTACGTGCATTACATG-3′ (SEQ ID NO:25), and a second region (220) carrying the sequence 5′-GATCAGGTGAGGCTGCGACGACT-3′ (SEQ ID NO:26).

FIG. 12 is a schematic showing an exemplary library-splint complex (300) undergoing a ligation reaction to close the nick to form a covalently closed circular library molecule (400) which is hybridized to a single-stranded splint strand (200), where the single-stranded splint strand (200) is used as an amplification primer to conduct a rolling circle amplification reaction. The dotted line represents the nascent extension product.

FIG. 13 is a schematic showing an exemplary linear single stranded library molecule (500) hybridizing with a double-stranded splint adaptor (600) thereby circularizing the library molecule to form a library-splint complex (900) with two nicks. The library molecule (500) comprises: (i) a surface pinning primer binding site (520), (ii) a left sample index sequence (560), (iii) a forward sequencing primer binding site (540), (iv) a left UMI sequence (580), (v) an insert sequence (e.g., sequence of interest) (510), (vi) a reverse sequencing primer binding site (550), (vii) a right sample index sequence (570) which optionally includes a 3-mer random sequence, and (viii) a surface capture primer binding site (530). The double-stranded splint adaptors (600) comprise a first splint strand (e.g., a long splint strand) (700) hybridized to a second splint strand (e.g., a short splint strand) (800).

FIG. 14 is a schematic showing an exemplary double-stranded splint adaptor (600) comprising a first splint strand (700) having a sequence 5′-TCGGTGGTCGCCGTATCATTACCCTGAAAGTACGTGCATTACATGGATCAGGTGAGG CTGCGACGACTCAAGCAGAAGACGGCATACGA-3′ (SEQ ID NO:42), and a second splint strand (800) having a sequence 5′-AGTCGTCGCAGCCTCACCTGATCCATGTAATGCACGTACTTTCAGGGT-3′ (SEQ ID NO:45).

FIG. 15A-15C is a schematic showing an exemplary library-splint complex (900) undergoing a ligation reaction to close the two nicks to form a covalently closed circular library molecule (1000) which is hybridized to a first splint strand (700), where the first splint strand (700) is used as an amplification primer to conduct a rolling circle amplification reaction. The dotted line represents the nascent extension product.

FIG. 16 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated ‘B’, and streptavidin is designated ‘SA’.

FIG. 17 is a schematic of an exemplary multivalent molecule comprising a generic core attached to a plurality of nucleotide-arms.

FIG. 18 is a schematic of an exemplary multivalent molecule comprising a dendrimer core attached to a plurality of nucleotide-arms.

FIG. 19 shows a schematic of an exemplary multivalent molecule comprising a core attached to a plurality of nucleotide-arms, where the nucleotide arms comprise biotin, spacer, linker, and a nucleotide unit.

FIG. 20 is a schematic of an exemplary nucleotide-arm comprising a core attachment moiety, spacer, linker, and nucleotide unit.

FIG. 21 shows the chemical structure of an exemplary spacer (top), and the chemical structures of various exemplary linkers, including an 11-atom Linker, a 16-atom Linker, a 23-atom Linker, and an N3 Linker (bottom).

FIG. 22 shows the chemical structures of various exemplary linkers including Linkers 1-9.

FIG. 23 shows the chemical structures of various exemplary linkers joined or attached to nucleotide units.

FIG. 24 shows the chemical structures of various exemplary linkers joined or attached to nucleotide units.

FIG. 25 shows the chemical structures of various exemplary linkers joined or attached to nucleotide units.

FIG. 26 shows the chemical structures of various exemplary linkers joined or attached to nucleotide units.

FIG. 27 shows the chemical structure of an exemplary biotinylated nucleotide-arm. In this example, the nucleotide unit is connected to the linker via a propargyl amine attachment at the 5 position of a pyrimidine base or the 7 position of a purine base.

FIG. 28 is a schematic of an exemplary low binding support comprising a substrate and alternating layers of hydrophilic coatings which are adhered (e.g., covalently or non-covalently) to the glass, and which further comprises chemically reactive functional groups that serve as attachment sites for oligonucleotide primers (e.g., capture oligonucleotides).

FIG. 29 is a schematic of a guanine tetrad (e.g., G-tetrad).

FIG. 30 is a schematic of an exemplary intramolecular G-quadruplex structure.

FIG. 31A is a contig length histogram showing all UMI-tagged contigs from a Rhodobacter sphaeroides sample and sequenced on an Illumina NextSeq™ 550 sequencing apparatus using a sequencing method that employed fluorophore-labeled chain terminating nucleotides.

FIG. 31B is a contig length histogram showing all UMI-tagged contigs from a Rhodobacter sphaeroides sample and sequenced on an AVITI™ sequencing apparatus (from Element Biosciences™) using a two-stage sequencing method.

FIG. 32A is a contig length histogram showing all UMI-tagged contigs from an environmental gDNA sample and sequenced on an Illumina NextSeq™ 550 sequencing apparatus using a sequencing method that employed fluorophore-labeled chain terminating nucleotides.

FIG. 32B is a contig length histogram showing all UMI-tagged contigs from an environmental gDNA sample and sequenced on an AVITI™ sequencing apparatus (from Element Biosciences™) using a two-stage sequencing method.

FIG. 33A is a contig length histogram showing all UMI-tagged contigs from an environmental gDNA sample and sequenced on an Illumina NextSeq™ 550 sequencing apparatus using a sequencing method that employed fluorophore-labeled chain terminating nucleotides.

FIG. 33B is a contig length histogram showing all UMI-tagged contigs from an environmental gDNA sample and sequenced on an AVITI™ sequencing apparatus (from Element Biosciences™) using a two-stage sequencing method.

FIG. 34A is a contig length histogram showing all UMI-tagged contigs from a sample encoding an antibody and sequenced on an Illumina NextSeq™ 550 sequencing apparatus using a sequencing method that employed fluorophore-labeled chain terminating nucleotides.

FIG. 34B is a contig length histogram showing all UMI-tagged contigs from a sample encoding an antibody and sequenced on an AVITI™ sequencing apparatus (from Element Biosciences™) using a two-stage sequencing method.

FIG. 35A is a contig length histogram showing all UMI-tagged contigs from a sample encoding an antibody and sequenced on an Illumina NextSeq™ 550 sequencing apparatus using a sequencing method that employed fluorophore-labeled chain terminating nucleotides.

FIG. 35B is a contig length histogram showing all UMI-tagged contigs from a sample encoding an antibody and sequenced on an AVITI™ sequencing apparatus (from Element Biosciences™) using a two-stage sequencing method.

FIG. 36A is a contig length histogram showing all UMI-tagged contigs from a sample encoding an antibody and sequenced on an Illumina NextSeq™ 550 sequencing apparatus using a sequencing method that employed fluorophore-labeled chain terminating nucleotides.

FIG. 36B is a contig length histogram showing all of the UMI-tagged contigs from a sample encoding an antibody and sequenced on an AVITI™ sequencing apparatus (from Element Biosciences™) using a two-stage sequencing method.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Aspects of the disclosure described with “a” or “an” should be understood to include “one or more” unless the context clearly requires a narrower meaning.

The disclosure provides an improved method for obtaining nucleic acid sequence information. In various aspects, the method permits the quicker and more accurate assembly of intermediate and long read lengths of target nucleic acids from short nucleic acid sequences.

The disclosure also provides methods for obtaining nucleic acid sequence information by reconstructing intermediate and/or long nucleic acid sequences from the assembly of short or intermediate nucleic acid sequences.

The sequencing methods of the present disclosure provide numerous technical advantages over sequencing methods of the prior art. Surprisingly, such advantages are most demonstrable in high-complexity sequencing scenarios. For example, sequencing a bacterial genome using a method of the disclosure (e.g., Element Biosystems™ AVITI™) provides about twice as many reads as a sequencing method of the prior art (e.g., Illumina™ NextSeq™ 550). In another example, sequencing of environmental gDNA (e.g., a heterogenous population of bacteria), using a method of the disclosure (e.g., Element Biosystems™ AVITI™) provides about twice as many reads as a sequencing method of the prior art (e.g., Illumina™ NextSeq™ 550). A sequencing method of the disclosure (e.g., Element Biosystems™ AVITI™) also provides contigs that are about 2-fold in length as compared to a contigs resulting from a method of the prior art (e.g., Illumina™ NextSeq™ 550). A “contig” and “longer nucleic acid sequence” may be used interchangeably in the present disclosure.

In some embodiments, sequencing methods of the present disclosure (e.g., Element Biosystems™ AVITI™) provide about twice as many reads (e.g., about 2-fold, about 2.25-fold, about 2.5-fold, or more than 3-fold) as a sequencing method of the prior art (e.g., Illumina™ NextSeq™ 550). In some embodiments, sequence information is obtained from at least 5,000 reads, at least 7,500 reads, at least 10,000 reads, at least 15,000 reads, at least 20,000 reads, at least 25,000 reads, or any range of reads therebetween.

In some embodiments, a contig of a method of the disclosure (e.g., Element Biosystems™ AVITI™) is about 2-fold, about 2.25-fold, about 2.5-fold, or more than 3-fold that of a contig resulting from a method of the prior art (e.g., Illumina™ NextSeq™ 550). In some embodiments, a contig of the present disclosure is at least 500 bases, e.g., at least 500 bases, at least 600 bases, at least 700 bases, at least 800 bases, at least 900 bases, at least 1,000 bases, at least 1,500 bases, at least 2,000 bases, at least 2,500 bases, at least 5,000 bases, at least 7,500 bases, at least 10,000 bases, at least 15,000 bases, at least 20,000 bases, at least 25,000 bases, at least 30,000 bases, at least 35,000 bases, 40,000 or more bases, or any range of bases therebetween. A “contig” and “longer nucleic acid sequence” may be used interchangeably in the present disclosure.

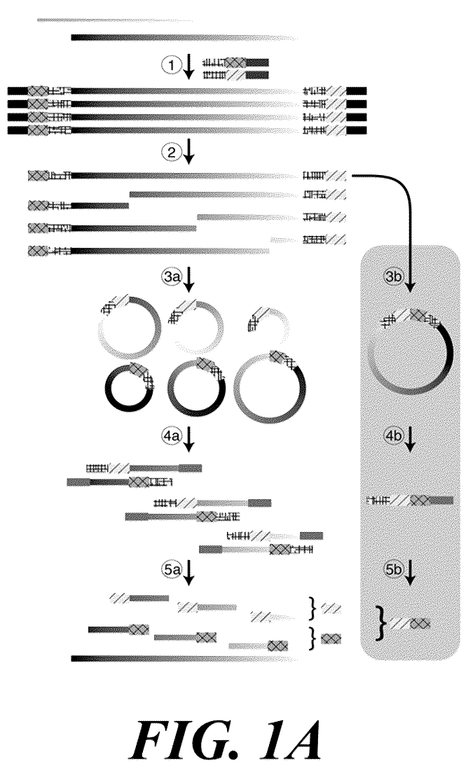

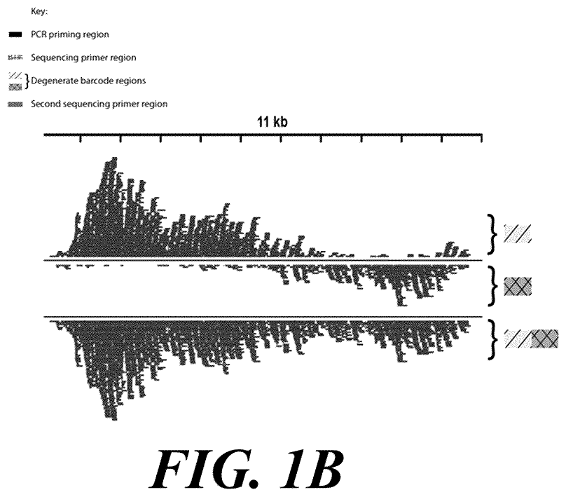

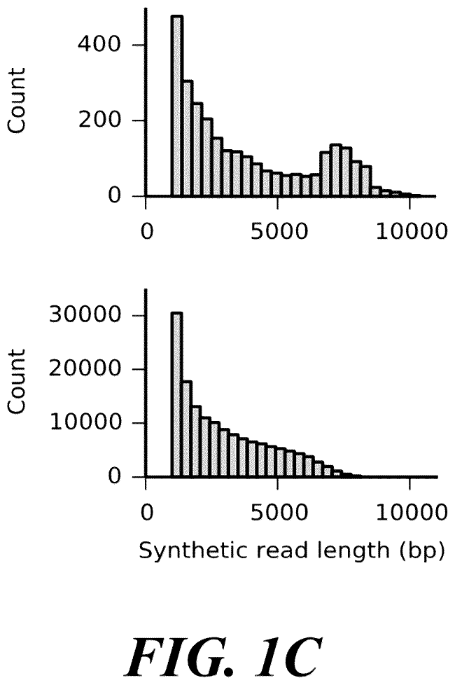

FIG. 1A and FIG. 1B provide an illustration of an example embodiment of the disclosure and shows how barcode pairing (as described herein) improves sequence assembly of long nucleic acid sequences. FIG. 1A shows a schematic illustration of a method for assembling sequences of individual nucleic acid molecules. Mixed target molecules are tagged with tripartite adapters comprising an outer PCR priming region (black bar), an inner region containing a sequencing primer region (shaded bars), and a central degenerate barcode region (diagonal bars and diamond bars). PCR is carried out generating many copies of each tagged molecule (1 in FIG. 1A). The priming region is removed by enzymatic digestion and a single break (on average) is made in each copy of the tagged molecule (2 in FIG. 1A). Tagged nucleic acid molecules are circularized (3a in FIG. 1A) bringing the newly exposed end of the fragment into proximity with the barcode. Circularized, tagged nucleic acid molecules are linearized; a second sequencing primer/adapter (grey bar) is added; and sequencing-ready libraries are prepared (4a in FIG. 1A). Sequence reads begin with the barcode sequence and continue into the unknown region. Short reads are grouped by common barcodes to assemble the original target molecule (5a in FIG. 1A). A barcode-pairing protocol (grey box) is used to resolve the two distinct barcodes affixed to each original target molecule. Circularization of unbroken copies (3b in FIG. 1A) brings the two barcodes together. Subsequent sequencing reads contain both barcode sequences (4b in FIG. 1A), allowing the two barcode-defined groups to be collapsed into a single group (5b in FIG. 1A).

FIG. 1B shows that barcode pairing can improve assembly lengths. Reads associated with two distinct barcodes are shown aligned to the MG1655 reference genome. Individually, each group of reads (top) assembles into a contiguous sequence (“contig”) about 6 kb in length. Barcode pairing merges the groups (bottom), increasing and smoothing coverage across the region to allow assembly of the full 10-kb target sequence. FIG. 1C provides length histograms of the contigs assembled from genomic reads (minimum length of about 1000 bp) from E. coli MG1655 (top panel) and Gelsemium sempervirens (bottom panel). The N50 length of the synthetic reads for E. coli MG1655 is 6.0 kb, and the longest synthetic read (contig) in this example is 11.6 kb. The N50 length of the synthetic reads is 4.0 kb. In some embodiments, the number of possible barcode sequences is 4ⁿ, where n is the number of degenerate bases. It is contemplated that the number (n) should be at least 100 times higher than the number of DNA molecules to be tagged to ensure that each molecule receives two unique tags. For example, n=16 has been used in certain experiments described herein (4¹⁶=4.3 billion). In various aspects, the barcode is made shorter (to maximize the portion of the sequencing read that reads target sequence) or longer (to ensure that no two molecules get identical barcodes).

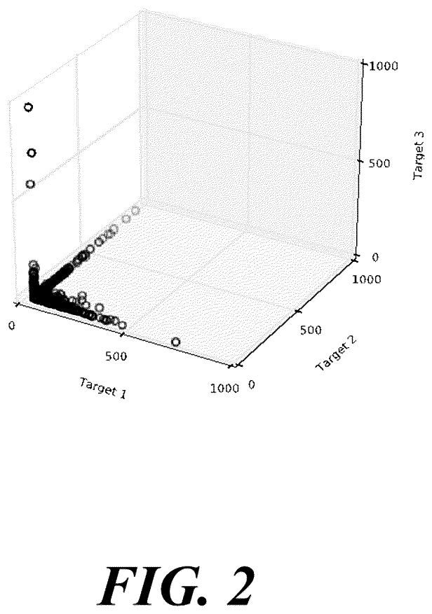

FIG. 2 shows an example three-dimensional scatter plot (inset) showing barcode fidelity in sequencing results from a mixture of three homologous 3-kb plasmids (i.e., three target nucleic acid molecules). The reads associated with each barcode were searched for short sequences unique to each variant. Each point represents a different barcode (about 8,000 total), and its position indicates the number of times sequences unique to each of three mixed target molecules were found within that set of barcode-grouped reads. Counting the barcodes associated with each target molecule provides a measurement of mixture composition. For example, although Target 3 was rare in the mixture, the barcodes that tagged Target 3 had as many counts as barcodes tagging more abundant targets.

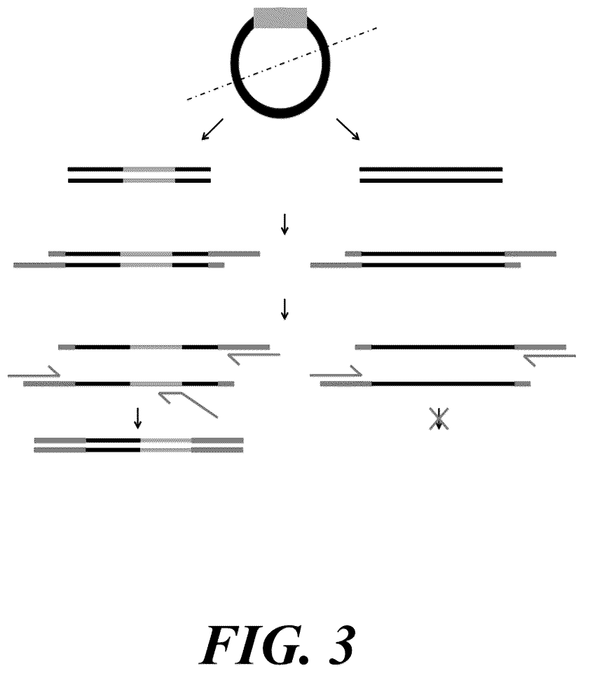

FIG. 3 is a detailed schematic of an aspect of the disclosure showing example conversion of sheared circular DNA into a sequencing-ready library. Circularized DNA (black) containing barcode and annealing sequences (grey) is fragmented (dotted line) into molecules of about 500 bp in length. Some of the resulting molecules will contain a barcode and others will not. Asymmetric adapters are ligated to each end of the molecules. Limited-cycle PCR is performed with a first primer complementary to the asymmetric adapter and a second primer complementary to the internal annealing sequence from the tripartite adapter. The primers add the full sequencing adapter sequences to the PCR product. Only molecules containing internal annealing sequences and barcodes are exponentially amplified in the PCR.

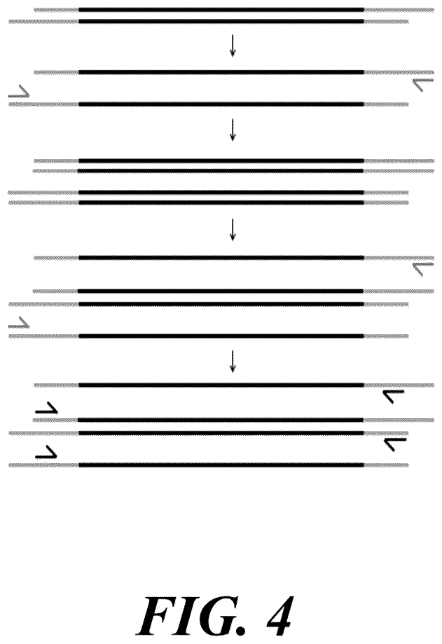

FIG. 4 is a schematic diagram of an aspect of the disclosure showing example linear amplification of nucleic acid sequence prior to exponential PCR to reduce amplification bias. In some aspects, the tripartite adapter is designed with an overhang containing an annealing region for a linear amplification primer (grey arrows). Each round of thermocycling in the presence of this primer copies the original adapter ligated molecules. However, the newly synthesized copies will not themselves be copied because they do not have the annealing site for the linear amplification primer. Exponential PCR can be triggered by the addition of a second primer (black arrows).

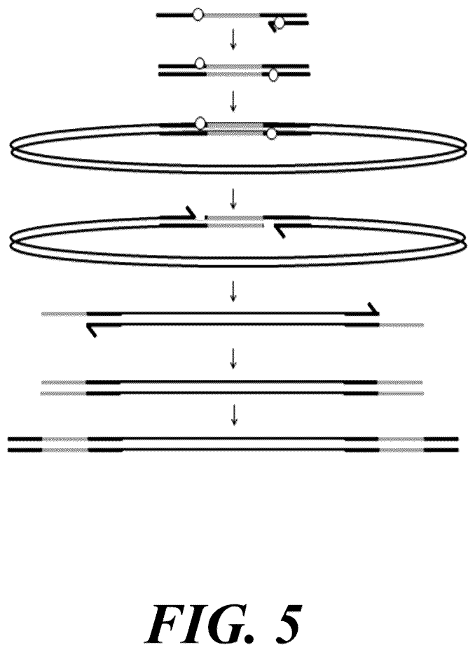

FIG. 5 is a schematic diagram of an aspect of the disclosure showing an example approach used to attach the same barcode to both ends of a target molecule. An oligonucleotide is synthesized containing a uracil base (white circle) and a degenerate barcode region (grey region). A second oligonucleotide is synthesized to contain a uracil base and to be complementary to a region of the first oligonucleotide. The second oligonucleotide anneals to the first and is extended by a DNA polymerase, copying the barcode region and forming a double-stranded molecule. The target molecule is circularized around the double-stranded adapter. An enzyme, for example, USER™ enzyme, excises the uracil bases, creating nicks in each strand, and opening the circular molecule into a linear molecule. DNA polymerase extends the new 3′ ends, copying the single-stranded barcode regions to create a fully double-stranded molecule. An additional adapter containing a PCR primer annealing sequence is ligated onto both ends of the linear molecule. The end result is a linear molecule comprising the same barcode on both ends.

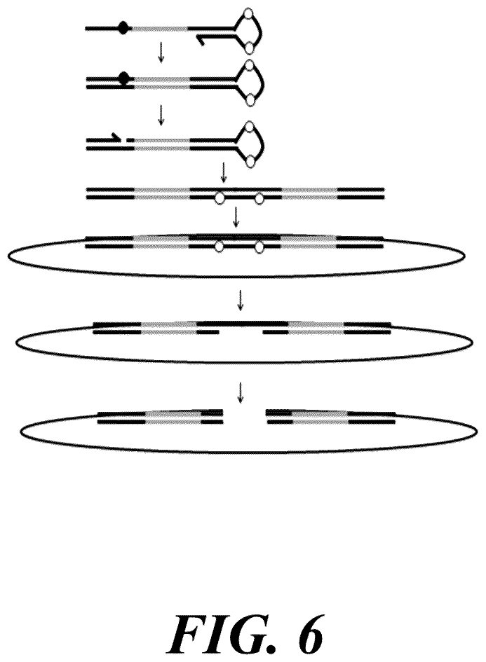

FIG. 6 is a schematic diagram of an aspect of the disclosure showing another example approach used to attach the same barcode to both ends of a target molecule, by creating a circularizing barcode adapter containing two full copies of the same degenerate barcode. An oligonucleotide (i.e., “oligo”) is synthesized to contain a nicking endonuclease site (black circle), a degenerate barcode (grey), a self-priming hairpin, and two or more uracil bases (white circles). The self-priming 3′ end is extended with DNA polymerase, copying the barcode sequence. The DNA is nicked at the newly double-stranded nicking endonuclease site, creating a free 3′ end. The free 3′ end is extended by a strand-displacing DNA polymerase, which copies the barcode sequence yet again. The target molecule is circularized around the barcode adapter by ligation. In some aspects, a USER™ enzyme excises two or more uracil bases from the original synthetic strand, creating a single-stranded gap. S1 nuclease or mung bean nuclease degrades the single-stranded DNA, opening the circle into a linear molecule comprising identical barcodes at both ends.

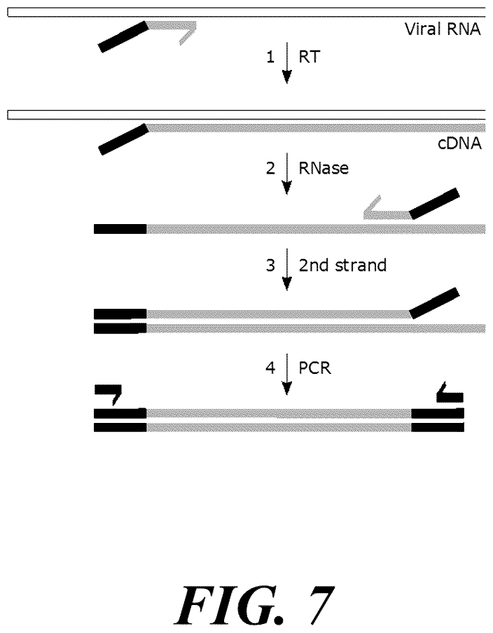

FIG. 7 is a schematic diagram of an aspect of the disclosure showing an example approach for incorporating barcodes to full-length cDNA during reverse-transcription. (1) RNA (white) is reverse transcribed (RT) from a primer comprising an annealing portion (grey) and a tripartite overhang portion (black) containing a barcode. (2) Following 1st strand synthesis, the RNA is degraded by RNase treatment and excess primers are removed. (3) A second tripartite barcode-containing primer is added and the second strand is synthesized. (4) Excess, unbound primers are removed, and full-length cDNA is exponentially amplified by PCR with a third primer (black arrows) complimentary to adapters on both strands.

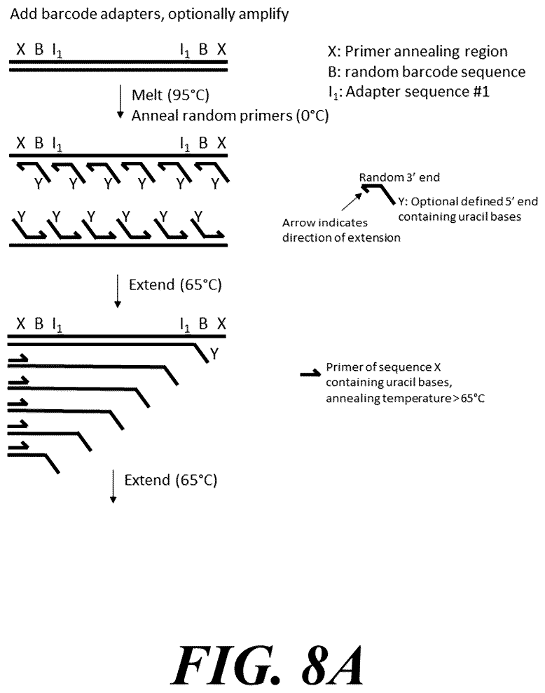

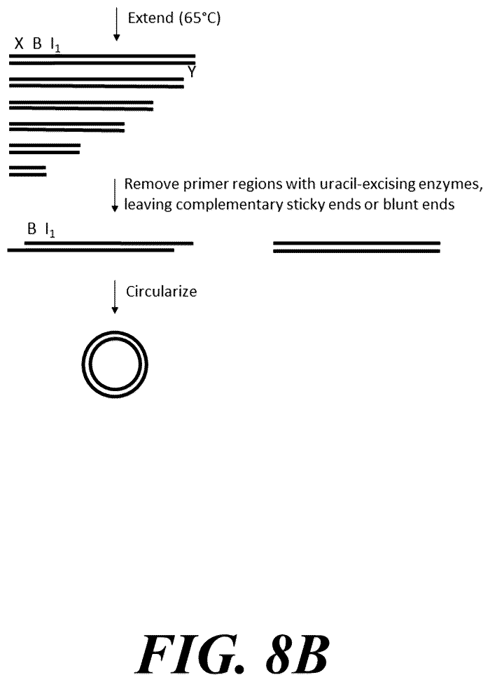

FIG. 8A and FIG. 8B schematically depict an alternate, example approach to creating fragments that relies on extension of random primers rather than breaking full-length copies. Following adapter attachment and optionally PCR, the strands are denatured, and random primers are annealed along the length of the target molecule. The primers can be designed with a random sequence at the 3′ end (e.g., N4 to N₈) and optionally a defined sequence at the 5′ end that is the reverse complement of the sequence at the ends of the target molecule (denoted by “X” in the figure) and contains uracil bases. Extension of the random primers with a strand-displacing polymerase creates single-stranded fragments with one random end defined by the annealing site of the random primer and a second end defined by the termination of extension at end of the target fragment. Second-strand synthesis with an additional primer with a sequence corresponding to X and containing one or more uracil bases can create double-stranded fragments. Both extension rounds can be performed at a relatively high temperature to prevent further annealing of the random primers. The double-stranded fragments can be circularized by blunt-end ligation, or if the X-complementary overhangs were used, USER™ enzyme mix (New England Biolabs™) can be used to excise the uracil-containing regions to produce sticky ends to increase circularization efficiency.

In some embodiments, with random primers, randomly determined ends are created by annealing primers of random or partially random sequences. Each such primer anneals to a complimentary region of the target molecule and is extended by a polymerase. In some cases, the polymerase is capable of strand displacement. In some instances, Bst polymerase is used. In some embodiments, phi29 polymerase is used. In some cases, Vent polymerase is used. In some embodiments, this operation is preceded by linear or exponential amplification of the targets. In some embodiments, the targets are not amplified beforehand. In some cases, a mixture including template molecules and random primers is melted at 95° C. and quenched to 0° C. to allow primer annealing. Bst polymerase can be added and the mixture can be slowly warmed to 65° C. by ramping or stepping. In some cases, primers complementary to the adapter ends of the target are present or are added, and prime the single-stranded DNA synthesized following random priming at its 3′ end. Extension by a DNA polymerase generates double-stranded DNA fragments with the known adapter end sequence at one end and a random sequence from the interior of the target molecule at the other end. In some embodiments, multiple rounds of this linear amplification and fragment generation are performed. In some embodiments, additional rounds are performed by heating the mixture to, e.g., 95° C., to melt the double-stranded DNA duplexes, cooling to promote random primer annealing, and if necessary, adding an additional DNA polymerase. In some embodiments, the target molecule adapters contain one or more biotinylated nucleotides that allow them to specifically bind to streptavidin-coated beads, so that the newly generated fragments can be easily separated from the original targets between rounds of amplification. In some embodiments, the random primers contain defined sequences at their 5′ end and random sequences at their 3′ end, so that the resulting ssDNA or dsDNA contains known sequences at both ends. In some embodiments, the known sequences are the same. In some embodiments, they are different. In some cases, fragments are subsequently amplified by PCR using one or more primers complementary to the known end sequences. In some embodiments, DNA fragments created by linear or exponential amplification contain known end sequences that are reverse complements of each other and contain one or more deoxyuracil bases in the 5′ ends. A combination of uracil-DNA glycosylase (UDG) and exonuclease VIII can then be used to remove the 5′ ends, leaving long single-stranded complimentary sequences that can anneal to increase the efficiency of intramolecular circularization. In some embodiments, treatment with UDG and exonuclease VIII is preceded by treatment with Klenow fragment or a similar enzyme to remove nontemplated deoxyadenosine bases added to the 3′ ends during extension. In some cases, the known end sequences contain sequences that can be recognized by recombinase enzymes that circularize the fragment by recombination. In some embodiments, circularization is by blunt-end ligation.

In some cases, circularized fragments are fragmented by mechanical or enzymatic (e.g., fragmentase, transposons) methods and prepared for sequencing by ligating adapters and performing lcPCR as described herein.

In some embodiments, circularized fragments are amplified by rolling-circle amplification (RCA) or hyperbranching rolling-circle amplification (HRCA). In some cases, RCA or HRCA is primed with random primers or partially random primers. In some embodiments, amplification is primed by one or more primers of defined sequence. In some instances, amplification is performed in the presence of up to 100% dUTP in place of dTTP, to allow the product to be specifically degraded later. In some embodiments, RCA or HCRA is followed by mechanical or enzymatic fragmentation, adapter ligation, and PCR as described herein. In some embodiments, RCA or HRCA is followed directly by PCR or limited-cycle PCR.

In some embodiments, PCR is primed with one primer complementary to the defined sequence at the 5′ end of the partially random primer used for RCA or HRCA, and a second primer complementary to a sequence in the barcode adapter proximal to the barcode sequence. In some embodiments, the PCR primers are complementary to these sequences, but additionally contain 5′ extensions that add further sequences necessary for sequencing. In some cases, RCA or HCRA products containing deoxyuracil are subsequently degraded to enrich for PCR products.

With reference to FIG. 8A, a mixture of target DNA molecules, with barcode adapters attached to the ends according to methods described herein, is prepared with the desired complexity (number of distinct molecules). The barcode adapters contain an end region of defined sequence (X), a degenerate barcode region (B) that is different for every target molecule but defined for a given individual molecule, and a defined region (I₁) complementary to some or all of one of the two eventual sequencing primers, such as a standard sequencing primer (e.g., Illumina™) or a custom primer. Optionally, the molecules are amplified by linear or exponential methods to create 10¹-10⁵ copies (e.g., 10, 10², 10³, 10⁴, or 10⁵ copies) of each uniquely barcoded molecule. The target molecules may be melted into single-stranded DNA, e.g., by heating or exposure to alkaline or other denaturing conditions. One or more random or partially random primers may then be annealed along the length the target molecules by rapid quenching to 0-4° C. The primers depicted here as a non-limiting example are partially random, with a random 3′ region and a defined 5′ region (e.g., sequence Y).

Continuing with FIG. 8A and FIG. 8B, a strand-displacing DNA polymerase, such as Bst DNA polymerase, is added to the primer-annealed target DNA mixture. The temperature is ramped or stepped up to about 65° C., and the polymerase extends each of the random 3′ primer ends annealed along the length of the target molecule, displacing extended molecules in front of it as it goes, releasing them into solution. In some embodiments, one end of the newly synthesized single-stranded DNA molecules is defined by the partially random primer and contains the Y sequence followed by a sequence complementary to the region of the target molecule to which a specific primer from the degenerate mixture annealed. The other end of such embodiments is defined by a sequence complementary to the end sequence of the target molecule, which comprises I₁-B-X. A primer with a sequence complementary to X may be present in the mixture, and when present is designed with an annealing temperature greater than 65° C., allowing it to anneal to the ends of the newly synthesized displaced molecules and prime synthesis of the second strand, creating double-stranded DNA. In certain embodiments, accordingly, the result is a collection of target fragments, with no mechanical or enzymatic shearing needed. If desired, multiple cycles of melting, annealing, and strand-displacement amplification can be performed to increase the yield of DNA. If desired, deoxyadenosine overhangs added by the Bst polymerase in a template-independent fashion can be removed by incubation with, e.g., Klenow DNA polymerase to create blunt-ended dsDNA.

Continuing with FIG. 8A and FIG. 8B, fragments synthesized can be circularized by blunt-end ligation. Alternatively, to improve circularization efficiency of long fragments, sticky-end ligation can be performed. If sequences X and Y in the partially random primers and the second-strand primers are synthesized so that they contain deoxyuracil bases, the USER™ enzyme mix (UDG and endonuclease VIII) can excise the 5′ ends of each strand of the dsDNA to leave sticky ends of programmable length. In some embodiments, if X and Y are reverse complements, the sticky ends will be complementary, and will anneal to one another to promote ligation.

### Nucleic Acids and Nucleic Acid Libraries

A nucleic acid or nucleic acid molecule, as used herein, can include any nucleic acid of interest. In some embodiments, nucleic acids include, but are not limited to, DNA, RNA, peptide nucleic acid, morpholino nucleic acid, locked nucleic acid, glycol nucleic acid, threose nucleic acid, mixtures thereof, and hybrids thereof. In some aspects, a nucleic acid is a “primer” capable of acting as a point of initiation of synthesis along a complementary strand of nucleic acid when conditions are suitable for synthesis of a primer extension product.

In some aspects, the nucleic acid serves as a template for synthesis of a complementary nucleic acid, e.g., by base-complementary incorporation of nucleotide units. For example, in some aspects, a nucleic acid comprises naturally occurring DNA (including genomic DNA), RNA (including mRNA), and/or comprises a synthetic molecule including, but not limited to, complementary DNA (cDNA) and recombinant molecules generated in any manner. In some aspects, the nucleic acid is generated from chemical synthesis, reverse transcription, DNA replication or a combination thereof. In some aspects, the linkage between the subunits is provided by phosphates, phosphonates, phosphoramidates, phosphorothioates, or the like. In some embodiments, the linkage between the subunits is provided by nonphosphate groups, such as, but without limitation, peptide-type linkages, e.g., as utilized in peptide nucleic acids (PNAs). In some aspects, the linking groups are chiral or achiral. In some aspects, the polynucleotides have a three-dimensional structure. In some embodiments, suitable three-dimensional structures encompass single-stranded, double-stranded, and triple helical molecules that are, e.g., DNA, RNA, or hybrid DNA/RNA molecules, and double-stranded with single-stranded regions (for example, stem- and loop-structures).

In some aspects, nucleic acids are obtained from any source. In various aspects, nucleic acid molecules are obtained from a single organism or from populations of nucleic acid molecules obtained from natural sources that include one or more organisms. Sources of nucleic acid molecules include, but are not limited to, organelles, cells, tissues, organs, and organisms. In some aspects, when cells are used as sources of nucleic acid molecules, the cells are derived from any prokaryotic or eukaryotic source. Such cells include, but are not limited to, bacterial cells, fungal cells, plant cells (including vegetable cells), protozoan cells, and animal cells. Such animal cells include, but are not limited to, insect cells, nematode cells, avian cells, fish cells, amphibian cells, reptilian cells, and mammalian cells. In some aspects, the mammalian cells include human cells.

Nucleic acids can be obtained using any suitable method known in the art, including, for example and without limitation, those described by Maniatis et al., Molecular Cloning: A Laboratory Manual, Cold Spring Harbor, N.Y., pp. 280-281 (1982). In another non-limiting example, nucleic acids are obtained as described in U.S. Patent Application Publication No. US 2002/0190663. In some aspects, nucleic acids obtained from biological samples are fragmented to produce suitable fragments for analysis as described in the present disclosure.

In some aspects, a nucleic acid of interest or “target nucleic acid” or “target nucleotide sequence” to be sequenced is fragmented or sheared to a desired length. The terms “fragmenting,” “shearing,” or “breaking” are used interchangeably in various aspects herein to mean cutting or cleaving the nucleic acid into at least two smaller pieces or fragments. In various aspects, a nucleic acid is shortened, or broken into fragments of shorter lengths, in the preparation of a high-quality sequencing library or “target library,” which is important in next-generation sequencing (NGS). In various embodiments, a “target library” or “target nucleic acid library” is created. In some embodiments, the target library comprises fragments of a target nucleic acid of interest. The terms “target nucleic acid” or “target nucleotide” or “target nucleotide sequence” are used herein interchangeably to refer to the nucleic acid or nucleotide to be sequenced.

In various aspects, a nucleic acid is fragmented or shortened by physical, chemical, or enzymatic shearing. In various aspects, physical fragmentation is carried out by acoustic shearing, sonication, or hydrodynamic shear. In many aspects, acoustic shearing and sonication are popular physical methods used to shear DNA. In some aspects, the Covaris® instrument (Covaris®, Woburn, MA) is an acoustic device used for breaking DNA into fragments, e.g., fragments of about 100 bp to about 5,000 bp. In other aspects, the Bioruptor® (Denville, NJ) is a sonication device utilized for shearing chromatin, shearing DNA, and disrupting tissues. In certain embodiments, the Bioruptor® permits small volumes of DNA to be sheared to fragments, e.g., about 150 to about 1 kb in length. In some embodiments, Hydroshear™ (Digilab, Marlborough, MA) utilizes hydrodynamic forces to shear DNA. In some aspects, DNA is sheared by nebulizers (Life Tech™, Grand Island, NY), which atomize liquid using compressed air, and results in shearing DNA into fragments of about 100 bp to about 3,000 bp in seconds. In various aspects, enzymatic fragmentation or shearing is carried out by Fragmentase® (NEB™, Ipswich, MA), KAPA Frag Enzyme (KAPA, Wilmington, MA), DNase I, non-specific nuclease, transposase, another restriction endonuclease, or Nextera tagmentation technology (Illumina™, San Diego, CA). In various aspects, chemical fragmentation is carried out. Chemical fragmentation includes, but is not limited to, exposure to heat and divalent metal cations. Chemical shearing is typically reserved for the breakup of long RNA fragments, and is typically performed through the heat digestion of RNA with a divalent metal cation (e.g., magnesium or zinc). In some aspects, the length of the RNA (e.g., about 115 nucleotides to about 350 nucleotides, e.g., about 110, about 115, about 120, about 125, about 130, about 140, about 150, about 160, about 170, about 180, about 190, about 200, about 220, about 240, about 260, about 280, about 300, about 320, about 340, or about 350 nucleotides) is adjusted, e.g., by increasing or decreasing the time of incubation. In some aspects, the nucleic acid molecule is shortened with an exonuclease.

In various aspects, the size of the nucleic acid fragment is a key factor for library construction and sequencing. In various aspects, a sequencing platform and read length is chosen to be compatible with fragment size. In some aspects, size selection of nucleic acids is performed to remove very short fragments or very long fragments.

In various aspects, fragmentation is carried out in various stages of the methods disclosed herein. For example, in some aspects, there are three fragmentation rounds. For example, in some aspects, if genomic DNA is used as a starting material (rather than mRNA or a PCR product), genomic DNA is fragmented in a first fragmentation round into fragments of about 8 kb to about 10 kb (e.g., about 8 kb, about 8.5 kb, about 9 kb, about 9.5 kb, or about 10 kb). In some embodiments, the fragments of about 8 kb to about 10 kb (e.g., about 8 kb, about 8.5 kb, about 9 kb, about 9.5 kb, or about 10 kb) are tagged and amplified, e.g., by PCR. The amplified copies, in various aspects, are further fragmented in a second fragmentation. In certain embodiments, the second fragmentation breaks the copies one time, e.g., somewhere along their length, into fragments of various lengths. In some embodiments, these fragments of various lengths are then circularized, and the circularized fragments are fragmented again in a third fragmentation, e.g., to fragments of about 300 bases to about 800 bases (e.g., about 300 bases, about 400 bases, about 500 bases, about 600 bases, about 700 bases, or about 800 bases).

In various aspects, the fragment size is about 0.1 kilobase (kb), about 0.15 kb, about 0.2 kb, about 0.25 kb, about 0.3 kb, about 0.35 kb, about 0.4 kb, about 0.45 kb, about 0.5 kb, about 0.55 kb, about 0.6 kb, about 0.65 kb, about 0.7 kb, about 0.75 kb, about 0.8 kb, about 0.85 kb, about 0.9 kb, about 0.95 kb, about 1.0 kb, about 1.5 kb, about 2.0 kb, about 2.5 kb, about 3.0 kb, about 3.5 kb, about 4.0 kb, about 4.5 kb, about 5.0 kb, about 5.5 kb, about 6.0 kb, about 6.5 kb, about 7.0 kb, about 7.5 kb, about 8.0 kb, about 8.5 kb, about 9.0 kb, about 9.5 kb, about 10 kb, about 11 kb, about 12 kb, about 13 kb, about 14 kb, about 15 kb, about 16 kb, about 17 kb, about 18 kb, about 19 kb, about 20 kb, about 30 kb, about 40 kb, about 50 kb, about 60 kb, about 70 kb, about 80 kb, about 90 kb, about 100 kb, about 1,000 kb, or longer.

In various aspects, a size selection is carried out. In some aspects, a size-selection is used after shearing genomic DNA into large fragments, to separate nucleic acid fragments of a size of about 8 kb to about 10 kb (e.g., about 8 kb, about 8.5 kb, about 9 kb, about 9.5 kb, or about 10 kb) from smaller fragments; such smaller fragments which would preferentially amplify during PCR and ultimately yield synthetic reads of limited usefulness. In some aspects, a size selection is used after the fragmentation of PCR products, e.g., to enrich the library for fragments of a particular size. In certain embodiments, the size selection and enrichment compensates for diminished circularization efficiency of fragments depending on size. In some aspects, circularization efficiency is reduced if fragment length is too long, e.g., if the fragment is a long nucleotide sequence.

In some aspects, a size selection is carried out using length-dependent binding to solid phase reversible immobilization (SPRI®, Beckman Coulter) beads. In other aspects, size selection is carried out using agarose or polyacrylamide electrophoresis gel purification and isolation. In some embodiments, size selection via gel electrophoresis purification and isolation may be performed manually. In some embodiments, size selection via gel electrophoresis purification and isolation may be performed with an automated system such as BluePippin™ (Sage Science, Beverly, MA) or E-gels (Thermo Fisher Scientific).

As used herein, a “nucleotide unit” or “nucleotide moiety” refers to nucleotides (e.g., dATP, dTTP, dGTP, dCTP, or dUTP), or analogs thereof, comprising comprises a base, sugar and at least one phosphate group. Nucleotide units can be attached to the multivalent molecules used in the sequencing reactions described herein. In general, all nucleotide units attached to the same multivalent molecule will have the same identity (e.g., all A, all T, all C, or all G), although the skilled artisan will appreciate that there may be situations in which a multivalent molecule comprising nucleotide units of differing identity will be advantageous.

The term “long nucleotide sequence,” “long nucleic acid sequence,” or “long read” as used herein refers to any nucleic acid sequence equal to or greater than 20,000 bases (or 20,000 nucleotides, or 20 kilobases, or 20 kb). In some aspects, the long nucleotide sequence is between approximately 20000 bases to approximately 500,000 bases. In some aspects, the long nucleotide sequence is between approximately 25,000 bases to approximately 100,000 bases. In some aspects, the long nucleotide sequence is about 20,000 bases, about 25,000 bases, about 30,000 bases, about 35,000 bases, about 40,000 bases, about 45,000 bases, about 50,000 bases, about 55,000 bases, about 60,000 bases, about 65,000 bases, about 70,000 bases, about 75,000 bases, about 80,000 bases, about 85,000 bases, about 90,000 bases, about 95,000 bases, about 100,000 bases, about 150,000 bases, about 200,000 bases, about 250,000 bases, about 300,000 bases, about 350,000 bases, about 400,000 bases, about 450,000 bases, or about 500,000 bases.

The term “intermediate nucleotide sequence,” “intermediate nucleic acid sequence,” or “intermediate read” as used herein refers to any nucleic acid sequence greater than 1000 bases and less than 20,000 bases. In some aspects, the intermediate nucleotide sequence is between approximately 1,500 bases and approximately 15,000 bases. In some aspects, the intermediate nucleotide sequence is between approximately 2,000 bases to approximately 12,000 bases. In some aspects, the intermediate nucleotide sequence is between approximately 3,000 bases to approximately 11,000 bases. In some aspects, the intermediate nucleotide sequence is between approximately 4,000 bases to approximately 10000 bases. In some aspects, the intermediate nucleotide sequence is about 1050 bases, about 1100 bases, about 1,150 bases, about 1,200 bases, about 1,250 bases, about 1,300 bases, about 1,350 bases, about 1,400 bases, about 1,450 bases, about 1,500 bases, about 1,550 bases, about 1,600 bases, about 1,650 bases, about 1,700 bases, about 1,750 bases, about 1,800 bases, about 1,850 bases, about 1,900 bases, about 1,950 bases, about 2,000 bases, about 2,100 bases, about 2,200 bases, about 2,300 bases, about 2,400 bases, about 2,500 bases, about 3,000 bases, about 3,500 bases, about 4,000 bases, about 4,500 bases, about 5,000 bases, about 5,500 bases, about 6,000 bases, about 6,500 bases, about 7,000 bases, about 7,500 bases, about 8,000 bases, about 8,500 bases, about 9,000 bases, about 9,500 bases, about 10,000 bases, about 11,000 bases, about 12,000 bases, about 13,000 bases, about 14,000 bases, about 15,000 bases, about 16,000 bases, about 17,000 bases, about 18,000 bases, about 19,000 bases, or less than about 20,000 bases.

The term “short nucleotide sequence,” “short nucleic acid sequence,” or “short read” as used herein refers to any nucleic acid sequence less than or equal to 1000 bases or 1000 nucleotides. In some aspects, the short nucleotide sequence is between approximately 25 bases to approximately 1000 bases. In some aspects, the short nucleotide sequence is between approximately 50 bases to approximately 750 bases. In some aspects, the short nucleotide sequence is between approximately 75 bases to approximately 500 bases. In some aspects, the short nucleotide sequence is about 25 bases, about 50 bases, about 75 bases, about 100 bases, about 125 bases, about 150 bases, about 175 bases, about 200 bases, about 250 bases, about 275 bases, about 300 bases, about 325 bases, about 350 bases, about 375 bases, about 400 bases, about 425 bases, about 450 bases, about 475 bases, about 500 bases, about 525 bases, about 550 bases, about 575 bases, about 600 bases, about 675 bases, about 700 bases, about 725 bases, about 750 bases, about 775 bases, about 800 bases, about 825 bases, about 850 bases, about 875 bases, about 900 bases, about 925 bases, about 950 bases, about 975 bases, or about 1,000 bases.

### Adapters and Adapter Attachment

An “adapter” as used herein refers to a relatively short, nucleic acid molecule which is attached to a nucleic acid molecule in various aspects of the disclosure. In some aspects, an adapter comprises a variety of sequence elements including, but not limited to, an amplification primer annealing sequence or complement thereof, a sequencing primer annealing sequence or complements thereof, a barcode sequence, a common sequence shared among multiple different adapters or subsets of different adapters, a restriction enzyme recognition sites, an overhang complementary to a target polynucleotide overhang, a probe binding site (e.g., for attachment to a sequencing platform), a random or near-random sequence (e.g., a nucleotide selected at random from a set of two or more different nucleotides at one or more positions, with each of the different nucleotides selected at one or more positions represented in a pool of adapters comprising the random sequence), and combinations thereof. In some aspects, two or more sequence elements are non-adjacent to one another (e.g., separated by one or more nucleotides), adjacent to one another, partially overlapping, or completely overlapping. In some aspects, adapters contain overhangs designed to be complementary to a corresponding overhang on the molecule to which ligation is desired. In some aspects, a complementary overhang is one or more nucleotides in length including, but not limited to, 1, 2, 3, 4, 5, 6, 7, 8, 9, 10, 11, 12, 13, 14, 15, or more nucleotides in length. In some aspects, a complementary overhang comprises a fixed or a random sequence.

In some aspects, the adapter is a “tripartite adapter” comprising a polymerase chain reaction (PCR) primer region, a sequencing primer region, and a barcode region. In some aspects, the tripartite adapter comprises an outer PCR primer region (or amplification primer region or sequence), an inner sequencing primer region (or sequence), and a central barcode region (or sequence). It is contemplated herein that the use of barcodes improves the levels of sequencing information retained following the shearing of a target nucleic acid into sequencing-compatible fragments. In some aspects, each barcode is specific to the individual intermediate-length nucleic acid molecule from which a given short sequenced nucleic acid molecule is derived and is used to identify the source of the short nucleic acid. In various aspects, therefore, a given barcode is exclusively associated with a single target molecule. Thus, the term “barcode fidelity” as used herein refers to a particular barcode being exclusively associated with a single target molecule. Accordingly, with perfect barcode fidelity, every read tagged with that barcode is derived from that single target molecule and contains nucleotide sequence information from that single target molecule alone. In some embodiments, therefore, when being assembled (e.g., in a computational pipeline), reads sharing a barcode sequence are distinguished from the background of reads without that particular barcode, and are then grouped together and assembled to recreate the sequence of the original longer molecule. As used herein, a “computational pipeline” or “processing pipeline” is a system for processing sequencing data and assembling the short nucleic acid sequence data into synthetic long nucleic acids.

In some aspects, short defined sequences (referred to herein as “constant sequences”) are designed to follow and/or precede the barcode sequence in the sequencing reads to positively distinguish true barcode sequences from spurious sequences. In some aspects, these constant sequences are selected to promote incorporation of biotinylated deoxyribonucleotides (e.g., biotin-dCTP) into the fragmented molecules during end-repair.

In some aspects, an amplification primer annealing sequence also serves as a sequencing primer annealing sequence. In some aspects, sequence elements are located at or near the ligating end, at or near the non-ligating end, or in the interior of the adapter. In some aspects, when an adapter oligonucleotide is capable of forming secondary structure, such as a hairpin, sequence elements are located partially or completely outside the secondary structure, partially or completely inside the secondary structure, or in between sequences participating in the secondary structure. For example, in some aspects, when an adapter oligonucleotide comprises a hairpin structure, sequence elements are located partially or completely inside or outside the hybridizable sequences (the “stem”), including in the sequence between the hybridizable sequences (the “loop”).

In some aspects, the first adapter oligonucleotides in a plurality of first adapter oligonucleotides having different barcode sequences each comprise a sequence element common among all first adapter oligonucleotides in the plurality. In some aspects, all second adapter oligonucleotides comprise a sequence element common among all second adapter oligonucleotides that is different from the common sequence element shared by the first adapter oligonucleotides. In some aspects, a difference in sequence elements comprises any such difference, wherein at least a portion of the different adapters do not completely align. For example and without limitation, the different adapters may not completely align due to changes in sequence length, deletion, or insertion of one or more nucleotides, or a change in the nucleotide composition at one or more nucleotide positions (such as a base change or a base modification).

In some aspects, partial sequencing primer sequences (e.g., sequencing primers like those commercially available from Illumina™ or Element Biosciences) are included adjacent to the random barcode sequence in the barcode adapter. In some aspects, the partial sequence anneals in downstream PCR to a longer oligonucleotide that adds a full sequencing primer sequence (e.g., for sequencing primers like those commercially available from Illumina or Element Biosciences). Alternatively, in some aspects, other sequences are used with a corresponding sequence primer, e.g., a custom sequencing primer, in place of a standard sequencing primer mixture.

In some aspects, the adapter comprises sequencing a primer sequence proximal to the barcode. Without wishing to be bound by theory, it is hypothesized that the proximal positioning of the sequencing primer and the barcode provides two main benefits. First, because the sequencing read (e.g., using Illumina or Element Biosciences sequencing) begins with the sequence directly downstream of the sequencing primer sequence, the barcode sequence is always located at the beginning of one of the two paired-end sequencing reads (e.g., from Illumina or Element Biosciences). After the barcode sequence, the sequencing read continues directly into an unknown region derived from the middle of the target molecule. Therefore, the proximal positioning of the barcode and sequencing primer ensures that the random barcode is easily identifiable, and avoids wasting sequencing capacity, e.g., time and resources, by repeatedly sequencing the region on the upstream side of the barcode (which is always derived from the end of the original target molecule). Second, the presence of a primer sequence (e.g., a primer from Illumina or Element Biosciences) adjacent to the barcode sequence provides a simple way to distinguish nucleic acid fragments containing barcodes from fragments that do not contain barcodes. In some aspects, these latter fragments arise when a copy of the amplified target molecule is broken more than once, thereby creating two end fragments with barcode sequences and one or more middle fragments without barcodes. In these instances, sequencing barcode-free fragments wastes sequencing capacity, e.g., time and resources, because they contain no barcode sequence to link them to a parent nucleic acid molecule. In some aspects, only end fragments containing barcode sequences contain the primer sequences (e.g., a primer from Illumina or Element Biosciences) that are used to selectively amplify these sequences by PCR.

In some aspects, an asymmetric adapter is ligated to both ends of a nucleic acid fragment (see FIG. 3). In some aspects, this ligation of an asymmetric adapter takes place following fragmentation, circularization, and shearing. In some aspects, this asymmetric adapter comprises two oligonucleotides, one of which is longer than the other. In some aspects, the shorter oligonucleotide is complementary to the longer oligonucleotide and, upon annealing, creates a ligation-competent adapter with a 3′ dT-tail suitable for specific ligation to the A-tailed fragment. In some aspects, the adapter sequence is complementary to a PCR primer that adds a second sequencing primer sequence (e.g., a primer from Illumina or Element Biosciences) by overlap-extension PCR, but only the longer of the two oligonucleotides is long enough to productively anneal to this primer during PCR. As a result, following ligation of an asymmetric adapter to both ends of a fragment, each of the two strands of the fragment has an annealing-competent sequence at only one end. In some embodiments, the second PCR primer in the reaction anneals to the partial sequence (e.g., primer sequence from Illumina or Element Biosciences) contained within the fragment adjacent to the barcode. Accordingly, in some embodiments, only exponentially amplified PCR product is the desired nucleic acid fragment. In some embodiments, such exponentially amplified PCR product begins with one primer sequence (e.g., a primer from Illumina or Element Biosciences), followed by the barcode sequence and an unknown sequence from the center of the target molecule, and ends with the second primer sequence (e.g., a primer from Illumina or Element Biosciences). In some embodiments, fragments of about 500 bp (e.g., about 250 bp, about 300 bp, about 350 bp, about 400 bp, about 450 bp, about 500 bp, about 550 bp, about 600 bp, about 650 bp, about 700 bp, or about 750 bp) are converted into a library suitable for sequencing. In some embodiments, conversion into a library suitable for sequence comprises adding any requisite binding sequences (e.g., Illumina or Element Biosciences flowcell binding sequences) to the ends of the fragments.

In some aspects, library preparation is similar to library preparation carried out with commercially-available reagents (e.g., from Illumina). In some embodiments, the library preparation is done with forked or Y-shaped adapters that ensure that the PCR-amplified products all have adapter 1 on one end and adapter 2 on the other end); however, in the method of the disclosure one of the forks of the Y-shaped adapter is omitted because the fragments of interest already contain an annealing site for one of the two sequencing primers. Therefore, in some aspects, one primer anneals to the remaining fork, and the other primer anneals to a site in the interior of the fragment. In some aspects, therefore, sequences (e.g., Illumina) are used to ensure compatibility with standard sequencing reagents (e.g., Illumina reagents) used in the sequencing methods. In some aspects, therefore, sequencing is carried out using a number or variety of sets of sequences (e.g., TruSeq™ kit, TruSeq™ Small RNA kit, and the like), any of which are useful in various aspects described herein.

In some embodiments, library preparation comprises methods similar to those conducted for an Element Biosciences workflow (e.g., according to the manufacturer's instructions). Accordingly, in some embodiments, methods comprise any one or any combination of appending universal linear double-stranded adaptor sequences using enzymatic ligation, appending universal Y-shaped adaptors using enzymatic ligation, and/or appending universal adaptor sequences using tailed PCR primers.

In some aspects, an adapter comprises a region that is identical among all members of the adapter population and a degenerate barcode region that is unique to each member of the population. In general, a barcode comprises a nucleic acid sequence that when observed together with a polynucleotide serves as an identifier of the sample or molecule from which the polynucleotide was derived. As used herein, the term “barcode” refers to a nucleic acid sequence that allows some feature of a polynucleotide with which the barcode is associated to be identified. In some aspects, the feature of the polynucleotide to be identified is the sample or molecule from which the polynucleotide is derived. In some aspects, barcodes are at least 3 nucleotides in length, e.g., 3, 4, 5, 6, 7, 8, 9, 10, 11, 12, 13, 14, 15, 16, or more nucleotides. In some aspects, barcodes are shorter than 10 nucleotides in length, e.g., 10, 9, 8, 7, 6, 5, or 4 nucleotides in length. In some aspects, barcodes associated with some polynucleotides are of different lengths than barcodes associated with other polynucleotides. In general, barcodes are of sufficient length and comprise sequences that are sufficiently different to allow the identification of samples based on barcodes with which they are associated. In some aspects, a barcode, and the sample source with which it is associated, is identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some aspects, each barcode in a plurality of barcodes differs from every other barcode in the plurality by at least two nucleotide positions, for example, by at least 2, 3, 4, 5, 6, 7, 8, 9, 10, or more positions. In some aspects, both a first adapter and a second adapter comprise at least one of a plurality of barcode sequences. In some aspects, barcodes for second adapter oligonucleotides are selected independently from barcodes for first adapter oligonucleotides.

In some aspects, the tripartite adapter further comprises an index sequence to facilitate multiplexing of more than one sample for simultaneous preparation and sequencing. As opposed to the barcode region, the index region is not degenerate but defined, and a set of distinct oligonucleotides are synthesized such that each contain a different index sequence. In some embodiments, index sequences are long enough to uniquely distinguish them from one another. In some embodiments, index sequences are long enough to uniquely distinguish them even if one or more errors are made during sequencing. In some aspects, typical lengths for the index sequence are 2-8 bases, e.g., 2, 3, 4, 5, 6, 7, or 8 bases. In some aspects, the index sequence is located to one side or the other of the degenerate barcode region, i.e., between the two priming regions, and is read along with the barcode in a single or a paired-end read. In other aspects, the index sequence is 5′ of the sequencing primer region in the synthesized oligonucleotide and 3′ of an additional sequence that anneals to oligonucleotides attached to the sequencing flowcell (or that anneals to a primer that adds such a sequence during PCR). In such aspects, the adapter is designed to mimic the structure of a sequencing-ready molecule, and the index is read by a separate index read on a sequencing machine (e.g., a machine from Illumina or Element Biosciences).

In some aspects, as an alternative to downstream linkage of two distinct barcode sequences ligated to the two ends of the target molecule, both ends of the target molecule are tagged with the same barcode sequence.

In some aspects, a single circularization barcode adapter is ligated to the target molecule in lieu of two end adapters. In some aspects, the two ends of this adapter ligate to the two ends of the same target molecule to form a circular molecule.

In some aspects, the adapter contains a single barcode sequence, which is flanked in the 5′ direction on each strand by uracil bases (see FIG. 5). In some aspects, after circularization, the USER™ enzyme mix (Uracil-Specific Excision Reagent) Enzyme (NEB) excises uracils and breaks the phosphate backbone. The term “USER enzyme” as used herein refers to USER™ (NEB), which is a mixture of Uracil DNA glycosylase (UDG) and the DNA glycosylase-lyase Endonuclease VIII. In some embodiments, UDG catalyzes the excision of a uracil base, forming an abasic (apyrimidinic) site while leaving the phosphodiester backbone intact. In some embodiments, the lyase activity of Endonuclease VIII breaks the phosphodiester backbone at the 3′ and 5′ sides of the abasic site, e.g., so that base-free deoxyribose is released. In some embodiments, each strand is broken 5′ of the barcode sequence, opening the circular molecule into a linear molecule with 5′ single-stranded overhangs at each end that contain the same barcode sequence. In some aspects, extension of the 3′ ends by, e.g., Klenow exo-DNA polymerase, copies the barcode sequence at each end, creating a fully double-stranded DNA molecule with the same barcode sequence at both ends. In some embodiments, Klenow exo-DNA polymerase extension leaves single dA-tails, e.g., for use in ligating additional adapters containing sequences that serve as PCR primer annealing sites, e.g., for subsequent PCR amplification.

In some aspects, a single circularizing adapter that contains two double-stranded copies of the same barcode sequence is ligated to the target molecule (see FIG. 6). In some aspects, such an adapter is prepared by synthesizing an oligonucleotide containing a degenerate barcode region and a region that forms a self-priming hairpin, extending the self-primed 3′ end with DNA polymerase, nicking the newly double-stranded molecule with a nicking endonuclease at a site near the 5′ end of the original oligonucleotide, and extending the exposed 3′ end with a strand-displacing DNA polymerase. In some aspects, after circularizing ligation to a target molecule, the adapter is cut at a specific site between the two copies of the barcode by a restriction enzyme or a combination of USER™ enzyme and a nuclease that specifically digests single-stranded DNA, such as S1 nuclease or mung bean nuclease.

In some aspects, an adapter comprising more than one copy, e.g., two copies, of the same barcode is used. In some embodiments, after circularization around the adapter, USER™ enzyme or another nuclease breaks the adapter between the barcode copies, yielding a linear molecule with the same barcode at both ends. A schematic of this approach is set out in FIG. 6. In some aspects, simultaneous fragmentation and adapter addition are carried out. In particular aspects, this simultaneous process is carried out by the use of transposases, which are discussed herein below in more detail.

In some aspects, adapter oligonucleotides are any suitable length. In some aspects, the length of the adapter is at least sufficient to accommodate the one or more sequence elements of which the adapter comprises. In some aspects, adapters are about, less than about, or more than about 10, about 15, about 20, about 25, about 30, about 35, about 40, about 45, about 50, about 55, about 60, about 65, about 70, about 75, about 80, about 90, about 100, about 120, about 140, about 160, about 180, about 200, about 300, about 400, about 500, about 600, about 700, about 800, about 900, or more nucleotides in length. In more particular aspects, adapters are 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

Adapter attachment can be carried out in any suitable manner. In some aspects, an adapter is attached to each end of each member of the target library. In some aspects, an adapter is attached to only one end, e.g., a single end, of each member of the target library. In some aspects, an adapter is attached to the nucleic acid following end-repair and any of dT-tailing, dA-tailing, dG-tailing, or dC-tailing. In some embodiments, tailing can be performed by Klenow exo-polymerase or Taq polymerase to add a single tailing nucleotide, or by terminal transferase to add multiple tailing nucleotides. In some aspects, the adapter is attached by ligation. The term “ligation” as used herein, with respect to two polynucleotides, refers to the covalent attachment or joining of two separate polynucleotides to produce a single larger polynucleotide with a contiguous backbone. Methods for joining two polynucleotides include, for example and without limitation, enzymatic and non-enzymatic (e.g., chemical) methods. Non-limiting examples of ligation reactions that are non-enzymatic include the non-enzymatic ligation techniques described in U.S. Pat. Nos. 5,780,613 and 5,476,930, which are herein incorporated by reference. In some embodiments, an adapter oligonucleotide is joined to a target polynucleotide by a ligase, for example a DNA ligase or RNA ligase. Ligases, each having characterized reaction conditions include, without limitation NAD-dependent ligases including tRNA ligase, Taq DNA ligase, Thermusfliformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase 1, DNA ligase III, DNA ligase IV, and genetically engineered variants thereof.

In some aspects, an adapter is ligated to each end of each double-stranded fragment of the target library. In particular aspects, a first tripartite adapter comprising an outer PCR primer region, an inner sequencing primer region, and a central barcode region is attached to each end of a short, linear nucleic acid sequence of the fragment library to form multiple barcode-tagged fragments or sequences, wherein the first adapter attached at the one end comprises a different barcode than the first adapter attached at the other end.

In some aspects, the addition of adapters occurs in a mixed solution and does not require physical separation of the nucleic acid in order to add the adapter. Thus, in various aspects, adapters are added to up to a million or more nucleic acids.

In some aspects, ligation is between polynucleotides having hybridizable sequences, such as complementary overhangs. The term “complementary” as used herein refers to a nucleic acid sequence of bases that can form a double-stranded nucleic acid structure by matching base pairs. In some aspects, ligation is between polynucleotides comprising two blunt ends. In some aspects, a 5′ phosphate is utilized in a ligation reaction. In some aspects, a 5′ phosphate is provided by the target polynucleotide, the adapter oligonucleotide, or both. In some aspects, 5′ phosphates are added to or removed from polynucleotides to be joined, as needed. Methods for the addition or removal of 5′ phosphates include, for example and without limitation, enzymatic and chemical processes. Enzymes useful in the addition and/or removal of 5′ phosphates include, but are not limited to, kinases, phosphatases, and polymerases.

### Nucleic Acid Amplification and Amplification Bias

In some embodiments, adapter-tagged target molecules are amplified using any suitable amplification method. “Amplification” as used herein refers to production of additional copies of a nucleic acid sequence, and can be carried out using PCR or any other suitable amplification technology (see, e.g., Dieffenbach and Dveksler, PCR Primer, a Laboratory Manual, Cold Spring Harbor Press, Plainview, N.Y. [1995]). Examples of suitable nucleic acid amplification methods include, but are not limited to, PCR, quantitative PCR, quantitative fluorescent PCR (QF-PCR), multiplex fluorescent PCR (MF-PCR), real time PCR (RT-PCR), single cell PCR, restriction fragment length polymorphism PCR (PCR-RFLP), PCK-RFLPIRT-PCR-IRFLP, hot start PCR, nested PCR, in situ polony PCR, in situ rolling circle amplification (RCA), bridge PCR, picotiter PCR, and emulsion PCR. Other suitable amplification methods include, but are not limited to, ligase chain reaction (LCR), transcription amplification, self-sustained sequence replication, selective amplification of target nucleic acids, consensus sequence primed polymerase chain reaction (CP-PCR), arbitrarily primed polymerase chain reaction (AP-PCR), degenerate oligonucleotide-primed PCR (DOP-PCR), and nucleic acid-based sequence amplification (NABSA).

In some aspects, PfuCx Turbo DNA polymerase (Agilent Technologies, La Jolla, CA) or KAPA HiFi Uracil+DNA Polymerase (Kapa Biosystems, Inc., Wilmington, MA) is used for PCR. In some embodiments, these polymerase enzymes are compatible with uracil-containing primers, yet feature a proofreading activity that reduces the error rate relative to Taq polymerases. In some aspects, polymerase mixtures optimized for “long-range” PCR are used. In some embodiments, these polymerase mixtures usually contain a mixture of Taq polymerase with a proof-reading polymerase. Non-limiting examples include LongAmp® Taq (NEB™) and MasterAmp™ Extra-long (Epicentre Bio). In some aspects, a single primer is used for PCR. It is contemplated herein that using a single primer discourages the accumulation of primer dimers during PCR (see, for example, Brown et al., Nucleic Acids Research, 1997, 26(16):3235-3241). In some other aspects, two or more primers are used for PCR.

In some aspects, PCR bias or “amplification bias” can be a significant challenge when amplifying complex, heterogeneous libraries that result from shearing genomic DNA. In some aspects, each barcode-tagged sequence in the library is amplified to a similar extent. In some aspects, if a subset of the target molecules dominate the PCR, fragments derived from those molecules are sequenced disproportionately frequently, and the yield of the sequencing reaction suffers. In some aspects, while some level of amplification bias is unavoidable, steps are taken to minimize impact of amplification bias. In some aspects, bias is minimized by supplementing the PCR reaction, e.g., with betaine, DMSO, or other known additive(s), or combinations thereof, to reduce the sequence dependence of amplification efficiency, promoting a more even distribution of amplified products.

In some aspects, PCR suppression effects are minimized. In some aspects, an identical sequence is ligated at both ends of a nucleic acid. In some aspects, upon denaturation during PCR, complementary ends anneal to form a hairpin, potentially reducing the efficiency of PCR. In some aspects, ligating the same adapter to both ends of the target molecule results in identical PCR primer-annealing, and primer-annealing sequences (e.g., Illumina primer-annealing sequences) contribute to PCR suppression hairpins, particularly when the two random barcode sequences in the adapters happen to be partially complementary. Illumina™ provides primer mixes with their sequencing reagent kits that include sequencing primers compatible with all of their various sequencing preparation kits. For example, multiple sequencing kits, each with their own sequences, are available from Illumina, and the primer mixture contains primers compatible with all of the kits.

Accordingly, in some embodiments, to minimize this effect, distinct PCR primer-annealing sequences and/or distinct primer-annealing sequences (e.g., Illumina) are included in the adapters that are attached to the two ends of the target molecule. In various aspects, steps are taken to avoid having identical adapters on both ends of the DNA, because when the DNA becomes single-stranded the ends can anneal to form a “panhandle” structure that blocks PCR primer annealing. In some aspects, this addition of primer annealing sequences is accomplished by adding a mixture of different adapters into the ligation mixture (in which case 1/n of the ligation products will have the same adapter on both ends, where n is the number of distinct adapters in the mixture). In other aspects, PCR suppression is promoted by the use of longer adapters in order to suppress amplification of shorter fragments in favor of longer fragments.

In some aspects, a “forked” or “Y” adapter comprising two oligonucleotides that are only partially complementary is used. In some aspects, such oligonucleotides anneal to form an adapter that is double stranded and ligation competent at one end, but forks into two non-complementary single strands at the other end. This type of adapter is often used in standard sequencing methods (e.g., Illumina sequencing methods) and may be used in some aspects of the disclosure. It is contemplated herein that a benefit of such a method is that subsequent PCR with primers complementary to the two strands yields products with one of the two fork sequences at one end and the other fork sequence at the other end, which is otherwise not possible at about 100% efficiency when ligating adapters to a library of unknown sequences. Standard sequencing protocols (e.g., Illumina) generally use a mixture of sequencing primers that contains primers compatible with different library preparation kits. In some embodiments, two primer mixtures are used: a “universal” primer mix that produces the first read, and an “index” primer mix that produces the second or paired end read. Therefore, by ligating two distinct universal primer-annealing sequences or two distinct index primer-annealing sequences to the target, PCR suppression hairpins can be avoided while preserving the ability of fragments derived from each end to be sequenced with the same standard (e.g., Illumina) primer mixture.

In some aspects, amplification bias is reduced by a linear amplification stage prior to exponential amplification (see FIG. 4). In some embodiments, thus, during the linear PCR amplification phase, only the initially present (original) molecules, and not the newly synthesized copies, are copied by PCR. In some aspects, the copying of only the original nucleic acid molecules is accomplished by ligating barcode-containing adapters with 3′ overhangs to the ends of the target molecule, such that only one of the two strands at each end of the ligated target molecule is capable of annealing to a PCR primer at a set annealing temperature. In some aspects, exponential amplification is triggered by a change in the annealing temperature or the addition of a nested primer.

In some aspects, amplification bias is minimized by replacing PCR with rolling-circle amplification (RCA) or hyperbranching rolling-circle amplification (HRCA). HRCA has been used in whole-genome amplification techniques known in the art and has been shown to amplify mixed populations with less bias than PCR. In some aspects, a circularization adapter is ligated to the target, such that the two ends of the adapter ligate to the ends of the same target molecule to form a circular molecule. In some aspects, the adapter contains a single barcode sequence, which is flanked in the 5′ direction on each strand by nicking endonuclease recognition sequences. In some aspects, after circularization, HRCA amplifies the molecule in an exponential manner. In some aspects, the resulting double-stranded DNA concatemers are broken, for example, by mechanical shearing or dsDNA fragmentase. In some aspects, the broken nucleic acids are then treated with a nicking endonuclease, which introduces single-strand breaks on each side of the barcode. In some aspects, each strand of the barcoded section becomes a 5′ overhang at the end of the resulting fragments, and a polymerase, e.g., Klenow, is used to fill in these ends, copying the barcode to create a blunt end ready for circularization.

In some aspects, two loop adapters are ligated to the ends of the target to create a circular “dumbbell” structure that is amplified by HRCA. In some embodiments, resulting concatemers are sheared and digested by a nicking endonuclease.

In some aspects, in place of mechanical or enzymatic fragmentation, random fragments are generated during amplification by PCR or rolling-circle amplification with random (degenerate) or partially random oligonucleotide primers (e.g., see FIG. 8A and FIG. 8B).

In some aspects, interior regions of the amplified target molecule are exposed prior to circularization by fragmentation using a double-stranded DNA fragmentase enzyme mixture (NEB). This enzyme mixture comprises two enzymes that create random breaks in double-stranded DNA. In some aspects, KAPA Frag Enzyme is used for fragmentation. Unlike exonucleases, fragmentation enzymes preserve both ends of the DNA molecule, both of which give rise to productive circular molecules. Unlike mechanical shearing, fragmentation enzymes introduce breaks along the length of the DNA molecule independent of the distance from an end of the molecule or the size of the molecule. Additionally, in some aspects, the number of breaks per kilobase is adjusted for different target molecule lengths by diluting the enzyme mixture or adjusting the reaction time. In some embodiments, reaction time takes about 15 minutes, but is adjusted accordingly, depending on the amount of DNA, the length of the DNA, and the concentration of the enzyme. The skilled person will recognize that reaction time is varied to achieve a desired goal of one break per DNA molecule and will appreciate the conditions necessary to achieve such a goal.

In some aspects, adapter-tagged target molecules are amplified by PCR using a single, uracil-containing oligonucleotide primer that is complementary to a constant region of the adapter lying outside of the barcode sequence, such that the barcode is copied by the extension of the primer. In some aspects, amplification creates many copies of each target molecule such that each copy of the same target molecule is attached to the same barcode sequence unique to that target molecule. In some aspects, the PCR primer sequence is removed from the end of each nucleic acid target molecule. In some aspects, the PCR primer sequence is removed by digestion with a USER™ enzyme, followed by end blunting, e.g., with Klenow fragment polymerase and/or T4 DNA polymerase.

In some aspects, amplified copies of the target molecules are randomly fragmented to create molecules with a barcode sequence at one end and a region of unknown sequence at the other end. In some aspects, the fragmented nucleic acid molecules are end-repaired to create blunt ends. In some aspects, biotinylated nucleotides are incorporated into the repaired ends. In some aspects, the fragmented nucleic acid molecules are circularized. In some aspects, circularizing the fragmented molecules is carried out by blunt-end ligation to bring the barcode sequence into proximity with the unknown region of sequence from the interior of the original target molecule. In some aspects, the circularized molecules are fragmented to create linear molecules. In some aspects, biotinylated molecules are attached to streptavidin-coated beads to facilitate handling and purification. In some aspects, an asymmetric adapter is ligated to each end of the linear molecules.

In some aspects, adapter-ligated fragments are amplified or copied. In some aspects, amplification is carried out by PCR using two oligonucleotide primers, the first of which is complementary to a constant sequence from the barcode-containing adapter, and the second of which is complementary to the overhanging sequence of the asymmetric adapter, and which together add sequences necessary for sequencing.

### Circularization and Fragmentation

In some aspects, fragmented nucleic acids are circularized. Circularization of a nucleic acid can be carried out in any suitable manner as known in the art. In some aspects, circularization is carried out by blunt-end ligation. In some aspects, this approach is used to minimize the intervening sequence between the barcode sequence and the unknown sequence region. In various aspects, sequencing such intervening sequence(s) in every sequencing read wastes capacity and decreases efficiency. In some aspects, the efficiency of blunt-end ligation circularization is low, particularly for long DNA molecules. In some aspects, circularization efficiency is improved, including by the use of a bridging oligonucleotide or adapter, by the creation of complementary sticky ends at the ends of the fragment, or by the use of recombinases (see, e.g., Peng et al., PLoS One 7(1): e29437, 2012).

In some aspects, a circularization adapter is used to circularize fragmented PCR copies that already have been barcoded. In some aspects, the circularized molecule is amplified by PCR. In some aspects, the circularized molecule is amplified by RCA.

In some aspects, barcode-tagged fragments comprising the barcode region at one end and a region of unknown sequence from an interior portion of the target nucleotide sequence at the other end are circularized, thereby bringing the barcode region into proximity with the region of unknown sequence.

Fragmentation (or fragmenting) of nucleic acid molecules is carried out in various aspects of the disclosure. For example, in some aspects, the methods of the disclosure comprise multiple fragmenting steps. Fragmenting of nucleic acids can be carried out by any suitable method known in the art. In some aspects, the circularized, barcode-tagged nucleic acid molecules are fragmented into linear fragments, some of which contain barcodes.

In some aspects, fragmenting of the circularized molecules is carried out by an acoustic shearing device (e.g., Covaris S2), and/or by Nextera™ transposases (Epicentre, Madison, WI) to combine shearing and the addition of asymmetric adapters. In some aspects, transposase technology, such as that used in the Nextera™ system (Epicentre), streamlines processing because transposases simultaneously fragment DNA and introduce adapter sequences at the newly exposed ends. Thus, transposases, in various aspects, replace fragmentation or shearing, end repair, end tailing, and adapter ligation with a single step. In some aspects, therefore, transposases are used in fragmentation. For example, in some aspects, transposes are used, e.g., for (1) fragmentation of genomic or other extremely large DNA molecules into target fragments 1-20 kb in length with concomitant attachment of tripartite adapters; (2) fragmentation of long target fragments with optional concomitant attachment of adapters designed to improve circularization efficiency; and/or (3) fragmentation of circularized DNA with concomitant attachment of asymmetric adapters. Accordingly, in some aspects, transposases are used to decrease the time necessary to prepare DNA samples for sequencing.

### Sequencing and Sequence Assembly

Various embodiments described herein relate to methods using high-throughput sequencing. In some aspects, the term “bulk sequencing,” “massively parallel sequencing,” or “next-generation sequencing (NGS)” refers to any high-throughput sequencing technology that parallelizes the DNA sequencing process. For example, in some aspects, bulk sequencing methods are typically capable of producing more than one million nucleic acid sequence reads in a single assay. In some aspects, the terms “bulk sequencing,” “massively parallel sequencing,” and “NGS” refer only to general methods, not necessarily to the acquisition of greater than one million sequence tags in a single run.

In some aspects, sequencing is carried out on any suitable sequencing platform, such as reversible terminator chemistry (e.g., Illumina), pyrosequencing using polony emulsion droplets, e.g., 454 sequencing (e.g., Roche), ion semiconductor sequencing (Ion Torrent™, Life Technologies), single molecule sequencing (e.g., SMRT, Pacific Biosciences, Menlo Park, CA), SOLiD sequencing (Applied Biosystems), sequencing-by-avidity (e.g., Element Biosciences), massively parallel signature sequencing, and the like.

Various embodiments described herein relate to methods of generating overlapping sequence reads and assembling them into a contiguous nucleotide sequence (“contig”) of a nucleic acid of interest. In some aspects, assembly algorithms align and merge overlapping sequence reads generated by methods described herein to provide a contiguous sequence of a nucleic acid of interest. In some aspects, nucleic acid sequence reads sharing the same barcode sequences are identified and grouped. In some aspects, each group of reads (i.e., grouped by a shared barcode sequence) is assembled into one or more longer contiguous sequences.

In some aspects, grouping of sequences is carried out by a computer program. For example, in various aspects, numerous sequence assembly algorithms or sequence assemblers are utilized, taking into account the type and complexity of the nucleic acid of interest to be sequenced (e.g., genomic DNA, PCR product, plasmid, and the like), the number and/or length of nucleic acids or other overlapping regions generated, the type of sequencing methodology performed, the read lengths generated, whether assembly is de novo assembly of a previously unknown sequence or mapping assembly against a reference sequence, and the like. In additional aspects, an appropriate data analysis tool is selected based on the function desired, such as alignment of sequence reads, base-calling and/or polymorphism detection, de novo assembly, assembly from paired or unpaired reads, or genome browsing and annotation.

In some aspects, overlapping sequence reads are assembled into contigs or the full or partial contiguous sequence of the nucleic acid of interest by sequence alignment, computationally or manually, whether by pairwise alignment or multiple sequence alignment of overlapping sequence reads.

In some aspects, overlapping sequence reads are assembled by sequence assemblers including, but not limited to ABySS, AMOS, Arachne WGA, CAP3, PCAP, Celera WGA Assembler/CABOG, CLC Genomics Workbench, CodonCode Aligner, Euler, Euler-sr, Forge, Geneious, MIRA, miraEST, NextGENe, Newbler, Phrap, TIGR Assembler, Sequencher, SeqMan NGen, SHARCGS, SSAKE, Staden gap4 package, VCAKE, Phusion assembler, Quality Value Guided SRA (QSRA), Velvet (algorithm) (Zerbino et al., Genome Res. 18(5): 821-9, 2008), SPAdes (http://bioinf.spbau.ru/spades), and the like.

In certain aspects, algorithms suited for short-read sequence data may be used including, but not limited to, Cross_match, ELAND, Exonerate, MAQ, Mosaik, RMAP, SHRiMP, SOAP, SSAHA2, SXOligoSearch, ALLPATHS, Edena, Euler-SR, SHARCGS, SHRAP, SSAKE, VCAKE, Velvet, PyroBayes, PbShort, and ssahaSNP.

In some aspects, the methods provided herein provide for the assembly of a contig or full continuous sequence of the nucleic acid of interest at lengths in excess of about 1 kb, about 2 kb, about 3 kb, about 4 kb, about 5 kb, about 6 kb, about 7 kb, about 8 kb, about 9 kb, about 10 kb, about 11 kb, about 12 kb, about 13 kb, about 14 kb, about 15 kb, about 16 kb, about 17 kb, about 18 kb, about 19 kb, about 20 kb, about 25 kb, about 30 kb, about 35 kb, about 40 kb, about 45 kb, or about 50 kb. In certain aspects, the methods provided herein provide for the assembly of a target nucleic acid with a length of about 0.1 kb, about 0.2 kb, about 0.3 kb, about 0.4 kb, about 0.5 kb, about 0.6 kb, about 0.7 kb, about 0.8 kb, about 0.9 kb, about 1.0 kb, about 1.1 kb, about 1.2 kb, about 1.3 kb, about 1.4 kb, about 1.5 kb, about 1.6 kb, about 1.7 kb, about 1.8 kb, about 2.0 kb, about 2.1 kb, about 2.2 kb, about 2.3 kb, about 2.4 kb, about 2.5 kb, about 2.6 kb, about 2.7 kb, about 2.8 kb, about 2.9 kb, about 3.0 kb, about 3.1 kb, about 3.2 kb, about 3.3 kb, about 3.4 kb, about 3.5 kb, about 3.6 kb, about 3.7 kb, about 3.8 kb, about 3.9 kb, about 4.0 kb, about 4.1 kb, about 4.2 kb, about 4.3 kb, about 4.4 kb, about 4.5 kb, about 4.6 kb, about 4.7 kb, about 4.8 kb, about 4.9 kb, about 5.0 kb, about 5.2 kb, about 5.3 kb, about 5.4 kb, about 5.5 kb, about 5.6 kb, about 5.7 kb, about 5.8 kb, about 5.9 kb, about 6.0 kb, about 6.1 kb, about 6.2 kb, about 6.3 kb, about 6.4 kb, about 6.5 kb, about 6.6 kb, about 6.7 kb, about 6.8 kb, about 6.9 kb, about 7.0 kb, about 7.1 kb, about 7.2 kb, about 7.3 kb, about 7.4 kb, about 7.5 kb, about 7.6 kb, about 7.7 kb, about 7.8 kb, about 7.9 kb, about 8.0 kb, about 8.1 kb, about 8.2 kb, about 8.3 kb, about 8.4 kb, about 8.5 kb, about 8.6 kb, about 8.7 kb, about 8.8 kb, about 8.9 kb, about 9.0 kb, about 9.1 kb, about 9.2 kb, about 9.3 kb, about 9.4 kb, about 9.5 kb, about 9.6 kb, about 9.7 kb, about 9.8 kb, about 9.9 kb, about 10.0 kb, about 10.5 kb, about 11.0 kb, about 11.5 kb, about 12.0 kb, about 12.5 kb, about 13.0 kb, about 13.5 kb, about 14.0 kb, about 14.5 kb, about 15.0 kb, about 15.5 kb, about 16.0 kb, about 16.5 kb, about 17.0 kb, about 17.5 kb, about 18.0 kb, about 18.5 kb, about 19.0 kb, about 19.5 kb, about 20.0 kb, about 20.5 kb, about 21.0 kb, about 21.5 kb, about 22.0 kb, about 22.5 kb, about 23.0 kb, about 23.5 kb, about 24.0 kb, about 24.5 kb, about 25.0 kb, about 30.0 kb, about 35.0 kb, about 40.0 kb, about 45.0 kb, about 50.0 kb, about 55.0 kb, about 60.0 kb, about 65.0 kb, about 70.0 kb, about 75.0 kb, about 80.0 kb, about 85.0 kb, about 90.0 kb, about 95.0 kb, or about 100 kb, or greater.

Alternatively, in some aspects, the methods provided herein provide for the assembly of a contig or full continuous sequence of the nucleic acid of interest at lengths of less than about 1 kb, about 900 bp, about 800 bp, about 700 bp, about 600 bp, or about 500 bp, or less.

In some aspects, the methods provided herein provide for the assembly of a contig or full continuous sequence of the nucleic acid of interest with very high per base accuracy or fidelity. The term “accuracy” or “fidelity” as used herein refers to the degree to which the measurement conforms to the correct, actual, or true value of the measurement. For example, in some aspects, accuracy or fidelity of the disclosed method is greater than about 80%, about 90%, about 95%, about 99%, about 99.5%, about 99.9%, about 99.95%, about 99.99%, about 99.999%, or greater. In some aspects, sequencing errors affecting per base and average accuracy of sequence information due to the underlying sequencing platform are substantially or completely corrected by majority calls by the assembly methods and systems described herein, e.g., such as a computer acting as an assembler. In some aspects, an output with a single long read is produced from putting together multiple long reads.

In particular aspects, the methods provided herein provide for the assembly of the nucleic acid of interest with about 100% accuracy, about 99.99% accuracy, about 99.98% accuracy, about 99.97% accuracy, about 99.96% accuracy, about 99.95% accuracy, about 99.94% accuracy, about 99.93% accuracy, about 99.92% accuracy, about 99.91% accuracy, about 99.90% accuracy, about 98.99% accuracy, about 98.98% accuracy, about 98.97% accuracy, about 98.96% accuracy, about 98.95% accuracy, about 98.94% accuracy, about 98.93% accuracy, about 98.92% accuracy, about 98.91% accuracy, about 98.90% accuracy, about 98.89% accuracy, about 98.88% accuracy, about 98.87% accuracy, about 98.86% accuracy, about 98.85% accuracy, about 98.84% accuracy, about 98.83% accuracy, about 98.82% accuracy, about 98.81% accuracy, about 98.80% accuracy, about 98.79% accuracy, about 98.78% accuracy, about 98.77% accuracy, about 98.76% accuracy, about 98.75% accuracy, about 98.74% accuracy, about 98.73% accuracy, about 98.72% accuracy, about 98.71% accuracy, about 98.70% accuracy, about 98.69% accuracy, about 98.68% accuracy, about 98.67% accuracy, about 98.66% accuracy, about 98.65% accuracy, about 98.64% accuracy, about 98.63% accuracy, about 98.62% accuracy, about 98.61% accuracy, about 98.60% accuracy, about 98.5% accuracy, about 98.0% accuracy, about 97.5% accuracy, about 97.0% accuracy, about 96.5% accuracy, about 96.0% accuracy, about 9 5.5% accuracy, about 95.0% accuracy, about 94.5% accuracy, about 94.0% accuracy, about 93.5% accuracy, about 93.0% accuracy, about 92.5% accuracy, about 92.0% accuracy, about 9 1.5% accuracy, about 91.0% accuracy, about 9 0.5% accuracy, about 9 0.0% accuracy, about 89.% accuracy, about 88% accuracy, about 87% accuracy, about 86% accuracy, about 85% accuracy, about 84% accuracy, about 83% accuracy, about 82% accuracy, about 81% accuracy, or about 80% accuracy.

In some aspects, the methods provided herein provide for the assembly of a contig or full continuous sequence of the nucleic acid of interest with an error rate of about 0.001%, about 0.002%, about 0.003%, about 0.004%, about 0.005%, about 0.006%, about 0.007%, about 0.008%, about 0.009%, about 0.010%, about 0.011%, about 0.012%, about 0.013%, about 0.014%, about 0.015%, about 0.016%, about 0.017%, about 0.018%, about 0.019%, about 0.020%, about 0.025%, about 0.030%, about 0.035%, about 0.040%, about 0.045%, about 0.050%, about 0.055%, about 0.060%, about 0.065%, about 0.070%, about 0.075%, about 0.080%, about 0.085%, about 0.090%, about 0.095%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.5%, about 6.0%, about 6.5%, about 7.0%, about 7.5%, about 8.0%, about 8.5%, about 9.0%, about 9.5%, about 10.0%, about 15%, or about 20%.

In some aspects, the methods described herein take less than 5 days, less than 4 days, less than 3 days, less than 2 days, or less than 1 day. In particular aspects, the methods described herein take about 3 days, because the methods comprise elements that run overnight (i.e., PCR amplification and ligation). In some aspects, the methods are shortened (or sped up) by the use of faster PCR thermocyclers and faster polymerases, and/or by using higher concentrations of ligase. Such improvements, in some aspects, shorten the protocol to about two days. Further improvements, including the use of Nextera™ transposon, as described above, also eliminate protocol components, speeds up the protocol, and shortens overall method time.

In some aspects, the methods described herein are much simpler and more convenient than other methods. For example, in some aspects, the methods of the disclosure are carried out in a single tube, thus involving less handling, and eliminating the need to split the library into multiple-well plates.

In some aspects, the methods of the disclosure facilitate haplotyping of chromosomes of polyploid species. A “haplotype” is a collection of specific alleles (e.g., particular DNA sequences) in a cluster of tightly-linked genes on a chromosome that are likely to be inherited together. In other words, a “haplotype” is the group of genes that a progeny inherits from one parent. A cell or a species is “polyploid” if it contains more than two haploid (n) sets of chromosomes. In other words, the chromosome number for the cell or species is some multiple of n greater than the 2n content of diploid cells. For example, triploid (3n) and tetraploid cell (4n) cells are polyploid. In some aspects, the methods of the disclosure are useful in haplotype reconstruction from sequence data, or by haplotype assembly.

### Methods of the Disclosure

For example, in one example embodiment, fragments of nucleic acid are assembled into distinct nucleic acid sequences by fragmenting a target nucleic acid molecule and attaching the same random nucleic acid barcode to each short sequencing-ready nucleic acid fragment that derives from the nucleic acid molecule. In some embodiments, to each end of each fragment in the starting library is ligated a first “tripartite” adapter comprising an outer PCR annealing region, a central random barcode sequence, and an inner sequencing primer region. In some embodiments, the adapter-ligated library is then diluted, and about one million molecules are amplified by PCR using a primer complementary to the PCR annealing region on the adapter. In certain embodiments, fewer than one million molecules are amplified by PCR, e.g., fewer than 100,000, fewer than 150,000, fewer than 200,000, fewer than 250,000, fewer than 300,000, fewer than 350,000, fewer than 400,000, fewer than 450,000, fewer than 500,000, or fewer than 750,000. In certain embodiments, more than one million molecules are amplified by PCR, e.g., more than 1,100,000, more than 1,200,000, more than 1,300,000, more than 1,400,000, more than 1,500,000, more than 1,750,000, or more than 2,000,000. In various aspects, the library is diluted by orders of magnitude greater or lesser than the million molecules, depending on the goal of the sequencing and the resources available. For example, the complexity depends upon the amount of sequencing and the length of the target. In some aspects, about 10,000 or more molecules are amplified; whereas, in some aspects about 1,000,000 or more molecules are amplified. In some aspects, dilution of the library ensures that enough reads are derived from each molecule to allow full assembly. In some embodiments, each of the about one million library sequences is copied many times with PCR. In some embodiments, the PCR annealing region is removed from each 5′ end of the amplified nucleic acid with USER™ enzyme, which cuts the DNA backbone at uracil bases designed into the PCR primer. In some embodiments, therefore, the barcode sequences are thus positioned at the ends of each molecule. In some embodiments, an enzyme mixture called dsDNA fragmentase is then used to randomly cut each copy in a different location. In some embodiments, the ends of the nucleic acid are repaired (blunted) in the presence of biotin-dCTP, which results in biotinylation of the ends of the nucleic acid molecules. In some aspects, dC nucleotides are designed into the tripartite adapter to ensure successful biotinylation. In some embodiments, the nucleic acid is then circularized, bringing the barcode sequence at one end into proximity with an unknown sequence region randomly selected from the length of the starting molecule. The circularized nucleic acid is again fragmented, this time by shearing (including, in some aspects, mechanical or acoustic shearing), to obtain molecules of a desired length. In some aspects, the desired nucleic acid length is about 300 bp to about 800 bp (e.g., about 300 bp, about 400 bp, about 500 bp, about 600 bp, about 700 bp, or about 800 bp), but this may be modified depending on the sequencing instrument used and the goals of the sequencing. In some aspects, the nucleic acid fragments containing the barcodes are bound to streptavidin-coated magnetic beads, end-repaired, dA-tailed, and ligated to another adapter. In some embodiments, this “second” adapter comprises two oligonucleotides of different lengths, such that when annealed the shorter oligonucleotide has a 3′ dT overhang and the longer oligonucleotide, which corresponds to a second sequencing primer annealing sequence, has a longer 3′ overhang. In some aspects, only the longer oligonucleotide (and not the subsequently synthesized reverse complement of the shorter adapter) is able to subsequently anneal to the PCR primer. In some embodiments, the beads are added to a PCR mixture containing primers that anneal to the two sequencing primer regions (one of which was added by the first adapter, the other by the second adapter). In some embodiments, PCR exponentially amplifies only the region of the template from the first sequencing primer, in the direction of the barcode and the sequence of interest, through the second adapter, and adds sequences that allow annealing to the sequencing flow cell. In some aspects, the resulting nucleic acid molecules are size-selected. In some aspects, size selection and, therefore, tighter size distribution, leads to better sequencing results.

In some embodiments, if size selection is performed, the size selection is carried out by the Agencourt AMPure XP system (Beckman Coulter, Brea, CA), or by gel purification. In some embodiments, the nucleic acid molecules are then sequenced, using a single-end read or paired-end reads. In some embodiments, the sequencing data from the first read contains the barcode sequence followed by sequence from the original fragment. In some aspects, it also is possible to switch the method so that the barcode is on the second read. In some embodiments, all sequences with identical barcodes are grouped, and each group is assembled into the full-length sequence independent of the others. In various aspects, this method is adapted for use on any of the available high-throughput sequencing platforms.

In a further aspect, the embodiment outlined above generates two barcode-defined groups of reads corresponding to each original target molecule, defined by the two distinct barcode sequences in the adapters that are ligated to the two ends of the target molecule. Each target molecule is thus “tagged” with two different barcode sequences. In some embodiments, fragments containing one of the two barcode sequences are pooled and assembled separately from those containing the other barcode sequence. In some aspects, the two barcode sequences are linked by a supplemental experimental preparation and/or computational analysis, allowing all reads containing either of the barcode sequences to be pooled and assembled together. In some aspects, the length of the target molecules that are sequenced is thereby doubled, the efficiency of the method is increased, and the problem of decreasing circularization efficiency with increasing molecule length is partially offset. In some aspects, a subset of the PCR-amplified, barcode adapter-ligated target molecules is not fragmented. In some aspects, a subset is physically separated from the fragmented population, and this separated fraction is not subjected to fragmentation. In other aspects, fragmentation of the population is incomplete, and those molecules that escape fragmentation are used for barcode linking. In some aspects, circularization of intact molecules brings the two barcode sequences ligated to that target molecule into proximity. In some aspects, the region containing the two barcode sequences is separated from the target molecule by PCR or restriction endonuclease digestion, converted into sequencing-ready molecules by the addition of appropriate adapter sequences, and sequenced in the same sequencing run as the main library or in a separate run. In some embodiments, in the bioinformatic processing pipeline, these linked barcode sequence pairs are identified, and groups of reads tagged with each of the barcode sequences are merged into a single group for assembly into the longer sequence.

In some aspects of the methods described herein, barcode sequences are linked. In some aspects, the linked barcode sequences allow the two barcode-defined groups of reads to be merged by circularizing a small percentage of the products of the first PCR amplification while forgoing fragmentation, such that the barcode sequences at each end are brought into proximity with one another. In some aspects, the circularized full-length molecules remain in the same mixture as the circularized fragmented molecules. In some aspects, both types of molecule are processed together and sequenced in the same sequencing reaction. In various aspects, sequencing reads capturing paired barcode sequences are identified computationally. In some aspects, when this approach is used, it is desirable to use a mixture of tripartite adapters containing distinct sequencing primer regions to avoid hairpin formation. Alternatively, forked adapters may be used so that the two ends of the target molecules receive different sequencing primer sequences. In some aspects, a portion of the circularized mixture is removed (before or after fragmentation) and used to prepare samples for barcode pairing. In some aspects, the circularized molecules (which may or may not have previously been fragmented to open the circles) are digested with a restriction endonuclease that recognizes a specific site in the constant regions of the barcode adapter. In one aspect, the restriction endonuclease SapIrecognizes a site in the sequence of the Illumina TruSeq adapter sequence. In some aspects, asymmetric adapters are ligated to the ends, e.g., newly exposed sticky end or ends. In some aspects, the adapter-ligated fragments are amplified by PCR using two oligonucleotide primers, the first of which is complementary to a constant sequence from the barcode-containing adapter, and the second of which is complementary to the overhanging sequence of the asymmetric adapter, and which together add sequences for sequencing on a sequencing instrument (e.g., Illumina™). In some aspects, forked or Y-shaped adapters are ligated to the newly exposed end or ends. In some aspects, the adapter-ligated fragments are amplified by PCR using two oligonucleotide primers, one of which is complementary to a sequence on one fork of the adapter and the other of which is complementary to a sequence on the second fork of the adapter. The type of adapters to be used depends on what barcode adapter design is used. In some aspects, the two barcode sequences are identified in the sequencing data. In some aspects, the two groups of reads in the primary sequencing data set defined by each of the linked barcodes are merged and assembled into longer sequences. In some aspects, the short constant sequences bordering the barcodes identify true barcode pairs from spurious sequences.

In a particular aspect, the disclosure provides a method for obtaining nucleic acid sequence information from a nucleic acid molecule by assembling a series of short nucleic acid sequences into longer nucleic acid sequences (i.e. intermediate or long nucleic acid sequences). In some aspects, the method comprises some, if not all, of fragmenting the nucleic acid molecule comprising a nucleic acid sequence or a genomic nucleic acid sequence into a plurality of linear nucleic acid sequences; attaching a first adapter to the linear nucleic acid sequence, the first adapter comprising an outer polymerase chain reaction (PCR) primer region (or nucleic acid amplification region), an inner sequencing primer region, and a central barcode region to each end of the linear nucleic acid sequences to form barcode-tagged sequences, wherein the first adapter attached at one end comprises a different barcode than the first adapter attached at the other end; replicating the barcode-tagged sequences, e.g., by PCR, to obtain a library of barcode-tagged sequences using a primer complementary to the PCR primer region; removing the PCR primer region from the barcode-tagged sequences; breaking the barcode-tagged sequences at random locations using an enzyme that generates linear, barcode-tagged fragments comprising the barcode region at one end and a region of unknown sequence at the other end; circularizing the linear, barcode-tagged fragments comprising the barcode region at one end and a region of unknown sequence from an interior portion of the target nucleotide sequence at the other end, thereby bringing the barcode region into proximity with the region of unknown sequence; fragmenting the circularized, barcode-tagged fragments into linear, barcode-tagged fragments; attaching a second adapter comprising two oligonucleotides of different lengths to each end of the linear, barcode-tagged fragments to form double adapter-ligated barcode-tagged nucleic acid fragments, wherein one end of the second adapter is double stranded to facilitate ligation and the other end of the second adapter comprises a 3′ single-stranded overhang, and wherein only the longer of the two oligonucleotides comprises a sequence complementary to a second sequencing primer and comprises sufficient length to allow annealing of that primer; replicating the double adapter-ligated barcode-tagged nucleic acid fragments by PCR using two primers, the first of which is complementary to a constant sequence from the barcode-containing adapter, and the second of which is complementary to the overhanging sequence of the asymmetric adapter, and which together add sequences necessary for nucleic acid sequencing; sequencing the double adapter-ligated barcode-tagged nucleic acid fragments beginning with the barcode region followed by the target sequence; sorting a series of sequenced nucleic acid fragments into independent groups based on shared barcodes; and assembling each group of short nucleic acids into one or more longer nucleic acid sequences, independent of all other groups.

### Sample Preparation

In some example aspects of the disclosure, nucleic acid samples are prepared as described below. Only one strand of the nucleic acid is described and set out below.

(1) A tripartite adapter is ligated to the end of the target molecule:

Ligated target-
(SEQ ID NO: 46)
NNNCCTACACGACGCTCTTCCGATCTNNNNNNNNNNNNNNNNCCAGGAA
TAGTTATGTGCATTAATGAATGGCGCC

(2) Target molecules with adapters at both ends are amplified and the PCR primer annealing region (i.e., the region after “ . . . NNNNCC”) is removed:

Ligated target-
(SEQ ID NO: 47)
NNNCCTACACGACGCTCTTCCGATCTNNNNNNNNNNNNNNNNCC

(3) Amplified target molecules are fragmented and circularized:

Ligated target end-
(SEQ ID NO: 47)
NNNCCTACACGACGCTCTTCCGATCTNNNNNNNNNNNNNNNNCC-
ligated region of interest

(4) Circularized DNA is fragmented and fragments containing adapter sequences are prepared for sequencing:

Adapter 1 (e.g., Illumina)-
(SEQ ID NO: 52)
CCTACACGACGCTCTTCCGATCTNNNNNNNNNNNNNNNNCC-
ligated region of interest-Adapter 2
(e.g., Illumina)

(5) Resulting sequencing read:

NNNNNNNNNNNNNNNNCC-ligated region of interest

(6) In the computational pipeline, the sequences at the start of the read are used to determine the sample and target molecule of origin:

NNNNNNNNNNNNNNNNCC-ligated region of interest

The 5′ multiple N region determines the target molecule of origin. The “CC” region confirms the upstream sequence is a barcode. The 3′ region contains sequence information for the ligated region of interest.

### Sample Preparation for Barcode Pairing

In some aspects, samples are prepared for barcode pairing as described below. Only one strand of the nucleic acid is described and set out below.

(1) Tripartite adapter is ligated to the end of the target molecule:

Ligated target-
(SEQ ID NO: 46)
NNNCCTACACGACGCTCTTCCGATCTNNNNNNNNNNNNNNNNCCAGGAA
TAGTTATGTGCATTAATGAATGGCGCC

(2) Target molecules with adapters at both ends are amplified and the PCR primer annealing region (i.e., the region after “ . . . NNNNCC”):

Ligated target-
(SEQ ID NO: 47)
NNNCCTACACGACGCTCTTCCGATCTNNNNNNNNNNNNNNNNCC

(3) Full-length amplified target molecules that avoid fragmentation are circularized:

Ligated target end-
(SEQ ID NO: 48)
NNNCCTACACGACGCTCTTCCGATCTNNNNNNNNNNNNNNNNCC-
GGNNNNNNNNNNNNNNNNAGATCGGAAGAGCGTCGTGTAGGNNN-
Ligated target end

(4) Circularized DNA is fragmented and fragments containing adapter sequences are prepared for sequencing:

Adapter 1 (e.g. Illumina)-NNNNNNNNNNNNNNNNCC-
GGNNNNNNNNNNNNNNNN-Adapter 2 (e.g., Illumina)

(5) Resulting sequencing read:

NNNNNNNNNNNNNNNNCC-GGNNNNNNNNNNNNNNNN-Adapter 2
(e.g., Illumina)

(6) In the computational pipeline, the two barcodes (i.e., the multiple “Ns” set out below at each end of the sequence) are identified as a pair:

NNNNNNNNNNNNNNNNCC-GGNNNNNNNNNNNNNNNN

The 5′ and 3′ multiple N regions represent the paired barcodes.

### Multiplexed Sample Preparation

In some aspects, multiplexed samples are prepared as described below. Only one strand of the nucleic acid is described and set out below.

(1) Tripartite adapter is ligated to the end of the target molecule. Underlined, bolded font indicates the index sequence (e.g., ATCACG) unique to each sample:

Ligated target-
(SEQ ID NO: 49)
NNNCCTACACGACGCTCTTCCGATCTNNNNNNNNNNNNNNNNATCACGC
AGGAATAGTTATGTGCATTAATGAATGGCGCC

(2) Target molecules with adapters at both ends are amplified and the PCR primer annealing region (i.e., the region after “ . . . NNATCACGC”) is removed:

Ligated target--
(SEQ ID NO: 50)
NNNCCTACACGACGCTCTTCCGATCTNNNNNNNNNNNNNNNNATCACGC

(3) PCR products deriving from multiple samples are mixed and processed together in a single tube from this point. Each contains a unique index sequence. Amplified target molecules are fragmented and circularized:

Ligated target end-
(SEQ ID NO: 50)
NNNCCTACACGACGCTCTTCCGATCTNNNNNNNNNNNNNNNNATCACGC-
ligated region of interest

(4) Circularized DNA is fragmented and fragments containing adapter sequences are prepared for sequencing:

Adapter 1 (e.g., Illumina)-
(SEQ ID NO: 51)
CCTACACGACGCTCTTCCGATCTNNNNNNNNNNNNNNNNATCACGC-
ligated region of interest-Adapter 2
(e.g., Illumina)

(5) Resulting sequencing read:

NNNNNNNNNNNNNNNNATCACGC-ligated region of interest

(6) In the computational pipeline, the sequences at the start of the read are used to determine the sample and target molecule of origin:

NNNNNNNNNNNNNNNNATCACGCligated region of interest

The 5′ N region represents the barcode and determines the origin of the target molecule. The “ATCACG” region represents the index sequence and determines origin of the sample. The ligated region of interest contains the sequence information.

### Multiplexed Sample Preparation for Barcode Pairing

In some aspects, multiplexed samples are prepared for barcode pairing as described below. Only one strand of the nucleic acid is described and set out below.

(1) Tripartite adapter is ligated to the end of the target molecule. Underlined, bolded font indicates the index sequence (e.g., ATCACG) unique to each sample:

Ligated target-
(SEQ ID NO: 49)
NNNCCTACACGACGCTCTTCCGATCTNNNNNNNNNNNNNNNNATCACGC
AGGAATAGTTATGTGCATTAATGAATGGCGCC

(2) Target molecules with adapters at both ends are amplified and the PCR primer annealing region (i.e., the region after “ . . . NNATCACGC”) is removed:

Ligated target--
(SEQ ID NO: 53)
NNNCCTACACGACGCTCTTCCGATCTNNNNNNNNNNNNNNNN ATCACGC

(3) PCR products deriving from multiple samples are mixed and processed together in a single tube from this point. Each contains a unique index sequence (underlined font). Full-length amplified target molecules that avoid fragmentation are circularized:

Ligated target end--
(SEQ ID NO: 51)
NNNCCTACACGACGCTCTTCCGATCTNNNNNNNNNNNNNNNN ATCACGC-
GCGTGATNNNNNNNNNNNNNNNNAGATCGGAAGAGCGTCGTGTAGGNNN-
Ligated target end

(4) Circularized DNA is fragmented and fragments containing adapter sequences are prepared for sequencing:

Adapter 1 (e.g., Illumina)-NNNNNNNNNNNNNNNNATCACGC-
GCGTGATNNNNNNNNNNNNNNNN-Adapter 2 (e.g., Illumina)

(5) Resulting sequencing read:

NNNNNNNNNNNNNNNNATCACGC-
GCGTGATNNNNNNNNNNNNNNNN-Adapter 2 (e.g., Illumina)

(6) In the computational pipeline, the two barcodes are identified as a pair and the index determines the sample of origin. Matching indexes confirm intramolecular circularization:

NNNNNNNNNNNNNNNNATCACGC--
GCGTGATNNNNNNNNNNNNNNNN

The 5′ and 3′ multiple N regions represent the paired barcodes. The “ATCACG” region represents the index sequence and determines origin of sample. The “CGTGAT” sequence or region is the reverse complement of the first index sequence, confirming intramolecular circularization.

### Computational Pipeline and Sequence Assembly

In some aspects, once a library created according to the methods of the disclosure has been sequenced, the sequencing data is processed to assemble the raw short nucleic acid sequences (or short reads) into synthetic long nucleic acid sequences (long reads). In some embodiments, the “computational pipeline” or “processing pipeline” is as described below.

In some aspects, sequencing reads are trimmed to remove regions of low quality, as well as known adapter sequences. A number of open-source tools are available for this purpose including, but not limited to, Trimmomatic (http://www.usadellab.org/cms/?page=trimmomatic), Skewer (http://www.biomedcentral.com/1471-2105/15/182), the FASTX-toolkit (http://hannonlab.cshl.edu/fastx_toolkit/), Scythe (http://github.com/vsbuffalo/scythe), and others.

In some aspects, sequencing reads are searched for barcode sequences. In some aspects, the first sixteen bases of the read are identified as a barcode if the subsequent bases match the known constant region in the tripartite adapter, e.g., “CC.” In some embodiments, the barcode sequence, the constant sequence, and any other adapter sequences or fragments thereof (such as sequences left over from incomplete removal of the PCR primer region) are removed from the read. Accordingly, the remainder of the read constitutes sequence information from the molecule identified by the specific barcode. In some aspects, a hash table is created in which the barcode sequences are the keys and the sequence information is the values. That is, each distinct barcode defines a bin, and each sequence read is placed in the bin defined by its barcode. In some aspects, if paired-end reads are used, the reverse read is placed in the same bin as the forward read.

In some aspects, when barcode pairing data is available, those reads are analyzed to find paired barcodes. In some embodiments, after trimming adapters and low-quality regions, reads are inspected for the expected pattern, e.g., barcode 1, defined sequence 1, reverse complement of defined sequence 2, reverse complement of barcode 2, and adapter sequence. In some embodiments, barcode pairs are extracted from sequences matching this pattern. In some aspects, a data structure is created to count how many times each barcode is paired with other barcodes. Accordingly, a true pair is verified when two barcodes are paired with each other more times than a threshold number and more times than either is paired with any other barcode. In some embodiments, once a true pair is verified, the sequence read bins corresponding to the two barcodes are merged into a single bin for assembly.

In some aspects, the sequences in each barcode-defined bin are assembled into synthetic long reads. In such embodiments, each bin is assembled independently of the other bins, allowing parallelization of assembly. A number of open-source assemblers are available in the art, including those described herein above.

In one aspect, the present disclosure includes a computational pipeline for assembling grouped reads. In some embodiments, after quality checking each read for low confidence calls and for sequences matching the adapters used in the protocol, the first bases can be split from the read and defined as the barcode. In some embodiments, a hash table is built that groups the subset of reads associated with each barcode. In some embodiments, each group is then assembled individually, with or without a reference genome, using standard alignment and assembly software (e.g., Bowtie 2, Velvet, or SPAdes).

In some embodiments, the methods disclosed herein are used with nanopore sequencing platforms as described in U.S. Patent Publication Number 2014/0034497, which is herein incorporated by reference in its entirety. In some embodiments, the methods are used with Pacific Biosciences sequencing platforms as described in U.S. Pat. Nos. 7,315,019 and 8,652,779, which are each herein incorporated by reference in their entireties. In some embodiments, the methods are used with Illumina sequencing platforms as described in U.S. Pat. No. 7,115,400 and PCT Publication Number WO/2007/010252, which are herein incorporated by reference in their entirety. In some embodiments, the methods are used with IonTorrent™ sequencing platforms as described in PCT Patent Publication Number WO/2008/076406, which is herein incorporated by reference in its entirety. In some embodiments, the methods are used with Roche/454 sequencing platforms as described in U.S. Patent Number WO/2004/070005, which is herein incorporated by reference in its entirety.

In some embodiments, as illustrated in the examples below, the method comprises: (a) creating a target nucleic acid library (e.g., by mechanical shearing, PCR, restriction digestion, or another method); (b) preparing that library for adapter attachment (e.g., by end-repair and dT-tailing); (c) creating a mixture of adapter fragments (e.g., comprising regions that are identical among all members of the adapter population and a degenerate “barcode” region that is unique to each member of the population); (d) attaching one adapter to each end of each member of the target library (e.g., by ligation); (e) amplifying the adapter-ligated target molecules by PCR (e.g., using a single, uracil-containing oligonucleotide primer that is complementary to a constant region of the adapters lying 5′ of the barcode sequence, to create many copies of each target molecule such that each copy of the same target molecule is attached to the same barcode sequences unique to that target molecule); (f) optionally removing the PCR primer sequence from the 5′ end of each DNA strand (e.g., by digestion with USER™ enzyme); (g) randomly fragmenting the amplified copies of the targets (e.g., to create molecules with a barcode sequence at one end and a region of unknown sequence at the other end); (h) end-repairing the fragmented molecules (e.g., to create blunt ends while incorporating biotinylated nucleotides into the repaired ends); (i) circularizing the fragmented molecules (e.g., by blunt-end ligation to bring the barcode sequence into proximity with the unknown region of sequence from the interior of the original target molecule); (j) fragmenting the circularized molecules (e.g., to create linear molecules); (k) optionally attaching the biotinylated molecules to streptavidin-coated beads (e.g., to facilitate handling and purification); (1) ligating an asymmetric adapter to each end of the linear molecules; (m) amplifying the adapter-ligated fragments (e.g., by PCR using two oligonucleotide primers, the first of which is complementary to a constant sequence from the barcode-containing adapter, and the second of which is complementary to the overhanging sequence of the asymmetric adapter, and which together add sequences necessary for sequencing on a sequencing instrument); (n) sequencing the amplified DNA (e.g., on a massively parallel short-read instrument); (o) computationally identifying and grouping reads sharing the same barcode sequences; and (p) assembling each group of reads (e.g., defined by a shared barcode sequence into longer contiguous sequences describing the original target molecule).

In some embodiments of the method as outlined above, two barcode-defined groups of reads are generated corresponding to each original target molecule (e.g., defined by the two distinct barcode sequences in the adapters that ligated to the two ends of the target molecule). In some embodiments, each target molecule is tagged with two different barcode sequences. In some embodiments, fragments containing one of the two barcode sequences can be pooled and assembled separately from those containing the other barcode sequence. In some embodiments, the two barcode sequences are linked by a supplemental experimental preparation, allowing all reads containing either of the barcode sequences to be pooled and assembled together. In some embodiments, a subset of the PCR-amplified, barcode adapter-ligated target molecules are not fragmented. In some embodiments, the subset is physically separated from the fragmented population, and this separated fraction is not subjected to fragmentation. In some embodiments, fragmentation of the population is incomplete, and those molecules that escape fragmentation are used for barcode linking. In some embodiments, circularization of intact molecules brings the two barcode sequences ligated to that target molecule into proximity. In some embodiments, the region containing the two barcode sequences is separated from the target molecule (for example, by PCR or restriction endonuclease digestion), converted into sequencing-ready molecules by the addition of appropriate adapter sequences, and sequenced in the same sequencing run as the main library or in a separate run. In some embodiments, during the bioinformatic processing pipeline, these linked barcode sequence pairs are identified, and groups of reads tagged with each of the barcode sequences are merged into a single group for assembly.

In some embodiments, barcode sequences can be linked as follows: (a) circularizing (a small percentage of) the products of the first PCR amplification while forgoing the fragmentation (e.g., such that the barcode sequences at each end are brought into proximity with one another); (b) digesting the circularized molecules (e.g., with a restriction endonuclease that recognizes a specific site in the constant regions of the barcode adapter (in a some embodiments, the restriction endonuclease SapI recognizes a site in the sequence of the Illumina TruSeq™ adapter sequences)); (c) ligating asymmetric adapters to the newly exposed sticky end or ends; (d) amplifying the adapter-ligated fragments; (e) sequencing the amplified DNA (e.g., on a massively parallel short-read instrument); (f) identifying the two barcode sequences in the sequencing data; and (g) merging the two groups of reads in the primary sequencing data set defined by each of the linked barcodes. In some embodiments, the amplifying is by PCR using two oligonucleotide primers, the first of which is complementary to a constant sequence from the barcode-containing adapter, and the second of which is complementary to the overhanging sequence of the asymmetric adapter, and which together add sequences necessary for sequencing on a sequencing instrument. In some embodiments, the method further comprises assembling the two groups of reads together into longer sequences describing the target molecule that barcode adapters containing the two linked barcode sequences were ligated.

In some embodiments, as an alternative to downstream linkage of two distinct barcode sequences ligated to the two ends of the target molecule, both ends of the target molecule are tagged with the same barcode sequence. In some embodiments, a single circularization barcode adapter can be ligated to the target molecule in lieu of two end adapters. In some embodiments, the two ends of this adapter can ligate to the two ends of the same target molecule to form a circular molecule.

Without wishing to be bound by theory, it is believed that methods that attach the same barcode sequence to both ends of the target molecule via circularization, including those described herein, have advantages that include: (1) target molecules that escape barcoding can be removed by exonucleases on the basis of remaining linear; and (2) barcoded molecules can be quantified by quantitative PCR (qPCR) by amplifying a short (e.g., 50-100 bp) amplicon corresponding to sequences within the circularization adapter, rather than needing to amplify the entire target molecule.

In some embodiments, the adapter contains a single barcode sequence. In some embodiments, the barcode sequence is flanked in the 5′ direction on each strand by uracil bases. In some embodiments, after circularization, enzymes (for example, the USER™ enzyme mix (New England Biolabs)) can excise the uracils and break the phosphate backbone. In some embodiments, each strand can be broken in the 5′ direction of the barcode sequence, opening the circular molecule into a linear molecule with 5′ single-stranded overhangs at each end that contain the same barcode sequence. In some embodiments, enzymatic extension of the 3′ ends (for example, by Klenow exo-DNA polymerase or Taq DNA polymerase) copies the barcode sequence at each end, creating a fully double-stranded DNA molecule with the same barcode sequence at both ends. In some embodiments, extension by appropriate DNA polymerase enzymes leaves dA-tails useful for ligating additional adapters containing sequences that serve as PCR primer annealing sites for subsequent PCR amplification.

In some embodiments, the circularization adapter is prepared prior to ligation such that it contains two copies of the barcode sequence, or one copy of the barcode sequence and another copy of the reverse complement of that barcode sequence. In some embodiments, following circularization, the adapter is cut between the two barcodes prior to amplification. In some embodiments, it can be advantageous to circularize the target around the barcode adapter such that the same barcode sequence becomes associated with both ends of the target molecule.

In some embodiments, adapters are attached by ligation. In some embodiments, ligation is facilitated by single-nucleotide tailing. In some embodiments, the adapters are dA-tailed and the targets are dT-tailed. In some embodiments, the adapters are dT-tailed and the targets are dA-tailed. In some embodiments, adapters are attached by blunt-end ligation. In some embodiments, adapters are incorporated during amplification. In some embodiments, adapter sequences are contained within PCR primers.

In some embodiments, interior regions of the amplified target molecule are exposed prior to circularization by fragmentation. In some embodiments, fragmentation is performed using the dsDNA fragmentase enzyme mixture from New England Biolabs™, a mixture of two enzymes that creates random breaks in double-stranded DNA. Unlike exonucleases, fragmentase preserves both ends of the DNA molecule, both of which can give rise to productive circular molecules; unlike mechanical shearing, breaks are introduced along the length of the DNA molecule independent of the distance from an end or the size of the molecule; and the number of breaks per kilobase can be adjusted for different target molecule lengths by diluting the enzyme mixture or adjusting the reaction time. In some embodiments, fragmentation is achieved by mechanical shearing, or concatemerization by ligation followed by shearing.

In some embodiments, in place of mechanical or enzymatic fragmentation, fragments with random ends are generated during amplification with random (degenerate) or partially random oligonucleotide primers. In some embodiments, amplification is followed by further amplification with non-random primers. In some embodiments, amplification is followed by restriction digestion or other enzymatic treatments. In some embodiments, fragments with random ends are generated as described below (see Example 8).

In some embodiments, barcode adapter-ligated target molecules are amplified with PCR. In some embodiments, the PfuCx Turbo DNA polymerase (Agilent) is used for PCR. In some embodiments, this enzyme is compatible with uracil-containing primers, yet features a proofreading activity that reduces the error rate relative to Taq polymerases. In some embodiments, a single primer is used for PCR. It is contemplated herein that using a single primer discourages the accumulation of primer dimers during PCR (see, for example, Brown et al., Nucleic Acids Research, 1997, 26(16):3235-3241). In some embodiments, two or more distinct primers are used for PCR.

In some embodiments, the PCR mixture is supplemented with betaine, DMSO, or other additives or combinations thereof to reduce the sequence dependence of amplification efficiency, promoting a more even distribution of amplified products.

In some embodiments, the adapters that are attached to the two ends of a target molecule are identical. In some embodiments, the adapters that are attached to the two ends of a target molecule are distinct. In some embodiments, the adapters incorporate distinct PCR primer-annealing sequences and/or distinct sequencing primer-annealing sequences into the two ends of the target molecule. In some embodiments this is accomplished by adding a mixture of different adapters into the ligation mixture. In some embodiments a “forked” or “Y” adapter is used, comprising two oligonucleotides that are only partially complimentary, such that they anneal to form an adapter that is double stranded and ligation competent at one end, but forks into two non-complimentary single strands at the other end.

In some embodiments, amplification bias is reduced by a linear amplification stage prior to exponential amplification. In some embodiments, barcode-containing adapters with 3′ overhangs are attached to the ends of the target molecule, such that only one of the two strands of the ligated target molecule is capable of annealing to a PCR primer at a set annealing temperature. In some embodiments, exponential amplification is triggered by the addition of a nested primer. In some embodiments, exponential amplification is triggered by a change in the annealing temperature.

In some embodiments, amplification is achieved by rolling-circle amplification (RCA) or hyperbranching rolling-circle amplification (HRCA). In some embodiments, a circularization adapter is ligated to the target, such that the two ends of the adapter ligate to the ends of the same target molecule to form a circular molecule. In some embodiments, the adapter contains a single barcode sequence, which is flanked in the 5′ direction on each strand by nicking endonuclease recognition sequences. In some embodiments, the double-stranded DNA concatemers that result from RCA or HRCA are broken, by, for example, mechanical shearing or dsDNA fragmentase. In some embodiments, the resulting fragments are further treated with the nicking endonuclease, which introduce single stranded breaks on each side of the barcode, so that each strand of the barcode section becomes a 5′ overhang at the end of the resulting fragments. In some embodiments, Klenow or another polymerase fills in these ends, copying the barcode to create a blunt end ready for circularization. In some embodiments, two loop adapters are ligated to the ends of the target to create a circular “dumbbell” structure that can be amplified by RCA or HRCA. In some embodiments, the resulting concatemers are fragmented and digested by a nicking endonuclease as described herein.

In some embodiments, some or all of the amplification is performed within emulsified compartments.

In some embodiments, fragmented PCR products are circularized by blunt-end ligation. In some embodiments, fragmented molecules are circularized with a bridging oligonucleotide or adapter, the creation of complementary sticky ends at the ends of the fragment, or the use of recombinases.

In some embodiments, short defined sequences are designed to follow the barcode sequence in the sequencing reads to positively distinguish true barcode sequences from spurious sequences. In some embodiments, these constant sequences are selected to promote incorporation of biotinylated deoxyribonucleotides (e.g., biotin-dCTP) into the ends of fragmented molecules during end-repair.

In some embodiments, size selection is used to enrich the library for long fragments to compensate for the diminished circularization efficiency of long fragments. In some embodiments, length-dependent binding to SPRI beads is used for size selection. In some embodiments, agarose or polyacrylamide electrophoresis gel purification is used for size selection.

In some embodiments, complete or partial sequencing primer sequences are included adjacent to the random barcode sequence in the barcode adapter. This sequence can anneal in downstream PCR to an oligonucleotide that adds the full sequencing primer sequence. In some embodiments, sequences corresponding to standard manufacturer-supplied sequencing primer mixtures are incorporated to maintain compatibility with such standard primer mixtures. In some embodiments, custom sequences are used, with a corresponding custom sequence primer in place of the standard sequencing primer mixture. Without wishing to be bound by theory, it is believed that including the eventual sequencing primer sequence proximal to the barcode in the adapter can have at least two benefits:

(a) Because the sequencing read begins with the sequence directly downstream of the sequencing primer sequence, the barcode sequence is located at the beginning of one of the two paired-end sequencing reads. After the barcode sequence, the read continues directly into unknown region derived from the middle of the target molecule. This method can ensure that the random barcode is easily identified and can avoid wasting sequencing capacity by repeatedly sequencing the region on the upstream side of the barcode (which derives from the same end of the original target molecule).

(b) The presence of a primer sequence adjacent to the barcode sequence can provide a simple way to distinguish DNA fragments containing barcodes from fragments that do not contain barcodes. These latter fragments can arise when a copy of the amplified target molecule is broken more than once, creating two end fragments with barcode sequences and one or more middle fragments without barcodes. Sequencing these barcode-free fragments wastes sequencing capacity because they contain no barcode sequence to link them to a parent DNA molecule.

In some embodiments, following fragmentation, circularization, and shearing, an asymmetric adapter is ligated to both ends of the fragment. In some embodiments, this adapter is composed of two oligonucleotides, one of which is longer than the other. In some embodiments, the shorter oligonucleotide is complimentary to a portion of the longer oligonucleotide, and upon annealing creates a ligation-competent adapter with a 3′ dT-tail suitable for specific ligation to the dA-tailed fragment. In some embodiments, annealing creates a ligation-competent adapter with a 3′ dA-tail suitable for specific ligation to the dT-tailed fragment. In some embodiments, annealing creates a ligation-competent adapter with a blunt end suitable for ligation to a blunt-ended fragment. In some embodiments, the adapter sequence is complimentary to a PCR primer that adds the second sequencing primer sequence by overlap-extension PCR, but only the longer of the two oligonucleotides is long enough to productively anneal to this primer during PCR. As a result, each of the two strands of the fragment can have an annealing-competent sequence at exactly one end. In some embodiments, the second PCR primer in the reaction can anneal to the partial sequence adjacent to the barcode. As a result of this aspect, the desired fragment is in some cases the only exponentially amplified PCR product (e.g., which begins with a sequence complementary to at least part of the first sequencing primer, is followed by the barcode sequence and unknown sequence from the center of the target molecule and ends with a sequence complementary to at least part of the second sequencing primer).

In some embodiments, the method can be used to sequence the genome of an organism (e.g., an organism having multiple copies of each chromosome), single cell or virus haplotyping (e.g., B-cells, cancer stem cells, virus evolution), RNA sequencing (e.g., splice variants at multi-exon junctions, short sequence reads matching multiple sites in the genome), sequencing microbial populations (e.g., microbiome including pathogenicity islands), environmental microbiology including enzyme pathways like PKS or NRPS, or sequencing of 16S rRNA, e.g., the V4 region or full sequence.

### Methods for Linking Genotype to Phenotype

In some aspects, the sequencing methods are described herein are used in a method for linking genotype to phenotype. Biopolymers such as proteins and nucleic acids can fold into three-dimensional structures and perform a diverse set of functions. In nature, these molecules perform a range of valuable functions: they efficiently catalyze chemical reactions, selectively bind desired target molecules, serve as mechanical scaffolds, assemble into materials, etc. A number of methods have been developed for the adaptation of natural biomolecules to perform tasks of interest to humans. Such tasks include catalyzing industrially important reactions or binding to medically relevant targets in the body. Evolutionary methods have been extensively used to modify natural biomolecules. These techniques use largely random methods to generate collections (“libraries”) of variants, which are tested for the desired properties. Rational, computational, and intuitive methods are also used to design new molecules, modify natural molecules, or inform library creation. Methods for screening variants for desired properties generally fall into one of two classes. In the first class, a small enough number of variants is tested that each gene can be synthesized specifically, and each can be tested within a location (for example, a test tube or a microtiter plate well) that is known to contain that specific sequence. This type of experiment links information from any desired set of phenotypic assays with sequence information for each variant, but it is limited to a relatively small number of variants. In the second class, a larger number of variants are tested, but only a subset is selected for sequencing (nucleic acids are sequenced directly, while in the case of proteins the encoding nucleic acid is sequenced). The variant genes in this case are generally synthesized combinatorially, and their individual sequences are not known until they are determined by sequencing reactions. As before, this type of approach provides linked sequence-activity data for only a relatively small number of variants.

When multiple improved variants are found, it is often desirable to combine the causative mutations into a single variant, since the effects of beneficial mutations are often additive or compounding. Statistical methods are increasingly being incorporated into these approaches to help improve the search efficiency in the face of overwhelming combinatorial complexity. By sequencing a number of mutant genes and measuring the activity of the proteins they encode, the effects of individual mutations can be statistically isolated, and the best mutations can be identified more quickly. However, the need to either individually synthesize or individually sequence interesting variants drastically limits the amount of information that can be collected. Recently, “deep” sequencing has been used to simultaneously sequence thousands of mutants that survive a functional selection. This technique allows unprecedented statistical power. However, it is limited to binding proteins and enzymes with activity amenable to selections (for example, bond-forming enzymes or those whose activity can be linked to cell survival or growth). In addition, the prevalence of a mutant within the selected population is the only indication of its activity relative to other mutants.

In one aspect, the methods of the present disclosure fulfill a need for generation of large numbers of linked molecular genotype/phenotype pairs. In some embodiments, the genotype/phenotype pairs can be analyzed using statistical methods and can be optionally used to create biological molecules having superior and/or new properties.

In some aspects, the present disclosure fulfills a need for generation of large numbers of linked molecular genotype/phenotype pairs. In some embodiments, the genotype/phenotype pairs can be analyzed using statistical methods and can be optionally used to create biological molecules having superior and/or new properties.

In some embodiments, the sequences of nucleic acids are associated with positions on an array, and the phenotypes of the encoded variant molecules are determined in parallel at those positions. In some embodiments, measurements of the properties of interest of each variant are collected and linked to information allowing the identification, reproduction, or analysis of the sequence of each variant. In some embodiments, the methods can be applied to many types of biomolecular function and may provide a direct link between sequence information and one or more specific phenotypic characteristics. In some embodiments, the methods described herein produce linked sequence-phenotype data for a large number of variants.

In some embodiments, the variant molecules are proteins or peptides. In other embodiments, the variant molecules are nucleic acids, small molecules encoded by nucleic acids, proteins or peptides containing non-natural amino acids, or non-protein foldamers, such as peptoids or beta-peptides, encoded by nucleic acids.

Next-generation sequencing machines use massively parallel arrays to sequence millions of DNA molecules simultaneously. In some embodiments, the methods of the disclosure include modification of these, or similar machines to measure enzyme activity at the same array position at which is sequenced all or part of the encoding gene, or a short barcode sequence that can be connected to the full gene sequence. In some embodiments, an emulsion-based method can be used to attach an enzyme and its encoding DNA to the same microbead. In some embodiments, each enzyme can then be assayed for activity at the same position at which sequencing data that directly or indirectly identifies the genotype is collected. Statistical analysis of the millions of linked sequence/activity data points can then inform subsequent rounds of designs.

Read length limitations currently prevent more than a small stretch of sequence from being determined at once, but read lengths continue to increase, and within a few years sequencing of entire genes in a single read may be possible. For example, and without limitation, each position on an array can contain a nanopore-based sensor, which can detect enzymatic products as they pass through or occlude the pore, and also sequence the encoding DNA.

In some embodiments, alternatively, a sequence outside the coding region can be sequenced on the array. This region can be short enough to simplify and facilitate sequencing, yet long enough to serve as a unique identifier of the corresponding full-length gene sequence. Because this short barcode sequence can be determined on the array, at the same position as phenotypic data collection, in certain embodiments the barcode can serve to link the array address of a particular variant with genetic information that can be used to track the variant after it is removed from its position on the array. In some embodiments, the short barcode region can be amplified by emulsion PCR upstream to produce sufficient copies for sequencing. For example, these copies can be attached to the surface of the same microbead as the full gene and the protein product. It is contemplated herein that the small size of this amplicon can be conducive for efficient amplification in emulsion PCR. In some embodiments, the full gene can also be amplified in the same or a separate emulsion PCR as needed to increase protein expression. In some embodiments, the barcode sequence can be completely degenerate (i.e., poly-N), or the degeneracy can be constrained, to facilitate sequence determination. For example, and without limitation, the sequence can comprise positions allowed to be A or T alternating with positions allowed to be G or C, which can reduce or eliminate potential problems experienced by some sequencing methods when sequencing homopolymer runs. In some embodiments, the degenerate region can also be flanked or interspersed with partially or fully defined positions, e.g., to assist with quality control in downstream computational analysis. In some embodiments, the sequences can be less than completely degenerate (e.g., allowing only 1, 2, or 3 nucleotides at some or all positions).

Given a suitable long-read technology, in some aspects, the present disclosure includes sequencing a short barcode region on the array, collecting the variant genes off the array, amplifying and/or manipulating the DNA as needed to prepare it for long-read sequencing, and then sequencing the full-length genes with a long-read method to generate a single sequence that spans the barcode sequence and the full gene sequence. The full gene sequence can be thereby linked to the corresponding phenotypic information collected on the array by virtue of the barcode sequence, which is linked to the array position by sequencing on the array and linked to the full gene sequence by a long read.

Sequencing can be based on measuring fluorescence or pH. Fluorescence is commonly used to measure enzymatic activity, as fluorogenic substrates can be created for many enzymatic activities of interest. Described herein is use of fluorescence-based machines to measure the activity of an enzyme and collect information that directly or indirectly determines the sequence of its co-localized encoding gene. Examples of cyclic array sequencing by ligation or by pyrosequencing are known in the art and described in, for example and without limitation, Shendure, J., Porreca, G. J., Reppas, N. B., Lin, X., McCutcheon, J. P., Rosenbaum, A. M., Church, G. M. (2005). Accurate multiplex polony sequencing of an evolved bacterial genome. Science (New York, N.Y.), 309(5741), 1728-32. doi:10.1126/science.1117389, which is hereby incorporated in its entirety, and Margulies, M., Egholm, M., Altman, W. E., Attiya, S., Bader, J. S., Bemben, L. A., Rothberg, J. M. (2005). Genome sequencing in microfabricated high-density picolitre reactors. Nature, 437(7057), 376-80. doi:10.1038/nature03959, each of which is hereby incorporated in its entirety.

For example, the Ion Torrent PGM calls bases by detecting the minute change in pH caused by the protons released when DNA polymerase incorporates a new base (Rothberg, J. M., Hinz, W., Rearick, T. M., Schultz, J., Mileski, W., Davey, M., . . . Bustillo, J. (2011). An integrated semiconductor device enabling non-optical genome sequencing. Nature, 475(7356), 348-52. doi:10.1038/nature10242, which is hereby incorporated in its entirety).

However, many other reactions may also cause pH changes. As described herein, an apparatus containing chips, e.g., for array, may be used to provide massively parallel activity measurements and sequences of enzymes that catalyze any reaction involving the release or uptake of ions. Such methods of collecting coupled activity and sequence data from enzymes with a wide range of activities rapidly accelerate understanding of enzyme function and the engineering of enzymes with novel activities.

Described herein are methods to co-locate nucleic acids and their encoded proteins on an array, such that an apparatus capable of the parallel measurement of one or more signals (e.g., such as fluorescence, luminescence, temperature change, or pH change) can record both the sequence of all or part of the nucleic acid or a short barcode nucleic acid uniquely associated with the full nucleic acid, and the phenotype of the corresponding protein. In certain embodiments, the parallel measurement of one or more signals is via one or more sensors. In some cases, the one or more signals are proportional to a phenotype or relatable to a phenotype by a calibration curve. In some embodiments, sequence data and one or more types of phenotypic data may be collected in separate reactions, but they are linked by virtue of occurring at the same (or otherwise connected or related) physical locations on the array.

In some embodiments, the methods may similarly be used to collect linked genotype and phenotype information from nucleic acid aptamers, proteins containing non-canonical amino acids, small molecules encoded by nucleic acids, proteins or peptides alone using protein sequencing methods, and so on.

In some embodiments, DNA molecules are attached to any suitable solid support, e.g., microbeads. Attachment can be achieved by any suitable method known in the art, including for example and without limitation, binding of a biotin or double-biotin group attached to the DNA to streptavidin or avidin proteins attached to the surface of the microbeads. Accordingly, in certain embodiments this may result in each bead binding about one DNA molecule. In some embodiments, the beads may also be incubated with biotinylated primers for use in the following emulsion PCR. In some embodiments, the beads are then suspended in a solution (containing PCR reagents), which is emulsified into a continuous oil phase. Subsequently, all or a portion of the DNA is then amplified by emulsion PCR, and some fraction of the synthesized DNA copies are attached to the bead. Next, the emulsion is broken, and the beads are pooled and washed. Following such steps, the beads are ready for sequencing by any suitable technologies, including for example and without limitation the Ion Torrent, Roche/454, or Life Technologies APG systems. At this step, in some embodiments, the beads are incubated with biotinylated antibodies specific for a peptide tag. The beads are then washed and suspended in a solution containing the required components for cell-free protein synthesis. Such beads are again emulsified into an immiscible phase. Within the emulsion droplets, the clonal DNA is transcribed to produce mRNA, which is translated to produce the encoded variant protein. In some embodiments, the protein is fused to the peptide tag for which the bead-bound antibodies are specific, such that the produced protein becomes physically linked to the same bead to which is also linked its encoding DNA. The production of such microbead-DNA-protein complexes has been described in the literature (e.g., Stapleton J A, Swartz J R. Development of an In Vitro Compartmentalization Screen for High-Throughput Directed Evolution of [FeFe] Hydrogenases PLoS ONE. 2010; 5(12):e10554, which is hereby incorporated in its entirety.)

In some embodiments, the beads are then applied to an array and analyzed with an apparatus capable of (i) sequencing bead-bound DNA in parallel using technology such as that used in Ion Torrent, Roche/454, or Life Technologies APG systems, and (ii) delivering solutions to create the conditions for a desired protein assay, other than those used in the sequencing reaction, and measuring in parallel position-linked signals (e.g., fluorescence, luminescence, temperature change, or pH change) that correspond to the performance of each protein variant in the assay. Application of the parallel sequencing technology provides sequence information associated with each position on the array. All or part of the DNA can be sequenced, in one step or in multiple steps (e.g., each with different priming oligonucleotides). Prior or subsequent to sequencing, application of the parallel assay technology provides one or more measurements of the phenotype of the protein in one or more assays, again associated with each position on the array. In some embodiments, linked genotype-phenotype information can be generated for a large number of variants in parallel.

For example, and without limitation, fluorescent proteins, e.g., the green fluorescent protein (GFP), are widely used as in vivo markers in biological studies., GFP has been the target of much protein engineering to understand its function and to generate variants with improved properties such as stability, maturation speed, and altered spectral properties. The methods described herein may be used to rapidly gather a large amount of sequence-activity data for use in GFP engineering. In some embodiments, a library of biotinylated genes encoding GFP variants tagged with unique barcode sequences may be generated, for example, by error-prone PCR with a degenerate barcode region designed into one of the primers. In some embodiments, the genes are attached to microbeads and amplified by emulsion PCR. In some embodiments, the barcode region alone can be separately amplified by emulsion PCR, such that many copies of the barcode sequence are attached to the microbead. In some embodiments, the genes can be transcribed and translated by emulsion cell-free protein synthesis as described above. In some embodiments, the microbeads, which display clonal variant DNA and its encoded variant GFP protein, are applied to an array. In some embodiments, the barcode DNA on each bead is sequenced in parallel using known next-generation sequencing technology. In certain embodiments, following (or prior to) the sequencing stage, the GFP variant proteins attached to each bead are assayed. In one non-limiting example, the array is exposed to a light whose wavelength is controlled by one or more filters, and a machine measures the fluorescent light emitted from each position on the array that passes through a second set of one or more filters. In certain embodiments, multiple measurements may be performed sequentially, changing the input and output filters with each measurement to acquire detailed information on the fluorescence properties of each variant. In some embodiments, the temperature and chemical environment (e.g., the concentration of guanidinium hydrochloride or urea) may also be varied or titrated while measuring the fluorescent output of each variant, providing information on additional properties of the variants (e.g., stability). In a non-limiting example, if a superior GFP variant were present on the array, the linked sequence information collected in sequencing may be used to reproduce that protein for further characterization. Alternatively, the large number of linked sequence/phenotype measurements may be analyzed statistically to identify mutations or combinations thereof that are beneficial for GFP performance, and these mutations can be recombined in one or a few designed variants or in a new library for further rounds of screening. In some embodiments, a machine-learning algorithm is trained to predict the properties of a GFP variant of arbitrary sequence. The large datasets provided by the methods described herein may be useful in the engineering of new proteins and in furthering scientific understanding of how proteins, e.g., enzymes, fold and/or function.

In some embodiments, emulsion PCR is less efficient with longer DNA templates. In some embodiments, multiple sets of primers may be used in emulsion PCR, simultaneously or sequentially, to amplify shorter stretches of the DNA sequence. In certain embodiments, these short sequences lack an RNA polymerase promoter and are not transcribed in cell-free protein synthesis but are suitable for sequencing. In some embodiments, the entire gene can be represented in a set of such short amplicons, which can be sequenced sequentially on the array using different priming oligonucleotides. Such embodiments may include emulsion PCR to amplify the entire gene, if such amplification is necessary to eventually synthesize enough protein for the desired phenotypic assays.

Many other similar embodiments may be imagined by those of skill in the art. For example, in some embodiments, emulsion PCR could be omitted, or replaced with in vitro transcription, and optionally, followed by reverse-transcription. Alternatively, in some embodiments, biotinylated RNA could be transcribed in bulk solution and then attached to microbeads.

While the above descriptions have focused on the binding of molecules to microbeads, the methods are not limited in this regard. For example, in certain embodiments nucleic acids can be bound directly to surfaces such as glass. In certain embodiments, the encoded proteins can be synthesized prior to or following nucleic acid binding to the chip and bound to the same surface or to the nucleic acids themselves (e.g., by ribosome display, RNA display, or DNA display). Surface-bound nucleic acids can then optionally be amplified before or after transcription or translation by methods including bridge PCR. Binding the nucleic acids to a surface may allow other high-throughput sequencing technologies to be used, e.g., those developed by Illumina/Solexa and Helicos BioSciences. Alternatively, in some embodiments, single nucleic acid/protein complexes such as those that result from ribosome display, RNA display, or DNA display can be sequenced by technologies such as those developed by Pacific Biosciences, or by nanopore sequencing.

In some embodiments, the active molecule is RNA rather than protein. In such embodiments, a number of approaches can be used, including but not limited to the following:

(i) a protocol similar to the microbead-attachment protocol described above can be used, but the cell-free protein synthesis is replaced by in vitro transcription within the emulsion. The phenotypes of the resulting RNAs are measured as described above (e.g., pH changes).

(ii) a microbead-attachment protocol can be used, wherein the DNA and the microbead are co-compartmentalized during an in vitro transcription that results in decoration of the microbead with RNA. The RNA is then sequenced directly or reverse-transcribed to generate DNA for sequencing.

(iii) single molecules of RNA are attached to beads, surfaces, or surface-bound molecules such as polymerases, and sequenced directly or reverse-transcribed to generate DNA for sequencing, prior to or following single-molecule characterization.

In some embodiments, for example, where assessing enzymatic rates are of interest, methods are described herein for estimating approximately how many copies of the enzyme were bound to a given microbead during protein synthesis. This can be accomplished in a number of ways. For example, and without limitation, the enzyme can be linked at a defined stoichiometry to a molecule or fusion of known characteristics. Measurement of a signal from the array position specific to this calibration molecule allows determination of the number of copies of the molecule of interest at each position in the array. For example, and without limitation, the number of these control molecules can be determined by measuring change in parameters such as fluorescence, luminescence, temperature change, or pH as a result of enzymatic activity or binding to a probe molecule, e.g., a probe molecule such as an antibody linked to a fluorescent molecule, an enzyme, or an enzymatic substrate.

In some embodiments, for example, where assessing binding is of interest, the molecule to be bound is conjugated or fused to an enzyme capable of generating a signal with a high turnover rate, so that each bound molecule generates an amplified signal to facilitate detection. In some embodiments, the substrate and/or product of this reaction is attached to microbeads or to the array surface to preserve the localization of the signal within the particular array position.

In some embodiments, the nucleic acid sequences to be tested are spotted or printed directly onto known positions on the array. This can be done by any one of a number of suitable technologies as known in the art, including but not limited to inkjet or photolithography-based methods. In some embodiments, the nucleic acid is RNA. In some embodiments, the nucleic acid is DNA, in which case it may be transcribed by any suitable method that preserves the spatial information that locates the nucleic acid sequence on the array. An exemplary suitable method is ligation between the DNA and corresponding RNA. In some embodiments, array-bound RNA may be translated using methods such as ribosome display or RNA display, wherein the newly synthesized protein remains spatially associated with its encoding RNA or DNA or the array. Alternatively, in some embodiments, peptides or proteins with specific sequences can be synthesized directly onto defined positions on the array by solid-phase synthesis. In these embodiments, sequencing is not necessary, as the sequence of the nucleic acid printed in each location is known. Phenotypic characterization then takes place in parallel on the array as described.

In some embodiments, oligonucleotides containing “barcode” sequences, each of which refer to a specific full-length variant gene, are printed onto an array. In some embodiments, nucleic acid/protein complexes then attach to the array by way of hybridization between the nucleic acid and the bound oligonucleotides. In some embodiments, the nucleic acids contain complementary barcode sequences that allow specific annealing to a particular array-bound oligonucleotide. In some embodiments, nucleic acid/protein complexes (where the nucleic acid can be RNA or DNA, and can be complexed with its encoded protein by ribosome display, RNA display, DNA display, mutual attachment to a microbead, and so on) are synthesized and assembled in bulk solution and then directed to known positions on an array. In such embodiments, on-array sequencing is therefore not needed, and long-read sequencing can be subsequently performed if necessary to link the barcode sequences with the full-length gene sequences. Parallel, location-linked phenotypic characterization then takes place as described herein. The protein-associated nucleic acid could contain the open reading frame along with the barcode, or it could contain only the barcode. The latter scenario could be accomplished by, for example and without limitation, binding a nucleic acid molecule comprising a barcode and an open reading frame to a microbead, and amplifying only the barcode section by emulsion PCR such that the bead becomes decorated with many copies of the barcode sequence. Alternatively, a method similar to DNA display could be used to attach a barcode sequence directly to the protein.

The methods of the disclosure can also be applied in many other areas of science and engineering. For example, it could be used to rapidly characterize unknown open reading frames from, e.g., environmental samples. These genes could be expressed, displayed on the array, and exposed sequentially to a battery of tests, e.g., for common enzymatic activities, binding partners, biophysical properties, and the like.

In some aspects, the method may be used to modify the properties of an existing enzyme or ribozyme by directed evolution. Accordingly, in some embodiments, a mutant library is generated from a starting parent gene. The library is then analyzed using the described method, which provides data describing the complete or partial sequence and phenotype of each mutant. This data is then used to generate a new mutant library, which can be based on one or more mutants with desirable properties identified by the method. Alternatively, the library can be combinatorially assembled from oligonucleotides containing one or more mutations identified by the method as being statistically associated with desirable phenotypes. Optionally, this process is iteratively repeated for as many cycles as desired.

In certain embodiments, it may be desirable to sequence the nucleic acids more than once while maintaining their positions on the array, for example, to ensure sequencing accuracy. Many parallel sequencing technologies have read lengths that are short relative to the length of a typical gene. In some embodiments, different regions of a nucleic acid may be sequenced in multiple sequential sequencing runs. These partial sequences may then be collected sequentially but remain associated with the same array position. The partial sequences may then be combined using overlapping regions or by comparison to a known parent or reference sequence. The partial sequences may be generated by sequencing regions of the same nucleic acid molecule. Alternatively, sections of the long nucleic acid polymer that contains the open reading frame can be individually amplified to create a number of smaller nucleic acid molecules, which remain associated with the parent molecule, e.g., by binding to the same bead following emulsion PCR. These smaller nucleic acids can then be sequenced, and these partial sequences combined as described previously.

In some aspects, an array described herein comprises at least about 1, 2, 10¹, 10², 10¹, 10⁴, 10¹, 10⁶, 10⁷, 10⁸, 10⁹, 10¹⁰, 10¹¹ or more sensors. In some aspects, an array described herein comprises at most about 10¹¹, 10¹⁰, 10⁹, 10⁸, 10⁷, 10⁶, 10⁵, 10⁴, 10³, 10², 10¹, 2 sensors, or 1 sensor. A sensor may measure a signal associated with a signal associated with fluorescence, pH change, temperature change, luminescence, or any combination thereof. In some aspects, an array described herein may be interrogated by a sensor. Such a sensor may measure a signal associated with a signal associated with fluorescence, pH change, luminescence, temperature change or any combination thereof associated with the array. In some aspects, an array comprises one or more chemical field-effect transistor (chemFET) sensors.

In some aspects, a phenotype described herein may be any phenotype of interest. Non-limiting examples of phenotypes include enzyme specificity, binding affinity, binding specificity and stability when exposed to a chemical condition or a temperature. In some aspects, a method includes contacting proteins to a plurality of solutions comprising substrates at a plurality of concentrations. In some aspects, a method includes contacting proteins to a plurality of solutions comprising ligands at a plurality of concentrations. In some aspects, a method includes measuring a phenotype at a plurality of temperatures.

### Computer Control Systems

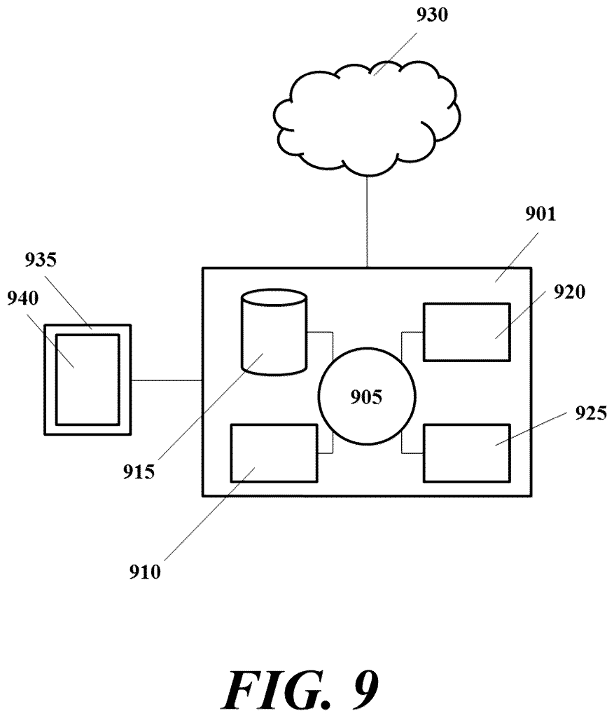

In some embodiments, the present disclosure provides computer control systems that are programmed to implement methods of the disclosure. For example, FIG. 9 shows a computer system 901 that is programmed or otherwise configured to operate instrumentation (e.g., a thermal cycler, fluid handling apparatuses including pumps and valves, a sequencing instrument, a sequencing platform, etc.), analyze and store sequencing reads, perform sequence assembly, store results of a sequence assembly, and/or display data (e.g., results of sequencing analysis, instrument operational parameters, etc.). The computer system 901 can regulate various aspects of devices (e.g., thermal cyclers, fluid handling apparatuses including pumps and valves, sequencing instrumentation, sequencing platforms, etc.), sequence read analysis methods, and sequence assembly methods described herein. The computer system 901 can be an electronic device of a user or a computer system that is remotely located with respect to the electronic device. The electronic device can be a mobile electronic device.

In some embodiments, the computer system 901 includes a central processing unit (CPU, also referred to as “processor” and “computer processor” herein) 905, which can be a single core or multi core processor, or a plurality of processors for parallel processing. In certain embodiments, the computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. In such embodiments, the memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930, in some cases, is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905, without limitation, can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901, in some cases, can include one or more additional data storage units that are external to the computer system 901, such as located on a remote server that is in communication with the computer system 901 through an intranet or the Internet.

The computer system 901 can communicate with one or more remote computer systems through the network 930. For instance, it may be that the computer system 901 can communicate with a remote computer system of a user. Examples of remote computer systems include, without limitation, personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium, or physical transmission medium. Non-volatile storage media include, for example and without limitation, optical or magnetic disks, such as any of the storage devices in any computer(s) or the like, such as may be used to implement the databases, etc. shown in FIG. 9. Volatile storage media may include, for example and without limitation, dynamic memory, such as main memory of such a computer platform. Tangible transmission media may include, for example and without limitation, coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore may include, for example and without limitation: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

In some aspects, the computer system 901 can include or otherwise be in communication with an electronic display 935 that comprises a user interface (UI) 940 for providing, for example, operation parameters of an instrument. Such operation parameters may include, for example, a thermal cycler, a sequencing instrument, fluid handling instrumentation; alternatively, the UI may include instrument performance, parameters of a sequence assembly method, results, associated statistics of a sequence assembly data, etc. Examples of suitable UIs are known in the art and include, without limitation, a graphical user interface (GUI) and web-based user interface.

In some aspects, methods and systems of the present disclosure can be implemented by way of one or more algorithms. When desired, an algorithm can be implemented by way of software upon execution by the central processing unit 905.

The algorithm can, for example, initiate electronic signals that are processed to operate instrumentation (e.g., a thermal cycler, fluid handling apparatuses (including but not limited to pumps and valves), a sequencing instrument, a sequencing platform, etc.), analyze and store sequencing reads, perform sequence assembly and/or store results, display data (e.g., results of sequencing analysis, instrument operational parameters, etc.) to a user, transmit to or receive data from a remote computer system, etc.

### Single-Stranded Splint Workflow

### Methods for Forming a Plurality of Library-Splint Complexes

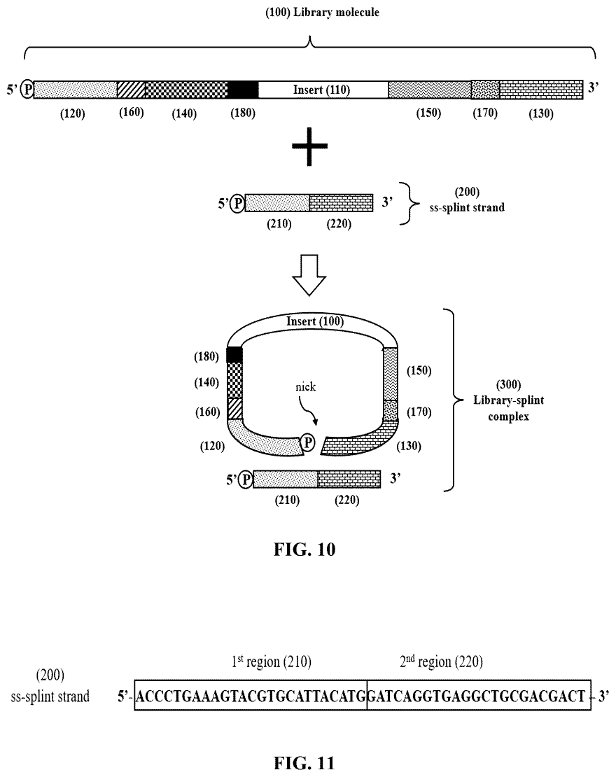

In some aspects, the present disclosure provides methods for forming a plurality of library-splint complexes (300) comprising: step (a) providing a plurality of single-stranded nucleic acid library molecules (100) wherein individual library molecules in the plurality comprise regions arranged in a 5′ to 3′ order: (i) a surface pinning primer binding site (120), (ii) a left sample index sequence (160), (iii) a forward sequencing primer binding site (140), (iv) a left UMI sequence (180), (v) an insert sequence (e.g., sequence of interest) (110), (vi) a reverse sequencing primer binding site (150), (vii) a right sample index sequence (170) which optionally includes a 3-mer random sequence, and (viii) a surface capture primer binding site (130). An exemplary library molecule is shown in FIG. 10. In some embodiments, the length of the insert sequence is about 25-1000 nucleotides, or about 1000-20,000 nucleotides, or about 20,000-500,000 nucleotides. In some embodiments, the library molecules include one UMI sequence, for example a left UMI sequence (180) or a right UMI sequence (190). In some embodiments, the right UMI sequence (190) is located between the insert sequence (110) and the reverse sequencing primer binding site (150). In some embodiments, the library molecules include two UMI sequences, for example a left (180) and right UMI (190) sequence. In some embodiments, the left sample index sequence (160) can be 3-20 nucleotides in length. In some embodiments, the right index sequence (170) can be 3-20 nucleotides in length.

In some embodiments, the left sample index sequence (160) and/or the right sample index sequence (170) can include a short random sequence (e.g., NNN) which can be 3-20 nucleotides in length. The sequences of the left and right sample index sequences (e.g., (160) and (170)) can be the same. Alternatively, the sequences of the left and right sample index sequences (e.g., (160) and (170)) can be different from each other. The sample index sequences can be used to distinguish sequences of interest obtained from different sample sources in a multiplex assay.

In some aspects, multiplex workflows are enabled by preparing sample-indexed libraries using one or both index sequences (e.g., one or both of the left and/or right index sequences). The first left index sequences (160) and/or first right index sequences (170) can be employed to prepare separate sample-indexed libraries using input nucleic acids isolated from different sources. The sample-indexed libraries can be pooled together to generate a multiplex library mixture, and the pooled libraries can be circularized, amplified, and/or sequenced. Accordingly, the sequences of the insert region along with the first left index sequence (160) and/or first right index sequence (170) can be used to identify the source of the input nucleic acids. In some embodiments, any number of sample-indexed libraries can be pooled together, for example 2-10, 10-50, 50-100, 100-200, or more than 200 (e.g., about 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, or more than 200) sample-indexed libraries can be pooled. Exemplary nucleic acid sources include, without limitation, naturally occurring, recombinant, or chemically synthesized sources. Exemplary nucleic acid sources include, without limitation, single cells, a plurality of cells, tissue, biological fluid, an environmental sample, or a whole organism. Exemplary nucleic acid sources include, without limitation, fresh, frozen, fresh-frozen or archived sources (e.g., formalin-fixed paraffin-embedded; FFPE). The skilled artisan will recognize that the nucleic acids can be isolated from many other sources. The nucleic acid library molecules can be prepared in single-stranded or double-stranded form.

In some embodiments, the left UMI (180) comprises a unique molecular index and/or the right UMI (190) comprises a unique molecular index that are used to uniquely identify an individual sequence of interest (e.g., insert sequence) to which the UMI is/are appended in a population of other sequence of interest molecules. In some embodiments, the left UMI (180) and/or the right UMI (190) can be used for molecular tagging. In some embodiments, the left UMI (180) and/or right UMI (190) comprise 2-20 (e.g., about 2, 3, 4, 5, 6, 7, 8, 9, 10, 11, 12, 13, 14, 15, 16, 17, 18, 19, or 20) or more nucleotides having a known sequence. For example, in some embodiments, the left UMI (180) and/or right UMI (190) comprise a known random sequence where a nucleotide at each position is randomly selected from nucleotides having a base A, G, C, T, or U. The left UMI (180) and/or right UMI (190) can be used for molecular tagging procedures. An example embodiment of a single-stranded nucleic acid library molecule having a left UMI (180) is shown in FIG. 10.

In some embodiments, in the methods for forming a plurality of library-splint complexes, the surface pinning primer binding site (120) in the library molecules comprise the sequence

(SEQ ID NO: 20)
5′-CATGTAATGCACGTACTTTCAGGGT-3′.

In some embodiments, in the methods for forming a plurality of library-splint complexes, the forward sequencing primer binding site (140) in the library molecules comprise the sequence

(SEQ ID NO: 22)
5′-CGTGCTGGATTGGCTCACCAGACACCTTCCGACAT-3′.

In some embodiments, in the methods for forming a plurality of library-splint complexes, the reverse sequencing primer binding site (150) in the library molecules comprise the sequence

(SEQ ID NO: 23)
5′-ATGTCGGAAGGTGTGCAGGCTACCGCTTGTCAACT-3′.

In some embodiments, in the methods for forming a plurality of library-splint complexes, the surface capture primer binding site (130) in the library molecules comprise the sequence

(SEQ ID NO: 24)
5′-AGTCGTCGCAGCCTCACCTGATC-3′.

In some embodiments, the methods for forming a plurality of library-splint complexes (300) further comprises step (b): providing a plurality of single-stranded splint strands (200) wherein individual single-stranded splint strands (200) comprises regions arranged in a 5′ to 3′ order (i) a first region (210) having a universal binding sequence that hybridizes with a sequence on one end of the linear single stranded library molecule, for example the surface pinning primer binding site (120); and (ii) a second region (220) having a universal binding sequence that hybridizes with a sequence on the other end of the linear single stranded library molecule, for example, the surface capture primer binding site (130). An example embodiment of a single-stranded splint strand (200) is shown in FIG. 11.

In some embodiments, methods for forming a plurality of library-splint complexes, the first region of the single-stranded splint strand (210) includes a universal binding sequence for a first left universal adaptor sequence (120) of a library molecule, where the first region (210) comprises the sequence 5′-ACCCTGAAAGTACGTGCATTACATG-3′ (SEQ ID NO:25) (e.g., FIG. 11).

In some embodiments, methods for forming a plurality of library-splint complexes, the second region of the single-stranded splint strand (220) includes a universal binding sequence for a first right universal adaptor sequence (130) of a library molecule, where the second region (220) comprises the sequence 5′-GATCAGGTGAGGCTGCGACGACT-3′ (SEQ ID NO:26) (e.g., FIG. 11).

In some embodiments, methods for forming a plurality of library-splint complexes, the single-stranded splint strand (200) comprises the sequence 5′-ACCCTGAAAGTACGTGCATTACATGGATCAGGTGAGGCTGCGACGACT-3′ (SEQ ID NO:27). For a non-limiting example, see FIG. 11.

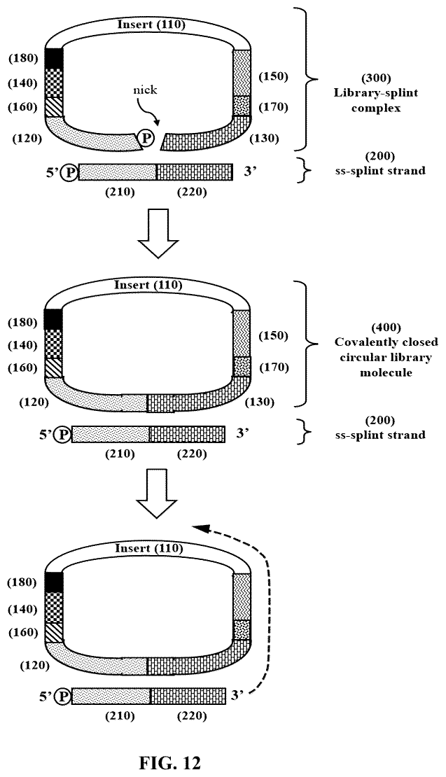

In some embodiments, the methods for forming a plurality of library-splint complexes (300) further comprises step (c): forming a library-splint complex (300) by hybridizing the plurality of single-stranded nucleic acid library molecules (100) with the plurality of single-stranded splint strands (200) under a condition suitable to hybridize the first region (210) of the single-stranded splint strand to the surface pinning primer binding site (120) of the single-stranded library molecule, and under a condition suitable to hybridize the second region (220) of the single-stranded splint strand to the surface capture primer binding site (130) of the single-stranded library molecule, wherein the library-splint complex (300) comprises a nick between the terminal 5′ and 3′ ends of the library molecule, and wherein the nick is enzymatically ligatable (e.g., see FIGS. 10 and 12).

In some embodiments, the methods for forming a plurality of library-splint complexes (300) further comprises step (d): contacting the library-splint complexes (300) with a plurality of ligase enzymes under a condition suitable to enzymatically ligate the nick, thereby generating a plurality of covalently closed circular library molecules (400), each hybridized to a single-stranded splint strand (200) (e.g., FIGS. 10 and 12). In some embodiments, the ligase enzyme comprises T7 DNA ligase, T3 ligase, T4 ligase, or Taq ligase.

In some embodiments, the methods for forming a plurality of library-splint complexes (300) further comprises optional step (d): enzymatically removing the plurality of single-stranded splint strands (200) from the plurality of covalently closed circular library molecules (400) by contacting the plurality of single-stranded splint strands (200) with at least one exonuclease enzyme to remove the plurality of single-stranded splint strands (200) and retaining the plurality of covalently closed circular library molecules (400). In some embodiments, the at least one exonuclease enzyme comprises any combination of one or more of exonuclease I, thermolabile exonuclease I, and/or T7 exonuclease.

In some embodiments, the plurality of single-stranded splint strands (200) is retained (e.g., they are not removed or degraded). In such embodiments, the single-stranded splint strands (200) can be used as primers, e.g., to initiate a rolling circle amplification reaction using the covalently closed circular library molecules (400) as template molecules to generate concatemer molecules. For a non-limiting example, see FIG. 12.

### Double-Stranded Splint Workflow

### Methods for Forming a Plurality of Library-Splint Complexes

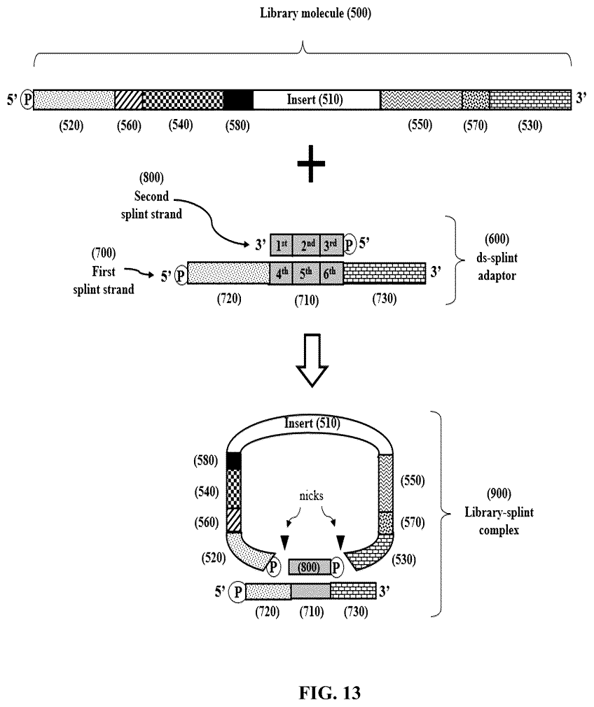

In some aspects, the present disclosure provides methods for forming a plurality of library-splint complexes (900) comprising: step (a) providing a plurality of single-stranded nucleic acid library molecules (500) wherein individual library molecules in the plurality comprise regions arranged in a 5′ to 3′ order: (i) a surface pinning primer binding site (520), (ii) a left sample index sequence (560), (iii) a forward sequencing primer binding site (540), (iv) a left UMI sequence (580), (v) an insert sequence (e.g., sequence of interest) (510), (vi) a reverse sequencing primer binding site (550), (vii) a right sample index sequence (570) which optionally includes a 3-mer random sequence, and (viii) a surface capture primer binding site (530). An exemplary library molecule is shown in FIG. 13. In some embodiments, the length of the insert sequence is about 25-1,000 nucleotides, about 1,000-20,000 nucleotides, or about 20,000-500,000 nucleotides. In some embodiments, the library molecules include one UMI sequence, for example a left UMI sequence (580) or a right UMI sequence (590). In some embodiments, the right UMI sequence (590) is located between the insert sequence (510) and the reverse sequencing primer binding site (550). In some embodiments, the library molecules include two UMI sequences, for example a left (580) and right UMI (590) sequence. In some embodiments, the left sample index sequence (560) can be 3-20 nucleotides (e.g., 3, 4, 5, 6, 7, 8, 9, 10, 11, 12, 13, 14, 15, 16, 17, 18, 19, or 20 nucleotides) in length. In some embodiments, the right index sequence (570) can be 3-20 nucleotides (e.g., 3, 4, 5, 6, 7, 8, 9, 10, 11, 12, 13, 14, 15, 16, 17, 18, 19, or 20 nucleotides) in length.

In some embodiments, the left sample index sequence (560) and/or the right sample index sequence (570) can include or lack a short random sequence (e.g., NNN) which can be 3-20 nucleotides (e.g., 3, 4, 5, 6, 7, 8, 9, 10, 11, 12, 13, 14, 15, 16, 17, 18, 19, or 20 nucleotides) in length. The sequences of the left and right sample index sequences (e.g., (560) and (570)) can be the same or different from each other. The sample index sequences can be used to distinguish sequences of interest obtained from different sample sources in a multiplex assay.

Multiplex workflows are enabled by preparing sample-indexed libraries using one or both index sequences (e.g., left and/or right index sequences). The first left index sequences (560) and/or first right index sequences (570) can be employed to prepare separate sample-indexed libraries using input nucleic acids isolated from different sources. The sample-indexed libraries can be pooled together to generate a multiplex library mixture, and the pooled libraries can then be circularized, amplified and/or sequenced. The sequences of the insert region along with the first left index sequence (560) and/or first right index sequence (570) can be used to identify the source of the input nucleic acids. In some embodiments, any number of sample-indexed libraries can be pooled together, for example, 2-10, 10-50, 50-100, 100-200, or more than 200 (e.g., about 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, or more than 200) sample-indexed libraries can be pooled. Exemplary nucleic acid sources include, without limitation, naturally occurring, recombinant, or chemically-synthesized sources. Exemplary nucleic acid sources include, without limitation, single cells, a plurality of cells, tissue, biological fluid, an environmental sample, or a whole organism. Exemplary nucleic acid sources include, without limitation, fresh, frozen, fresh-frozen or archived sources (e.g., formalin-fixed paraffin-embedded; FFPE). The skilled artisan will recognize that the nucleic acids can be isolated from many other sources. The nucleic acid library molecules can be prepared in single-stranded or double-stranded form.

In some embodiments, the left UMI (580) comprises a unique molecular index and/or the right UMI (590) comprises a unique molecular index, such UMI can be used to uniquely identify an individual sequence of interest (e.g., insert sequence) to which the UMI is/are appended in a population of other sequence of interest molecules. In some embodiments, the left UMI (580) and/or the right UMI (590) can be used for molecular tagging. In some embodiments, the left UMI (580) and/or right UMI (590) comprise 2-20 or more nucleotides (e.g., 2, 3, 4, 5, 6, 7, 8, 9, 10, 11, 12, 13, 14, 15, 16, 17, 18, 19, or 20 nucleotides) having a known sequence. For example, the left UMI (580) and/or right UMI (590) may comprise a known random sequence where a nucleotide at each position is randomly selected from nucleotides having a base A, G, C, T or U. The left UMI (580) and/or right UMI (590) can be used for molecular tagging procedures. An exemplary embodiment of a single-stranded nucleic acid library molecule having a left UMI (580) is shown in FIG. 13.

In some embodiments, in the methods for forming a plurality of library-splint complexes, the surface pinning primer binding site (520) in the library molecules comprise the sequence 5′-AATGATACGGCGACCACCGA-3′ (SEQ ID NO:30).

In some embodiments, in the methods for forming a plurality of library-splint complexes, the forward sequencing primer binding site (540) in the library molecules comprise the sequence 5′-ACACTCTTTCCCTACACGACGCTCTTCCGATCT-3′ (SEQ ID NO:31).

In some embodiments, in the methods for forming a plurality of library-splint complexes, the forward sequencing primer binding site (540) in the library molecules comprise the sequence 5′-TCGTCGGCAGCGTCAGATGTGTATAAGAGACAG-3′ (SEQ ID NO:32).

In some embodiments, in the methods for forming a plurality of library-splint complexes, the reverse sequencing primer binding site (550) in the library molecules comprise the sequence 5′-AGATCGGAAGAGCACACGTCTGAACTCCAGTCAC-3′ (SEQ ID NO:33).

In some embodiments, in the methods for forming a plurality of library-splint complexes, the surface capture primer binding site (530) in the library molecules comprise the sequence 5′-CTGTCTCTTATACACATCTCCGAGCCCACGAGAC-3′ (SEQ ID NO:34).

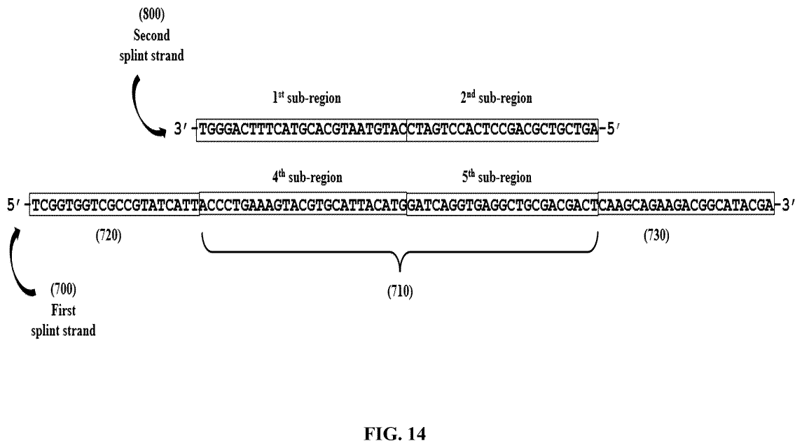

In some embodiments, the methods for forming a plurality of library-splint complexes (900) further comprises step (b): providing a plurality of double-stranded splint adaptors (600) wherein individual double-stranded splint adaptors (600) comprises a first splint strand (e.g., a long splint strand) (700) and a second splint strand (e.g., a short splint strand) (800). In some embodiments, individual double-stranded splint adaptors (600) in the plurality comprise a first splint strand (700) hybridized to a second splint strand (800). Exemplary embodiments of a double-stranded splint adaptor (600) are shown in FIGS. 13 and 14.

In some embodiments, the first splint strand (700) comprises regions arranged in a 5′ to 3′ order (i) a first region (720); (ii) an internal region (710); and (iii) a second region (730) (FIG. 13). In some embodiments, the first region (720) of the first splint strand (700) comprises a sequence that hybridizes with the surface pinning primer binding site (520) in the library molecules (500). In some embodiments, the second region (730) of the first splint strand (700) comprises a sequence that hybridizes with the surface capture primer binding site (530) in the library molecules (500). In some embodiments, the internal region (710) of the first splint strand (700) comprises a fourth, fifth and an optional sixth sub-region. In some embodiments, the fourth sub-region comprises a sequence (or a complementary sequence thereof) that can hybridize with an SP5 surface pinning primer. In some embodiments, the fifth sub-region comprises a sequence (or a complementary sequence thereof) that can hybridize with an SP27 surface pinning primer. In some embodiments, the optional sixth sub-region comprises a unique molecular index (UMI) that can be used to uniquely identify an individual sequence of interest (e.g., insert sequence) to which the UMI is/are appended in a population of other sequence of interest molecules.

In some embodiments, the second splint strand (800) comprises regions arranged in a 5′ to 3′ order (i) a third sub-region (720); (ii) a second sub-region (710); and (iii) a first sub-region (FIG. 13). In some embodiments, the third sub-region of the second splint strand (800) hybridizes to the sixth region of the first splint strand (700). In some embodiments, the second sub-region of the second splint strand (800) hybridizes to the fifth region of the first splint strand (700). In some embodiments, the first sub-region of the second splint strand (800) hybridizes to the fourth region of the first splint strand (700). In some embodiments, the fourth and fifth sub-regions of the first splint strands (700) do not hybridize (or at least exhibit very little hybridization to) the SP27 surface capture primers or the SP5 surface pinning primers.

In some embodiments, in the methods for forming a plurality of library-splint complexes described herein, the first region (720) of the first splint strand (700) comprises a short P5 sequence

(FIG. 14)
(SEQ ID NO: 36)
5′-TCGGTGGTCGCCGTATCATT-3′.

In some embodiments, in the methods for forming a plurality of library-splint complexes, the first region (720) of the first splint strand (700) comprises a long P5 sequence

(SEQ ID NO: 37)
5′-AATGATACGGCGACCACCGAGATC-3′.

In some embodiments, in the methods for forming a plurality of library-splint complexes, the second region (730) of the first splint strand (700) comprises a short P7 sequence

(FIG. 14)
(SEQ ID NO: 38)
5′-CAAGCAGAAGACGGCATACGA-3′.

In some embodiments, in the methods for forming a plurality of library-splint complexes, the second region (730) of the first splint strand (700) comprises a long P7 sequence

(SEQ ID NO: 39)
5′-CAAGCAGAAGACGGCATACGAGAT-3′.

In some embodiments, in the methods for forming a plurality of library-splint complexes, the fourth sub-region of the first splint strand (700) comprises an SP5′ sequence

(FIG. 14)
(SEQ ID NO: 40)
5′-ACCCTGAAAGTACGTGCATTACATG-3′.

In some embodiments, in the methods for forming a plurality of library-splint complexes, the fifth sub-region of the first splint strand (700) comprises an SP27 sequence

(FIG. 14)
(SEQ ID NO: 41)
5′-GATCAGGTGAGGCTGCGACGACT-3′.

In some embodiments, in the methods for forming a plurality of library-splint complexes, the full-length sequence of the first splint strand (700) comprises

(e.g., FIG. 14)
(SEQ ID NO: 42)
TCGGTGGTCGCCGTATCATT
ACCCTGAAAGTACGTGCATTACATGGATCAGGTGAGGCTGCGACGACTC
AAGCAGAAGACGGCATACGA-3′.

In some embodiments, in the methods for forming a plurality of library-splint complexes, the first sub-region of the second splint strand (800) comprises the sequence

(FIG. 14)
(SEQ ID NO: 43)
5′-CATGTAATGCACGTACTTTCAGGGT-3′.

In some embodiments, in the methods for forming a plurality of library-splint complexes, the second sub-region of the second splint strand (800) comprises the sequence

(FIG. 14)
(SEQ ID NO: 44)
5′-AGTCGTCGCAGCCTCACCTGATC-3′.

In some embodiments, in the methods for forming a plurality of library-splint complexes, the full-length sequence of the second splint strand (800) comprises

(e.g., FIG. 14)
(SEQ ID NO: 45)
5′-AGTCGTCGCAGCCTCACCTGATCCATGTAATGCACGTACTTTCAGG
GT-3′.

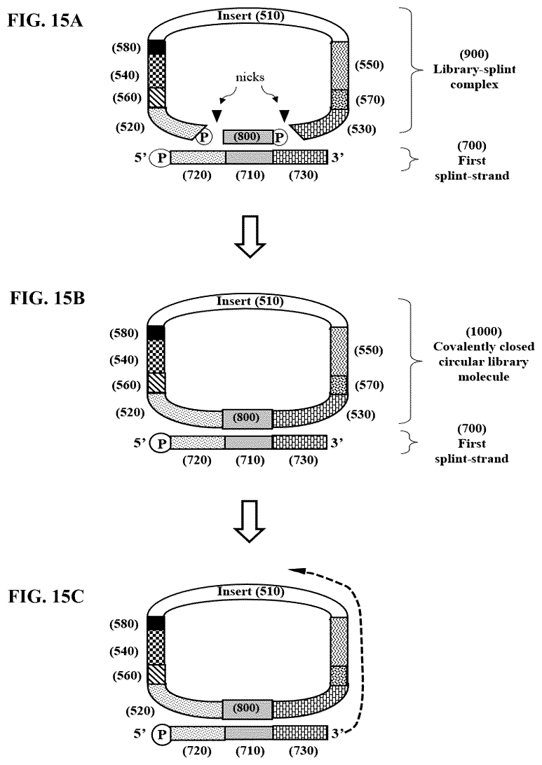

In some embodiments, the methods for forming a plurality of library-splint complexes (900) further comprises step (c): forming a library-splint complex (900) by hybridizing the plurality of single-stranded nucleic acid library molecules (500) with the plurality of double-stranded splint strands (600) under a condition suitable to hybridize the first region (720) of the first splint strand to the surface pinning primer binding site (520) of the single-stranded library molecule (500), and under a condition suitable to hybridize the second region (730) of the first splint strand (700) to the surface capture primer binding site (530) of the single-stranded library molecule (500), wherein the library-splint complex (900) comprises a first nick between the 5′ end of the library molecule and the 3′ end of the second splint strand (800). In certain embodiments, the library-splint complex (900) also comprises a second nick between the 5′ end of the second splint strand (800) and the 3′ end of the library molecule (e.g., FIG. 15). In some embodiments, the first and second nicks are enzymatically ligatable.

In some embodiments, the methods for forming a plurality of library-splint complexes (900) further comprises step (d): contacting the library-splint complexes (900) with a plurality of ligase enzymes under a condition suitable to enzymatically ligate the nick, thereby generating a plurality of covalently closed circular library molecules (1000), each hybridized to a first splint strand (700) (e.g., FIGS. 15A, 15B and 15C). In some embodiments, the ligase enzyme comprises T7 DNA ligase, T3 ligase, T4 ligase, or Taq ligase.

In some embodiments, the methods for forming a plurality of library-splint complexes (900) further comprises optional step (d): enzymatically removing the plurality of first splint strands (700) from the plurality of covalently closed circular library molecules (1000) by contacting the plurality of first splint strands (700) with at least one exonuclease enzyme to remove the plurality of first splint strands (700) and retaining the plurality of covalently closed circular library molecules (1000). In some embodiments, the at least one exonuclease enzyme comprises any combination of one or more of exonuclease I, thermolabile exonuclease I and/or T7 exonuclease.

In some embodiments, the plurality of first splint strands (700) are retained (e.g., they are not removed or degraded). In such embodiments, the first splint strands (700) can be used as primers to initiate a rolling circle amplification reaction using the covalently closed circular library molecules (1000) as template molecules to generate concatemer molecules. For a non-limiting example, see FIGS. 15A, 15B, and 15C.

In some embodiments, the plurality of covalently closed circular library molecules (1000) can hybridize to an amplification primer, where the amplification primer is in-solution or immobilized to a support, and the plurality of covalently closed circular library molecules (1000) can then be subjected to a rolling circle amplification reaction to generate a plurality of concatemers. In some embodiments, the amplification primers comprise the sequence 5′-GATCAGGTGAGGCTGCGACGACT-3′ (SEQ ID NO:28). In some embodiments, the amplification primers comprise immobilized capture primers having the sequence 5′-GATCAGGTGAGGCTGCGACGACT-3′ (SEQ ID NO:28). In some embodiments, at least one portion of the concatemers can hybridize to immobilized pinning primers comprising the sequence 5′-CATGTAATGCACGTACTTTCAGGGT-3′ (SEQ ID NO:29).

### On-Support Rolling Circle Amplification

In some embodiments, the plurality of covalently closed circular molecules (400) can be distributed onto a coated support and can serve as template molecules in a rolling circle amplification reaction to generate immobilized concatemer molecules. The immobilized concatemer molecules can be subjected to multiple cycles of sequencing reactions.

In some embodiments, the methods for conducting rolling circle amplification reaction on a plurality of covalently closed circular library molecules which lack hybridized single-stranded splint strands (200), and wherein individual covalently closed circular library molecules (400) in the plurality comprise a universal binding sequence for a first surface primer (e.g., surface capture primer), comprise step (a): distributing the plurality of covalently closed circular library molecules (400) onto a support having a plurality of the first surface primers immobilized on the support, under a condition suitable for hybridizing individual covalently closed circular library molecules (400) to individual immobilized first surface primers thereby immobilizing the plurality of covalently closed circular library molecules (400) to the support. In some embodiments, the rolling circle amplification reaction includes contacting the immobilized the plurality of covalently closed circular library molecules with strand displacing polymerase and a plurality of nucleotides (e.g., dATP, dCTP, dGTP, dTTP and/or dUTP), under a condition to generate a plurality of concatemers immobilized to the support.

In some embodiments, in the methods for conducting rolling circle amplification reaction as described herein, the plurality of the first surface primers (e.g., surface capture primers) immobilized on the support comprise the sequence 5′-GATCAGGTGAGGCTGCGACGACT-3′ (SEQ ID NO:28). Individual first surface primers (e.g., surface capture primers) can hybridize to a covalently closed circular library molecule (400) having a universal binding sequence for the first surface primer.

In some embodiments, the methods for conducting rolling circle amplification reaction further comprise step (b): contacting the plurality of immobilized covalently closed circular library molecules (400) with a plurality of strand-displacing polymerases and a plurality of nucleotides, under a condition suitable to conduct a rolling circle amplification reaction on the support using the plurality of first surface primers (e.g., surface capture primers) as immobilized amplification primers and the plurality of covalently closed circular library molecules (400) as template molecules, thereby generating a plurality of nucleic acid concatemer molecules immobilized to the first surface primers (e.g., surface capture primers). In some embodiments, the plurality of nucleotides comprises any combination of two or more of dATP, dGTP, dCTP, dTTP and/or dUTP. In some embodiments, individual immobilized concatemers are covalently joined to individual first surface primers (e.g., surface capture primers). In some embodiments, individual covalently closed circular library molecules (400) in the plurality comprise universal binding sequences for a first and second surface primer (e.g., (120) and (130) respectively) so that the rolling circle amplification reaction generates concatemer molecules having multiple tandem copies of universal binding sequences for first and second surface primers. In some embodiments, the support further comprises a plurality of second surface primers (e.g., surface pinning primers). In some embodiments, the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to one or more sequencing reactions.

In some embodiments, the plurality of the second surface primers (e.g., surface pinning primers) immobilized on the support comprise the sequence 5′-CATGTAATGC ACGTACTTTCAGGGT-3′ (SEQ ID NO:29, or a complementary sequence thereof).

Individual second surface primers can hybridize to a portion of the concatemer molecules having a universal binding sequence for the second surface primer. In some embodiments, the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to one or more sequencing reactions.

### In-Solution Rolling Circle Amplification Using Soluble Amplification Primers

In some embodiments, the plurality of covalently closed circular molecules (400) serves as template molecules in an in-solution rolling circle amplification reaction to generate a plurality of concatemer molecules. The plurality of concatemer molecules may then distributed onto a coated support to generate immobilized concatemer molecules. The immobilized concatemer molecules can be subjected to one or multiple cycles of sequencing reactions.

In some embodiments, the methods for conducting rolling circle amplification reaction on a plurality of covalently closed circular library molecules (400) (e.g., which lack hybridized single-stranded splint strands (200)), wherein individual covalently closed circular library molecules (400) in the plurality comprise a universal binding sequence for a forward amplification primer and a universal binding sequence for a first surface primer, the method comprises: step (a) hybridizing in-solution a plurality of covalently closed circular library molecules and a plurality of soluble forward amplification primers. In some embodiments, the method further comprises step (b) conducting a first rolling circle amplification reaction by contacting the plurality of covalently closed circular library molecules (400) with a plurality of strand-displacing polymerases and a plurality of nucleotides (e.g., dATP, dCTP, dGTP, dTTP and/or dUTP), under a condition suitable to conduct a rolling circle amplification reaction in solution using the plurality of forward amplification primers and the plurality of covalently closed circular library molecules (400) as template molecules, thereby generating a plurality of nucleic acid concatemer molecules. In some embodiments, a portion of the generated concatemer molecules are still hybridized to their covalently closed circular library molecules (400).

In some embodiments, the methods for conducting rolling circle amplification reaction further comprises step (c): distributing the plurality of concatemer molecules onto a support having a plurality of the first surface primers immobilized thereon, under a condition suitable for hybridizing at least a portion of the concatemers to the plurality of the immobilized first surface primers (e.g., surface capture primers) thereby immobilizing the plurality of concatemer molecules. The plurality of immobilized concatemer molecules may still be hybridized to their covalently closed circular library molecules (400).

In some embodiments, the methods for conducting rolling circle amplification reaction further comprises step (d): contacting the immobilized plurality of concatemer molecules with a plurality of strand-displacing polymerases and a plurality of nucleotides, under a condition suitable to conduct a second rolling circle amplification reaction on the support using the plurality of covalently closed circular library molecules (400) as template molecules, thereby extending the plurality of immobilized nucleic acid concatemer molecules. In some embodiments, the first and/or the second rolling circle amplification reactions can be conducted with a plurality of nucleotides which comprise any combination of two or more of dATP, dGTP, dCTP, dTTP, and/or dUTP. In some embodiments, individual immobilized concatemers are hybridized to individual first surface primers (e.g., surface capture primers). In some embodiments, individual covalently closed circular library molecules (400) in the plurality comprise universal binding sequences for a first and second surface primer (e.g., (120) and (130), respectively) so that the in-solution rolling circle amplification reaction generates concatemer molecules having multiple tandem copies of universal binding sequences for first and second surface primers. In some embodiments, the support further comprises a plurality of second surface primers (e.g., surface pinning primers). In some embodiments, the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to sequencing reactions.

In some embodiments, in the methods for conducting rolling circle amplification reaction as described herein, the plurality of the first surface primers immobilized on the support comprise the sequence 5′-GATCAGGTGAGGCTGCGACGACT-3′ (SEQ ID NO:28). In some embodiments, individual first surface primers can hybridize to a covalently closed circular library molecule (400) having a universal binding sequence for the first surface primer.

In some embodiments, the plurality of the second surface primers immobilized on the support comprise the sequence 5′-CATGTAATGCACGTACTTTCAGGGT-3′ (SEQ ID NO:29, or a complementary sequence thereof). Individual second surface primers can hybridize to a portion of the concatemer molecules having a universal binding sequence for the second surface primer.

In some embodiments, the immobilized second surface primers serve to pin down at least one portion of the concatemer molecules to the support. In some embodiments, the immobilized second surface primers have a non-extendible 3′ end and cannot be used for amplification. In some embodiments, the immobilized concatemers can be subjected to sequencing reactions.

In some embodiments, in the methods for conducting on-support or in-solution rolling circle amplification reaction, the plurality of covalently closed circular library molecules (400) can be distributed onto a support that is coated with one or more compounds to produce a passivated layer on the support (e.g., FIG. 28). In some embodiments, the passivated layer forms a porous or semi-porous layer. In some embodiments, one or more types of surface primers, concatemer template molecules and/or polymerases, can be attached to the passivated layer for immobilization to the support. In some embodiments, the support comprises a low non-specific binding surface that enables improved nucleic acid hybridization and amplification performance on the support. In some embodiments, the support may comprise one or more layers of a covalently or non-covalently attached low-binding, chemical modification layers, e.g., silane layers, polymer films, and one or more covalently or non-covalently attached oligonucleotides that can be used for immobilizing a plurality of nucleic acid concatemer molecules to the support. In some embodiments, the support comprises a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides which serve as surface capture or pinning primers. The hydrophilic polymer coating layer can comprise polyethylene glycol (PEG) or a derivative thereof. The hydrophilic polymer coating layer can comprise branched PEG having at least 4 branches. In some embodiments, the polymer coating comprises polyethylene glycol (PEG) tethered to one or more oligonucleotides which serve as surface capture or pinning primers. In some embodiments, the low non-specific binding coating has a degree of hydrophilicity which can be measured as a water contact angle, wherein the water contact angle is no more than 45 degrees. In some embodiments, the density of the covalently closed circular library molecules (400) immobilized to the support or immobilized to the coating on the support is about 10²-10⁶ per mm², about 10⁶-10⁹ per mm², or about 10⁹-10¹² per mm² (e.g., 10², 10¹, 10⁴, 10⁵, 10⁶, 10⁷, 10⁸, 10⁹, 10¹⁰, 10¹¹, or 10¹²). In some embodiments, the plurality of covalently closed circular library molecules (400) is immobilized to the support or immobilized to the coating on the support at pre-determined sites on the support (or the coating on the support). In some embodiments, the plurality of covalently closed circular library molecules (400) is immobilized to the coating on the support at random sites on the support (or the coating on the support).

In some embodiments, in the methods for conducting on-support or in-solution rolling circle amplification reaction, the step of distributing the plurality of covalently closed circular library molecules (400) onto a support can be conducted in the presence of a high-efficiency hybridization buffer which comprises: (i) a first polar aprotic solvent having a dielectric constant that is no greater than 40 (e.g., less than 10, or about 10, 15, 20, 30, or 40) and having a polarity index of 4-9 (e.g., 4, 5, 6, 7, 8, or 9); (ii) a second polar aprotic solvent having a dielectric constant that is no greater than 115 (e.g., less than 10, 10, 15, 20, 30, 40, 50, 75, 100, 105, 105, 110, or 115) and is present in the hybridization buffer formulation in an amount effective to denature double-stranded nucleic acids; (iii) a pH buffer system that maintains the pH of the hybridization buffer formulation in a range of about 4-8 (e.g., 4, 5, 6, 7, or 8); and (iv) a crowding agent in an amount sufficient to enhance or facilitate molecular crowding. In some embodiments, the high efficiency hybridization buffer comprises: (i) the first polar aprotic solvent comprises acetonitrile at 25-50% (e.g., 25%, 30%, 35%, 40%, 45%, or 50%) by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5 (e.g., about 5.0, 5.5, 6.0, or 6.5); and (iv) the crowding agent comprises polyethylene glycol (PEG) at 5-35% (e.g., 5%, 10%, 15%, 20%, 25%, 30%, or 35%) by volume of the hybridization buffer. In some embodiments, the high efficiency hybridization buffer further comprises betaine.

### Compaction Oligonucleotides

In some embodiments, the on-support or in-solution rolling circle amplification reaction can be conducted in the presence of a plurality of compaction oligonucleotides. In some embodiments, the compaction oligonucleotides comprise single stranded oligonucleotides comprising DNA, RNA, or a combination of DNA and RNA. The compaction oligonucleotides can be any length, including 20-150 nucleotides, 30-100 nucleotides, or 40-80 nucleotides in length. Compaction nucleotides may be, e.g., about 10, 15, 20, 25, 30, 35, 40, 45, 50, 60, 70, 80, 90, 100, 110, 120, 130, 140, or 150 nucleotides in length.

In some embodiments, the compaction oligonucleotide comprises a 5′ region and a 3′ region, and optionally an intervening region between the 5′ and 3′ regions. The intervening region can be any length, for example and without limitation, about 2-20 (e.g., 3, 4, 5, 6, 7, 8, 9, 10, 11, 12, 13, 14, 15, 16, 17, 18, 19, or 20 nucleotides) nucleotides in length. In some embodiments, the intervening region comprises a homopolymer having consecutive identical bases (e.g., AAA, GGG, CCC, TTT, or UUU). In some embodiments, the intervening region comprises a non-homopolymer sequence.

The 5′ region of the compaction oligonucleotides can be wholly complementary or partially complementary along its length to a first portion of a concatemer molecule. Alternatively, or additionally, the 3′ region of the compaction oligonucleotides can be wholly complementary or partially complementary along its length to a second portion of a concatemer molecule.

In some embodiments, the 5′ and 3′ regions of the compaction oligonucleotides comprise the same sequence. In some embodiments, the 5′ region has a sequence that is inverted compared to the 3′ region. The 5′ and 3′ regions of the compaction oligonucleotide can hybridize to the concatemer to pull together distal portions of the concatemer causing compaction of the concatemer to form a DNA nanoball. Inclusion of compaction oligonucleotides during RCA can promote formation of DNA nanoballs having tighter size and shape compared to concatemers generated in the absence of the compaction oligonucleotides. Without wishing to be bound by theory, it is believed that the compact and stable characteristics of the DNA nanoballs improves sequencing accuracy by increasing signal intensity and they retain their shape and size during multiple sequencing cycles.

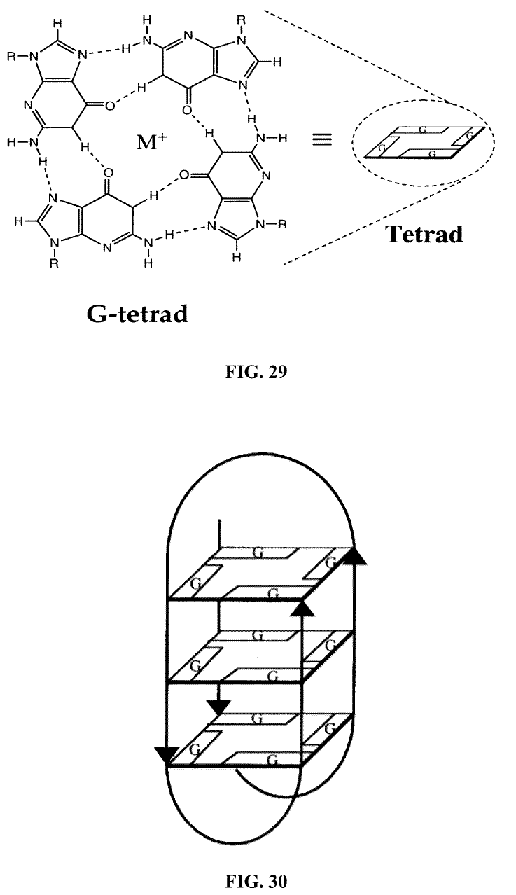

In some embodiments, the compaction oligonucleotides can include at least one region having consecutive guanines. For example, the compaction oligonucleotides can include at least one region having 2, 3, 4, 5, 6 or more consecutive guanines. In some embodiments, the compaction oligonucleotides comprise four consecutive guanines which can form a guanine tetrad structure (e.g., FIG. 29). The guanine tetrad structure may be stabilized via any suitable chemistry as known in the art. For example, the guanine tetrad structure can be stabilized via Hoogsteen hydrogen bonding. Alternatively, the guanine tetrad structure can be stabilized by a central cation including potassium, sodium, lithium, rubidium, or cesium.

In certain embodiments, at least one compaction oligonucleotide can form a guanine tetrad and hybridize to the universal binding sequences for the compaction oligonucleotide, and the resulting concatemer can fold to form an intramolecular G-quadruplex structure (e.g., FIG. 30). The concatemers can self-collapse to form compact nanoballs. It is contemplated herein that formation of the guanine tetrads and G-quadruplexes in the nanoballs may increase the stability of the nanoballs to retain their compact size and shape which can withstand repeated flows of reagents for conducting any of the sequencing workflows described herein.

### Additional Methods for Sequencing

In some aspects, the present disclosure provides methods for sequencing any of the immobilized concatemer molecules described herein. Any of the methods for conducting rolling circle amplification reaction described herein can be used to generate a plurality of concatemer molecules immobilized to a support, and the immobilized concatemers can be subjected to multiple cycles of sequencing reactions. In some embodiments, the sequencing reactions employ detectably labeled nucleotide analogs. In some embodiments, the sequencing reactions employ a two-stage sequencing reaction comprising binding detectably labeled multivalent molecules and incorporating nucleotide analogs. In some embodiments, the sequencing reactions employ non-labeled nucleotide analogs. The terms “concatemer molecule” and “template molecule” are used interchangeably herein.

In some embodiments, any of the rolling circle amplification reactions described herein (e.g., RCA conducted on-support or in-solution) can be used to generate immobilized concatemers, each concatemer containing tandem repeat units of the sequence-of-interest and any adaptor sequences present in the covalently closed circular library molecules (400). In a non-limiting example, the tandem repeat unit comprises: (i) a surface pinning primer binding site (120), (ii) a left sample index sequence (160), (iii) a forward sequencing primer binding site (140), (iv) a left UMI sequence (180), (v) an insert sequence (e.g., sequence of interest) (110), (vi) a reverse sequencing primer binding site (150), (vii) a right sample index sequence (170) which optionally includes a 3-mer random sequence, and (viii) a surface capture primer binding site (130) (e.g., see FIG. 10). In some embodiments, the immobilized concatemers comprise tandem repeat units which include one UMI sequence, for example a left UMI sequence (180) or a right UMI sequence (190). In some embodiments, the immobilized concatemers comprise tandem repeat units which include two UMI sequences, for example a left UMI sequence (180) and a right UMI sequence (190).

In some embodiments, any of the rolling circle amplification reactions described herein (e.g., RCA conducted on-support or in-solution) can be used to generate immobilized concatemers each containing tandem repeat units of the sequence-of-interest and any adaptor sequences present in the covalently closed circular library molecules (1000). In a non-limiting example, the tandem repeat unit comprises: (i) a surface pinning primer binding site (520), (ii) a left sample index sequence (560), (iii) a forward sequencing primer binding site (540), (iv) a left UMI sequence (580), (v) an insert sequence (e.g., sequence of interest) (510), (vi) a reverse sequencing primer binding site (550), (vii) a right sample index sequence (570) which optionally includes a 3-mer random sequence, and (viii) a surface capture primer binding site (530) (e.g., see FIG. 13). In some embodiments, the immobilized concatemers comprise tandem repeat units which include one UMI sequence, for example, a left UMI sequence (580) or a right UMI sequence (590). In some embodiments, the immobilized concatemers comprise tandem repeat units which include two UMI sequences, for example, a left UMI sequence (580) and a right UMI sequence (590).

The immobilized concatemer can self-collapse into a compact nucleic acid nanoball. Inclusion of one or more compaction oligonucleotides during the on-support or in-solution RCA reaction can further compact the size and/or shape of the nanoball. An increase in the number of tandem repeat units in a given concatemer may increase the number of sites along the concatemer for hybridizing to multiple sequencing primers (e.g., sequencing primers having a universal sequence) which serve as multiple initiation sites for polymerase-catalyzed sequencing reactions. When the sequencing reaction employs detectably labeled nucleotides and/or detectably labeled multivalent molecules (e.g., having nucleotide units), the signals emitted by the nucleotides or nucleotide units that participate in the parallel sequencing reactions along the concatemer may yield an increased signal intensity for each concatemer. Multiple portions of a given concatemer can be simultaneously sequenced. Furthermore, a plurality of binding complexes can form along a particular concatemer molecule, each binding complex comprising a sequencing polymerase bound to a multivalent molecule wherein the plurality of binding complexes remains stable without dissociation resulting in increased persistence time which increases signal intensity and reduces imaging time.

### Methods for Sequencing Using Nucleotide Analogs

In some embodiments, the present disclosure further provides methods for sequencing any of the immobilized concatemer molecules described herein, the methods comprising step (a): contacting a sequencing polymerase to (i) a nucleic acid concatemer molecule and (ii) a nucleic acid sequencing primer, wherein the contacting is conducted under a condition suitable to bind the sequencing polymerase to the nucleic acid concatemer molecule which is hybridized to the nucleic acid primer, wherein the nucleic acid concatemer molecule hybridized to the nucleic acid primer forms the nucleic acid duplex. In some embodiments, the sequencing polymerase comprises a recombinant mutant sequencing polymerase that can bind and incorporate nucleotide analogs. In some embodiments, the sequencing primer comprises a 3′ extendible end.

In some embodiments, in the methods for sequencing concatemer molecules, the sequencing primer comprises a 3′ extendible end or a 3′ non-extendible end. In some embodiments, the plurality of nucleic acid concatemer molecules comprise amplified template molecules (e.g., clonally amplified template molecules). In some embodiments, the plurality of nucleic acid concatemer molecules comprise one copy of a target sequence of interest. In some embodiments, the plurality of nucleic acid molecules comprises two or more tandem copies of a target sequence of interest (e.g., concatemers). In some embodiments, the nucleic acid concatemer molecules in the plurality of nucleic acid concatemer molecules comprise the same target sequence of interest or different target sequences of interest. In some embodiments, the plurality of nucleic acid concatemer molecules and/or the plurality of nucleic acid primers are in solution or are immobilized to a support. In some embodiments, when the plurality of nucleic acid concatemer molecules and/or the plurality of nucleic acid primers are immobilized to a support, the binding with the first sequencing polymerase generates a plurality of immobilized first complexed polymerases. In some embodiments, the plurality of nucleic acid concatemer molecules and/or nucleic acid primers are immobilized to 10²-10¹⁵ different sites (e.g., 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, or 10¹⁵ sites) on a support. In some embodiments, the binding of the plurality of concatemer molecules and nucleic acid primers with the plurality of first sequencing polymerases generates a plurality of first complexed polymerases immobilized to 10²-10¹⁵ different sites (e.g., 10² sites, 10³ sites, 10⁴ sites, 10⁵ sites, 10⁶ sites, 10⁷ sites, 10⁸ sites, 10⁹ sites, 10¹⁰ sites, 10¹¹ sites, 10¹² sites, 10¹³ sites, 10¹⁴ sites, or 10¹⁵ sites) on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

In some embodiments, the methods for sequencing further comprise step (b): contacting the sequencing polymerase with a plurality of nucleotides under a condition suitable for binding at least one nucleotide to the sequencing polymerase which is bound to the nucleic acid duplex and suitable for polymerase-catalyzed nucleotide incorporation. In some embodiments, the sequencing polymerase is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least one nucleotide is labeled with a detectable reporter moiety (e.g., fluorophore).

In some embodiments, the methods for sequencing further comprise step (c): incorporating at least one nucleotide into the 3′ end of the extendible primer under a condition suitable for incorporating the at least one nucleotide. In some embodiments, the suitable conditions for nucleotide binding the polymerase and for incorporation the nucleotide can be the same or different. In some embodiments, conditions suitable for incorporating the nucleotide comprise inclusion of at least one catalytic cation comprising magnesium and/or manganese. In some embodiments, the at least one nucleotide binds the sequencing polymerase and incorporates into the 3′ end of the extendible primer. In some embodiments, the incorporating the nucleotide into the 3′ end of the primer in step (c) comprises a primer extension reaction.

In some embodiments, the methods for sequencing further comprise step (d): repeating the incorporating at least one nucleotide into the 3′ end of the extendible primer of steps (b) and (c) at least once. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base. In some embodiments, the method further comprises detecting the at least one incorporated nucleotide at step (c) and/or (d). In some embodiments, the method further comprises identifying the at least one incorporated nucleotide at step (c) and/or (d). In some embodiments, the sequence of the nucleic acid concatemer molecule can be determined by detecting and identifying the nucleotide that binds the sequencing polymerase, thereby determining the sequence of the concatemer molecule. In some embodiments, the sequence of the nucleic acid concatemer molecule can be determined by detecting and identifying the nucleotide that incorporates into the 3′ end of the primer, thereby determining the sequence of the concatemer molecule.

In some embodiments, in the methods for sequencing described herein, the plurality of sequencing polymerases that are bound to the nucleic acid duplexes comprise a plurality of complexed polymerases, having at least a first and second complexed polymerase, wherein (a) the first complexed polymerases comprises a first sequencing polymerase bound to a first nucleic acid duplex comprising a first nucleic acid template sequence which is hybridized to a first nucleic acid primer, (b) the second complexed polymerases comprises a second sequencing polymerase bound to a second nucleic acid duplex comprising a second nucleic acid template sequence which is hybridized to a second nucleic acid primer, (c) the first and second nucleic acid template sequences comprise the same or different sequences, (d) the first and second nucleic acid concatemers are clonally-amplified, (e) the first and second primers comprise extendible 3′ ends or non-extendible 3′ ends, and (f) the plurality of complexed polymerases are immobilized to a support. In some embodiments, the density of the plurality of complexed polymerases is about 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) complexed polymerases per mm² that are immobilized to the support.

### Two-Stage Methods for Nucleic Acid Sequencing

In some aspects, the present disclosure provides a two-stage method for sequencing any of the immobilized concatemer molecules described herein. In some embodiments, the first stage generally comprises binding multivalent molecules to complexed polymerases to form multivalent-complexed polymerases and detecting the multivalent-complexed polymerases.

In some embodiments, the first stage comprises step (a): contacting a plurality of a first sequencing polymerase to (i) a plurality of nucleic acid concatemer molecules and (ii) a plurality of nucleic acid sequencing primers. In some embodiments, the contacting is conducted under a condition suitable to bind the plurality of first sequencing polymerases to the plurality of nucleic acid concatemer molecules and the plurality of nucleic acid primers, thereby forming a plurality of first complexed polymerases each comprising a first sequencing polymerase bound to a nucleic acid duplex wherein the nucleic acid duplex comprises a nucleic acid concatemer molecule hybridized to a nucleic acid primer. In some embodiments, the first polymerase comprises a recombinant mutant sequencing polymerase. In some embodiments, the sequencing primer comprises a 3′ extendible end.

In some embodiments, in the methods for sequencing concatemer molecules as described herein, the sequencing primer comprises a 3′ extendible end. Alternatively, the sequencing primer comprises a 3′ non-extendible end. In some embodiments, the plurality of nucleic acid concatemer molecules comprise amplified template molecules (e.g., clonally amplified template molecules). In some embodiments, the plurality of nucleic acid concatemer molecules comprise one copy of a target sequence of interest. In some embodiments, the plurality of nucleic acid molecules comprises two or more tandem copies of a target sequence of interest (e.g., concatemers). In some embodiments, the nucleic acid concatemer molecules in the plurality of nucleic acid concatemer molecules comprise the same target sequence of interest or different target sequences of interest. In some embodiments, the plurality of nucleic acid concatemer molecules and/or the plurality of nucleic acid primers are in solution or are immobilized to a support. In some embodiments, when the plurality of nucleic acid concatemer molecules and/or the plurality of nucleic acid primers are immobilized to a support, the binding with the first sequencing polymerase generates a plurality of immobilized first complexed polymerases. In some embodiments, the plurality of nucleic acid concatemer molecules and/or nucleic acid primers are immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) different sites on a support. In some embodiments, the binding of the plurality of concatemer molecules and nucleic acid primers with the plurality of first sequencing polymerases generates a plurality of first complexed polymerases immobilized to 10²-10¹⁵ (e.g., 10², 10³, 10⁴, 10⁵, 10⁶, 10⁷, 10⁸, 10⁹, 10¹⁰, 10¹¹, 10¹², 10¹³, 10¹⁴, or 10¹⁵) different sites on the support. In some embodiments, the plurality of immobilized first complexed polymerases on the support are immobilized to pre-determined or to random sites on the support. In some embodiments, the plurality of immobilized first complexed polymerases are in fluid communication with each other to permit flowing a solution of reagents (e.g., enzymes including sequencing polymerases, multivalent molecules, nucleotides, and/or divalent cations) onto the support so that the plurality of immobilized complexed polymerases on the support are reacted with the solution of reagents in a massively parallel manner.

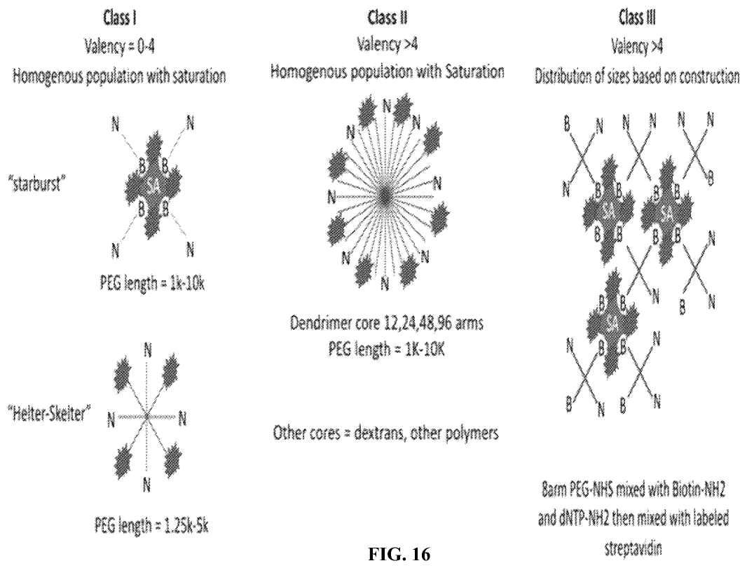

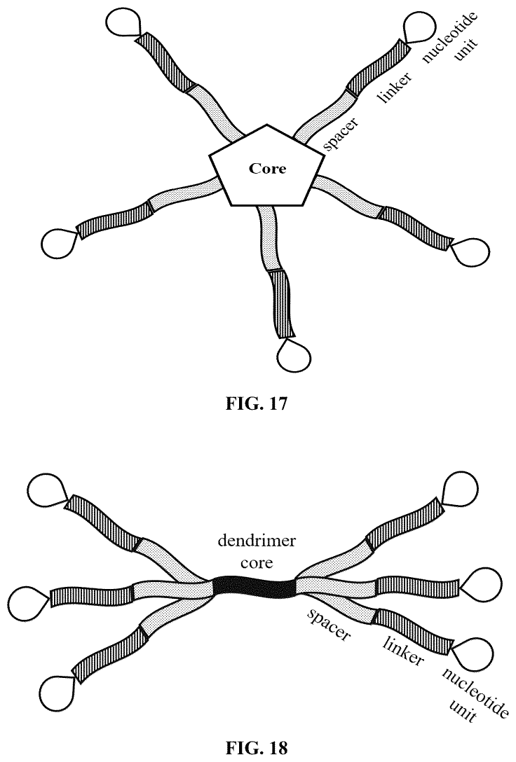

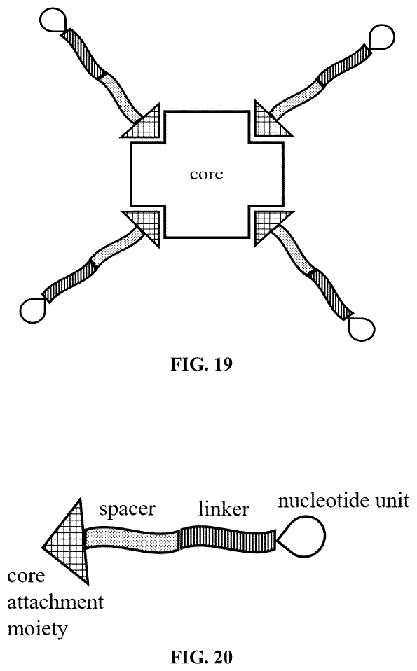

In some embodiments, the methods for sequencing further comprise step (b): contacting the plurality of first complexed polymerases with a plurality of multivalent molecules to form a plurality of multivalent-complexed polymerases (e.g., binding complexes). In some embodiments, individual multivalent molecules in the plurality of multivalent molecules comprise a core attached to multiple nucleotide arms and each nucleotide arm is attached to a nucleotide (e.g., nucleotide unit) (e.g., FIGS. 16-20). In some embodiments, the contacting of step (b) is conducted under a condition suitable for binding complementary nucleotide units of the multivalent molecules to at least two of the plurality of first complexed polymerases thereby forming a plurality of multivalent-complexed polymerases. In some embodiments, the condition is suitable for inhibiting polymerase-catalyzed incorporation of the complementary nucleotide units into the primers of the plurality of multivalent-complexed polymerases. In some embodiments, the plurality of multivalent molecules comprises at least one multivalent molecule having multiple nucleotide arms (e.g., FIGS. 16-19) each attached with a nucleotide analog (e.g., nucleotide analog unit), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide unit that lacks a chain terminating moiety. In some embodiments, at least one of the multivalent molecules in the plurality of multivalent molecules is labeled with a detectable reporter moiety. In some embodiments, the detectable reporter moiety comprises a fluorophore. In some embodiments, the contacting of step (b) is conducted in the presence of at least one non-catalytic cation comprising strontium, barium and/or calcium.

In some embodiments, the methods for sequencing further comprise step (c): detecting the plurality of multivalent-complexed polymerases. In some embodiments, the detecting includes detecting the multivalent molecules that are bound to the complexed polymerases, where the complementary nucleotide units of the multivalent molecules are bound to the primers, but incorporation of the complementary nucleotide units is inhibited. In some embodiments, the multivalent molecules are labeled with a detectable reporter moiety to permit detection. In some embodiments, the labeled multivalent molecules comprise a fluorophore attached to the core, linker and/or nucleotide unit of the multivalent molecules.

In some embodiments, the methods for sequencing further comprise step (d): identifying the nucleo-base of the complementary nucleotide units that are bound to the plurality of first complexed polymerases, thereby determining the sequence of the concatemer molecule. In some embodiments, the multivalent molecules are labeled with a detectable reporter moiety that corresponds to the particular nucleotide units attached to the nucleotide arms to permit identification of the complementary nucleotide units (e.g., nucleotide base adenine, guanine, cytosine, thymine, or uracil) that are bound to the plurality of first complexed polymerases.

In some embodiments, the second stage of the two-stage sequencing method generally comprises nucleotide incorporation. In some embodiments, the methods for sequencing further comprise step (e): dissociating the plurality of multivalent-complexed polymerases and removing the plurality of first sequencing polymerases and their bound multivalent molecules and retaining the plurality of nucleic acid duplexes.

In some embodiments, the methods for sequencing further comprise step (f): contacting the plurality of the retained nucleic acid duplexes of step (e) with a plurality of second sequencing polymerases. In some embodiments, the contacting is conducted under a condition suitable for binding the plurality of second sequencing polymerases to the plurality of the retained nucleic acid duplexes, thereby forming a plurality of second complexed polymerases each comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) has an amino acid sequence that is 100% identical to the amino acid sequence as the plurality of the second sequencing polymerases of step (f). In some embodiments, the plurality of first sequencing polymerases of step (a) has an amino acid sequence that differs from the amino acid sequence of the plurality of the second sequencing polymerases of step (f).

In some embodiments, the methods for sequencing further comprise step (g): contacting the plurality of second complexed polymerases with a plurality of nucleotides. In some embodiments, the contacting is conducted under a condition suitable for binding complementary nucleotides from the plurality of nucleotides to at least two of the second complexed polymerases thereby forming a plurality of nucleotide-complexed polymerases. In some embodiments, the contacting of step (g) is conducted under a condition that is suitable for promoting polymerase-catalyzed incorporation of the bound complementary nucleotides into the primers of the nucleotide-complexed polymerases, thereby forming a plurality of nucleotide-complexed polymerases. In some embodiments, the incorporating the nucleotide into the 3′ end of the primer in step (g) comprises a primer extension reaction. In some embodiments, the contacting of step (g) is conducted in the presence of at least one catalytic cation comprising magnesium and/or manganese. In some embodiments, at least one of the nucleotides in the plurality is not labeled with a detectable reporter moiety. In some embodiments, the plurality of nucleotides comprises non-labeled nucleotides. In some embodiments, the plurality of nucleotides comprises native nucleotides (e.g., non-analog nucleotides) or nucleotide analogs. In some embodiments, the plurality of nucleotides comprises a 2′ and/or 3′ chain terminating moiety which is removable. Alternatively, in some embodiments, the 2′ and/or 3′ chain terminating moiety is not removable. In some embodiments, the plurality of nucleotides comprises a plurality of nucleotides labeled with detectable reporter moiety. The detectable reporter moiety may comprise a fluorophore. In some embodiments, the fluorophore is attached to the nucleotide base. In some embodiments, the fluorophore is attached to the nucleotide base with a linker which is cleavable and/or otherwise removable from the base. In some embodiments, the fluorophore is not removable from the base. In some embodiments, a particular detectable reporter moiety (e.g., fluorophore) that is attached to the nucleotide can correspond to the nucleotide base (e.g., dATP, dGTP, dCTP, dTTP, or dUTP) to permit detection and identification of the nucleotide base.

In some embodiments, the methods for sequencing further comprise step (h): detecting the complementary nucleotides which are incorporated into the primers of the nucleotide-complexed polymerases. In some embodiments, the plurality of nucleotides is labeled with a detectable reporter moiety to permit detection. In some embodiments, in the methods for sequencing concatemer molecules, when the plurality of nucleotides in step (g) are non-labeled, the detecting of step (h) is omitted.

In some embodiments, the methods for sequencing further comprise step (i): identifying the bases of the complementary nucleotides which are incorporated into the primers of the nucleotide-complexed polymerases. In some embodiments, the identification of the incorporated complementary nucleotides in step (i) can be used to confirm the identity of the complementary nucleotides of the multivalent molecules that are bound to the plurality of first complexed polymerases in step (d). In some embodiments, the identifying of step (i) can be used to determine the sequence of the nucleic acid concatemer molecules. In some embodiments, in the methods for sequencing concatemer molecules, when the plurality of nucleotides in step (g) are non-labeled, the identifying of step (i) is omitted.

In some embodiments, the methods for sequencing further comprise step (j): removing the chain terminating moiety from the incorporated nucleotide when step (g) is conducted by contacting the plurality of second complexed polymerases with a plurality of nucleotides that comprise at least one nucleotide having a 2′ and/or 3′ chain terminating moiety.

In some embodiments, the methods for sequencing further comprise step (k): repeating steps (a)-(j) at least once. In some embodiments, the sequence of the nucleic acid concatemer molecules can be determined by detecting and identifying the multivalent molecules that bind the sequencing polymerases but do not incorporate into the 3′ end of the primer at steps (c) and (d). In some embodiments, the sequence of the nucleic acid concatemer molecule can be determined (or confirmed) by detecting and identifying the nucleotide that incorporates into the 3′ end of the primer at steps (h) and (i). In some embodiments, steps (a)-(j) are performed in order.

In some embodiments, in any of the methods for sequencing nucleic acid molecules, the binding of the plurality of first complexed polymerases with the plurality of multivalent molecules forms at least one avidity complex, the method comprises the steps: (a) binding a first nucleic acid primer, a first sequencing polymerase, and a first multivalent molecule to a first portion of a concatemer template molecule thereby forming a first binding complex, wherein a first nucleotide unit of the first multivalent molecule binds to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule binds to the second sequencing polymerase, wherein the first and second binding complexes which include the same multivalent molecule forms an avidity complex. In some embodiments, the first sequencing polymerase comprises any wild type or mutant polymerase described herein. In some embodiments, the second sequencing polymerase comprises any wild type or mutant polymerase described herein. In some embodiments, the concatemer template molecule comprises tandem repeat sequences of a sequence of interest and at least one universal sequencing primer binding site. The first and second nucleic acid primers can bind to a sequencing primer binding site along the concatemer template molecule. Exemplary multivalent molecules are shown in FIGS. 16-19.

In some embodiments, in any of the methods for sequencing nucleic acid molecules described herein, wherein the method includes binding the plurality of first complexed polymerases with the plurality of multivalent molecules to form at least one avidity complex, the method comprising the steps: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases. In some embodiments, at least a first nucleotide unit of the single multivalent molecule is bound to the first complexed polymerase which includes a first primer hybridized to a first portion of the concatemer template molecule thereby forming a first binding complex (e.g., first ternary complex). In some embodiments, at least a second nucleotide unit of the single multivalent molecule is bound to the second complexed polymerase which includes a second primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide units in the first and second binding complexes. In some embodiments, the first and second binding complexes which are bound to the same multivalent molecule forms an avidity complex. In some embodiments, the methods comprise step (c) detecting the first and second binding complexes on the same concatemer template molecule, and step (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the plurality of sequencing polymerases comprise any wild type or mutant sequencing polymerase described herein. The concatemer template molecule may comprise tandem repeat sequences of a sequence of interest and at least one universal sequencing primer binding site. The plurality of nucleic acid primers can bind to a sequencing primer binding site along the concatemer template molecule. Exemplary multivalent molecules are shown in FIGS. 16-19.

### Sequencing-by-Binding

In some aspects, the present disclosure provides methods for sequencing any of the immobilized concatemer molecules described herein, wherein the sequencing methods comprise a sequencing-by-binding (SBB) procedure which employs non-labeled chain-terminating nucleotides. In some embodiments, the sequencing-by-binding (SBB) method comprises the steps of (a) sequentially contacting a primed template nucleic acid with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (the contents of both patents are hereby incorporated by reference in their entireties).

### Sequencing Polymerases

In some aspects, the present disclosure provides methods for sequencing nucleic acid molecules, where any of the sequencing methods described herein employ at least one type of sequencing polymerase and a plurality of nucleotides, or employ at least one type of sequencing polymerase and a plurality of nucleotides and a plurality of multivalent molecules. In some embodiments, the sequencing polymerase(s) is/are capable of incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprises recombinant mutant polymerases.

Examples of suitable polymerases for use in sequencing with nucleotides and/or multivalent molecules include, but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT®, DEEP VENT®, THERMINATOR™, Pfu, KOD, Pfx, Tgo and RB69 polymerases. It is contemplated that any suitable polymerase as known in the art may be used in the methods disclosed herein.

### Nucleotides

In some aspects, the present disclosure provides methods for sequencing nucleic acid molecules, where any of the sequencing methods described herein employ at least one nucleotide. The nucleotides generally comprise a base, sugar and at least one phosphate group. In some embodiments, at least one nucleotide in the plurality comprises an aromatic base, a five-carbon sugar (e.g., ribose or deoxyribose), and one or more phosphate groups (e.g., 1-10 phosphate groups). The plurality of nucleotides can comprise at least one type of nucleotide selected from the group consisting of dATP, dGTP, dCTP, dTTP, and dUTP. The plurality of nucleotides can comprise a mixture of any combination of two or more types of nucleotides selected from the group consisting of dATP, dGTP, dCTP, dTTP, and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing nucleic acid molecules described herein, at least one nucleotide in the plurality of nucleotides comprises a chain of one, two, or three phosphorus atoms. The chain of phosphorus atoms is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene, or ethylene. In some embodiments, the phosphorus atoms in the chain include substituted side groups including O, S, or BH₃. In some embodiments, the chain includes phosphate groups substituted with analogs including phosphoramidate, phosphorothioate, phosphordithioate, and O-methylphosphoroamidite groups.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl, or acetal group. In some embodiments, the chain terminating moiety is cleavable and/or otherwise removable from the nucleotide. The chain terminating moiety may be removable, for example and without limitation, by reacting the chain terminating moiety with a chemical agent, pH change, light, or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and disulfide are cleavable with phosphine or with a thiol group, e.g., beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido, or azidomethyl group. In some embodiments, the chain terminating moiety comprises a 3′-O-azido or 3′-O-azidomethyl group. In some embodiments, the chain terminating moieties azide, azido, and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing nucleic acid molecules described herein, the nucleotide comprises a chain terminating moiety which is selected from a group consisting of 3′-deoxy nucleotides, 2′, 3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydryl, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, 3-O-benzyl and 3′-O-acetal, or derivatives thereof.

In some embodiments, in any of the methods for sequencing nucleic acid molecules described herein, the plurality of nucleotides comprises a plurality of nucleotides labeled with one or more detectable reporter moieties. The detectable reporter moiety may comprise a fluorophore. In some embodiments, the fluorophore is attached to the nucleotide base. In some embodiments, the fluorophore is attached to the nucleotide base with a linker which is cleavable and/or otherwise removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP, or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, silyl, or acetal group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, and/or disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing nucleic acid molecules described herein, the cleavable linker on the nucleotide base comprises a cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP). In some embodiments, the chain terminating moiety comprising one or more of a 3′-O-amino group, a 3′-O-aminomethyl group, a 3′-O-methylamino group, or derivatives thereof may be cleaved with nitrous acid, for example, through a mechanism utilizing nitrous acid, or using a solution comprising nitrous acid. In some embodiments, the chain terminating moiety comprising one or more of a 3′-O-amino group, a 3′-O-aminomethyl group, a 3′-O-methylamino group, or derivatives thereof may be cleaved using a solution comprising nitrite. In some embodiments, for example, nitrite may be combined with or contacted with an acid such as acetic acid, sulfuric acid, or nitric acid. In some further embodiments, for example, nitrite may be combined with or contacted with an organic acid such as, for example, formic acid, acetic acid, propionic acid, butyric acid, isobutyric acid, or the like. In some embodiments, the chain terminating moiety comprises a 3′-acetal moiety which can be cleaved with a palladium deblocking reagent (e.g., Pd(0)).

In some embodiments, in any of the methods for sequencing nucleic acid molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

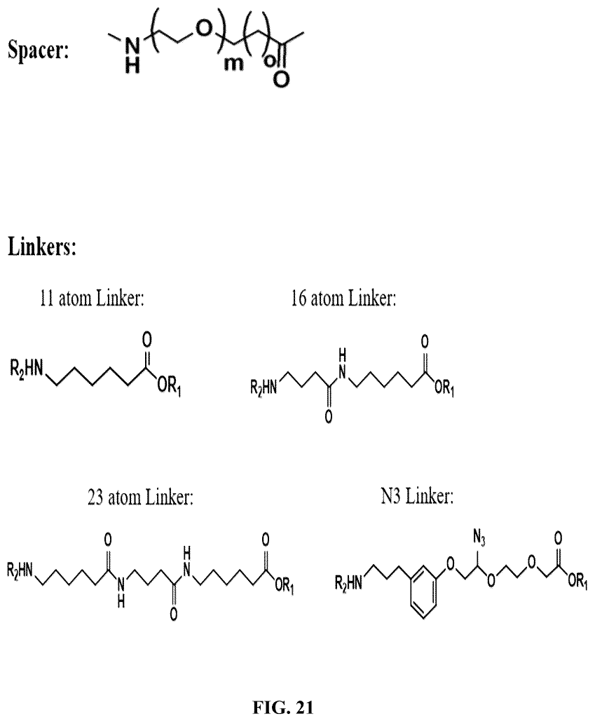

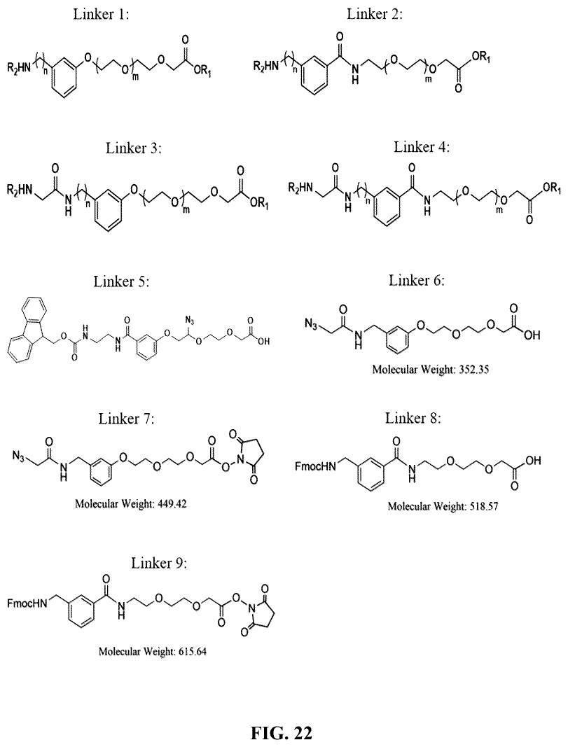

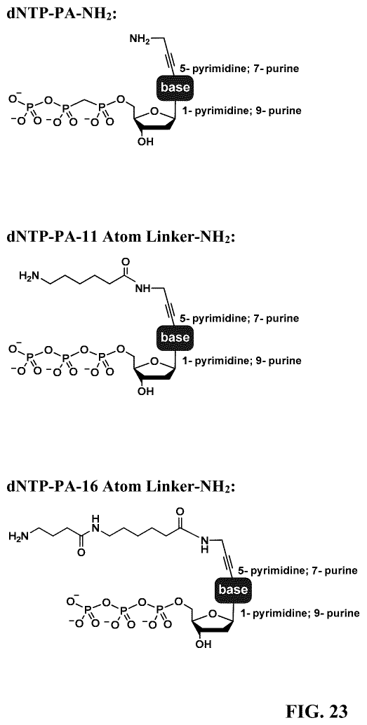

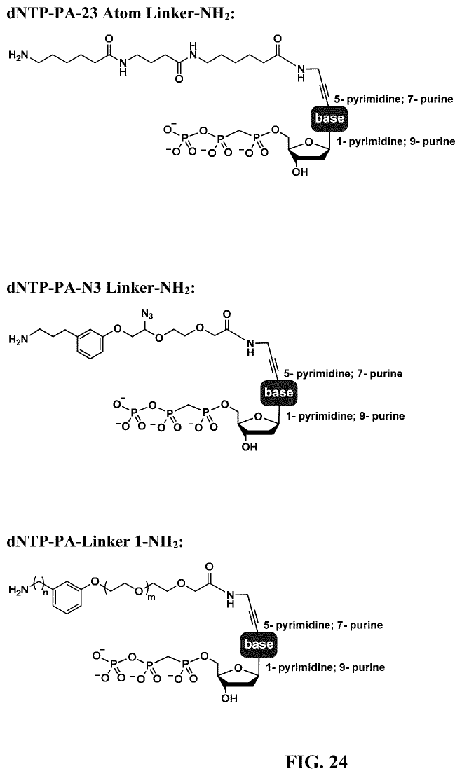

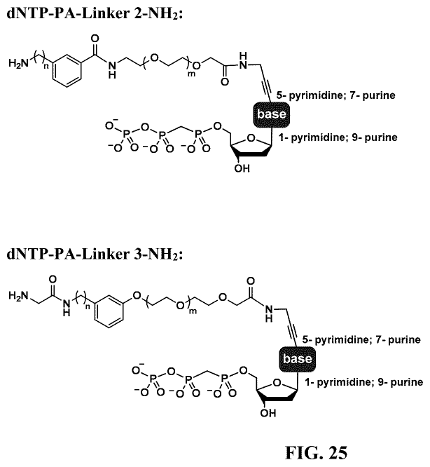

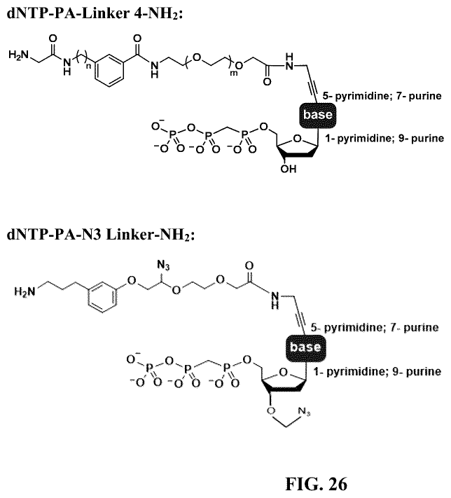

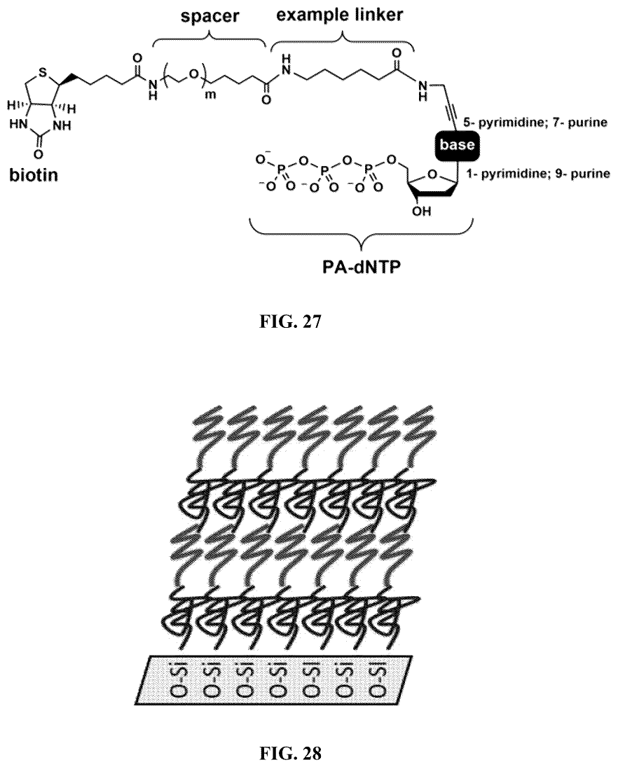

In some aspects, the present disclosure provides methods for sequencing nucleic acid molecules, where any of the sequencing methods described herein employs at least one multivalent molecule. In some embodiments, the multivalent molecule comprises a plurality of nucleotide arms attached to a core and having any configuration including a starburst, helter skelter, or bottle brush configuration (e.g., FIGS. 16-19). The multivalent molecule may comprise: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the nucleotide unit comprises a base, sugar and at least one phosphate group, and the linker is attached to the nucleotide unit through the base. In some embodiments, the linker comprises an aliphatic chain or an oligo ethylene glycol chain where both linker chains having 2-6 (e.g., 2, 3, 4, 5, or 6) subunits. In some embodiments, the linker also includes an aromatic moiety. An exemplary nucleotide arm is shown in FIG. 20. Exemplary multivalent molecules are shown in FIGS. 16-19. An exemplary spacer is shown in FIG. 21 (top) and exemplary linkers are shown in FIG. 21 (bottom) and FIG. 22. Exemplary nucleotides attached to a linker are shown in FIGS. 23-26. An exemplary biotinylated nucleotide arm is shown in FIG. 27.

In some embodiments, a multivalent molecule comprises a core attached to multiple nucleotide arms, and the multiple nucleotide arms have the same type of nucleotide unit, which is selected from the group consisting of dATP, dGTP, dCTP, dTTP, and dUTP.

In some embodiments, a multivalent molecule comprises a core attached to multiple nucleotide arms, where each arm includes a nucleotide unit. The nucleotide unit comprises an aromatic base, a five-carbon sugar (e.g., ribose or deoxyribose), and one or more phosphate groups (e.g., 1-10 phosphate groups). The plurality of multivalent molecules can comprise one type of multivalent molecule having one type of nucleotide unit selected from the group consisting of dATP, dGTP, dCTP, dTTP, and dUTP. The plurality of multivalent molecules can comprise a mixture of any combination of two or more types of multivalent molecules, where individual multivalent molecules in the mixture comprise nucleotide units selected from a group consisting of dATP, dGTP, dCTP, dTTP, and/or dUTP.

In some embodiments, the nucleotide unit comprises a chain of one, two or three phosphorus atoms, where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene, or ethylene. In some embodiments, the phosphorus atoms in the chain include substituted side groups, e.g., O, S or BH₃. In some embodiments, the chain includes phosphate groups substituted with analogs, e.g., phosphoramidate, phosphorothioate, phosphordithioate, and O-methylphosphoroamidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, silyl, or acetal group. In some embodiments, the chain terminating moiety is cleavable and/or otherwise removable from the nucleotide unit, for example and without limitation, by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, and disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydryl, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker, and nucleotide unit, and wherein the core, linker and/or nucleotide unit is labeled with a detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core comprises a streptavidin-type or avidin-type moiety which includes an avidin protein, as well as any derivatives, analogs and other non-native forms of avidin that can bind to at least one biotin moiety. Other forms of avidin moieties may include native and recombinant avidin and streptavidin as well as derivatized molecules, e.g., non-glycosylated avidin and truncated streptavidins. For example, and without limitation, an avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially available products EXTRAVIDIN®, CAPTAVIDIN™, NEUTRAVIDIN, and NEUTRALITE AVIDIN.

In some embodiments, any of the methods for sequencing nucleic acid molecules described herein can include forming a binding complex, where the binding complex comprises (i) a polymerase, a nucleic acid concatemer molecule duplexed with a primer, and a nucleotide, or the binding complex comprises (ii) a polymerase, a nucleic acid concatemer molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds. In some embodiments, the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. In some embodiments, the binding complex (e.g., ternary complex) remains stable until subjected to a condition that causes dissociation of interactions between any of the polymerase, template molecule, primer and/or the nucleotide unit or the nucleotide. For example, and without limitation, a dissociating condition may comprise contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid and destabilizes the binding complex when the nucleotide or nucleotide unit is not complementary to the next base of the template nucleic acid.

### CappableSeq Workflow

In some aspects, the present disclosure provides methods for conducting a CappableSeq workflow, for example, as described in U.S. Pat. No. 10,428,368 (incorporated by reference in its entirety) and Ettwiller et al., 2016 BMC Genomics 17:199, ‘A novel enrichment strategy reveals unprecedented number of novel transcription start sites at single base resolution in a model prokaryote and gut microbiome’ (incorporated by reference in its entirety). In some embodiments, the present disclosure provides methods for appending an affinity tag to RNA molecules, comprising step (a) providing a plurality of RNA molecules. In some embodiments, at least one of the pluralities of RNA molecules has a 5′ dephosphorylated or a 5′-triphosphorylated end. In some embodiments, the plurality of RNA molecules comprises a mixture of RNA molecules having 5′ dephosphorylated ends, 5′-triphosphorylated ends and/or non-phosphorylated ends. In some embodiments, the plurality of RNA molecules comprises one type or a mixture of different types of RNA. In some embodiments, the plurality of RNA molecules comprises prokaryotic RNA, eukaryotic RNA and/or viral RNA. In some embodiments, the RNA can be isolated from any organism including human, simian, ape, canine, feline, bovine, equine, murine, porcine, caprine, lupine, ranine, piscine, plant, insect, bacteria, and/or virus. In some embodiments, the RNA can be isolated from organisms borne in air, water, soil or food. In some embodiments, the RNA can be isolated from a mixture of organisms of the same species or sub-species. In some embodiments, the RNA can be isolated from different organisms grown in the same growth medium or grown in different growth mediums. In some embodiments, the mixture of RNA can include similar ratios or different ratios of the different RNAs.

In some embodiments, the methods for appending an affinity tag to RNA molecules, further comprise step (b): contacting the plurality of RNA molecules with a modified guanosine monophosphate nucleotide (GMP) in the presence of a capping enzyme to generate a plurality of RNA molecules capped at their 5′ ends and carrying an affinity moiety. In some embodiments, the modified GMP nucleotide comprises a modified guanosine triphosphate nucleotide. In some embodiments, the modified GMP nucleotide comprises an affinity moiety. In some embodiments, the affinity moiety comprises biotin, desthiobiotin, bis-biotin, avidin, streptavidin, protein A, maltose-binding protein, poly-histidine, HA-tag, c-myc tag, FLAG-tag, SNAP-tag, S-tag, or glutathione-S-transferase (GST). In some embodiments, the modified GMP nucleotide comprises 3′-O-(2-aminoethylcarbamoyl) (EDA)-biotin guanosine triphosphate (GTP) or 3′-desthiobiotin-tetraethylene glycol (TEG)-GTP, or (3′-desthiobiotin-TEG-guanosine 5′ triphosphate) (e.g., DTBGTP). In some embodiments, the capping enzyme can add a cap structure to the 5′ end of the RNA molecules. In some embodiments, the capping enzyme comprises a plurality of activities including an RNA triphosphatase activity, a guanylyltransferase activity and a guanine methyltransferase activity. In some embodiments, the capping enzyme can add a 7-methylguanylate cap structures (Cap 0) to the 5′end of the RNA molecules. In some embodiments, the capping enzyme can catalyze adding a m7Gppp5′N (Cap 0 structure to 5′ triphosphate RNA. In some embodiments, the capping enzyme comprises a Vaccinia Capping Enzyme (VCE) (e.g., from New England Biolabs, Ipswich, Mass.), a Bluetongue Virus capping enzyme, a Chlorella Virus capping enzyme, or a Saccharomyces cerevisiae capping enzyme. In some embodiments, the RNA molecules are contacted with a modified guanosine monophosphate nucleotide (GMP) in the presence of a capping enzyme under a condition suitable for appending (capping) the 5′ end of the RNA molecules with the modified GMP nucleotide. In some embodiments, the modified guanosine monophosphate nucleotide (GMP) comprises 3′-desthiobiotin-TEG-guanosine 5′ triphosphate) (e.g., DTBGTP), and the capping enzyme comprises Vaccinia Capping Enzyme (VCE).

In some embodiments, the methods for appending an affinity tag to RNA molecules, further comprise step (c): fragmenting the plurality of plurality of RNA molecules from step (b). In some embodiments, the fragmented RNA molecules are about 50-500 bases in length, or about 500-1500 bases in length, or about 1500-2500 bases in length, or longer lengths up to 10,000 bases in length. In the population of fragmented RNA molecules, some are capped at their 5′ ends and carrying an affinity moiety, while some lack a 5′ cap and affinity moiety.

In some embodiments, the methods for appending an affinity tag to RNA molecules, further comprise step (d): contacting the fragmented RNA molecules with a capture moiety that binds the affinity moiety attached to some of the fragmented RNA molecules to generate captured RNA molecules. In some embodiments, the capture moiety comprises a biotin, desthiobiotin, bis-biotin, avidin, streptavidin, protein A, maltose-binding protein, poly-histidine, HA-tag, c-myc tag, FLAG-tag, SNAP-tag, S-tag, or glutathione-S-transferase (GST). In some embodiments, the capture moiety is attached to a bead. In some embodiments, the bead comprises a magnetic or paramagnetic bead. In some embodiments, the capture moiety comprises streptavidin attached to paramagnetic beads.

In some embodiments, the methods for appending an affinity tag to RNA molecules, further comprise step (e): removing the non-captured RNA molecules to generate an enriched population of captured RNA molecules attached to the capture moiety. In some embodiments, the removing includes washing away the non-captured RNA molecules.

In some embodiments, the methods for appending an affinity tag to RNA molecules, further comprise step (f): eluting the captured RNA molecules from the capture moiety (e.g., from the beads) to generate a population of eluted RNA molecules.

In some embodiments, the methods for appending an affinity tag to RNA molecules, further comprise step (g): removing the 5′ cap from the eluted RNA molecules. In some embodiments, the removing step comprises contacting the eluted RNA molecules with RNA 5′ pyrophosphohydrolase (RppH) to remove the pyrophosphate from the 5′ ends of the triphosphorylated RNA thereby generating 5′ monophosphate RNA molecules. In some embodiments, the 5′ monophosphate RNA molecules can be appended with a nucleic acid adaptor at one or both ends to generate a plurality of nucleic acid library molecules.

In some embodiments, the methods for appending an affinity tag to RNA molecules, further comprise step (h): appending a first universal adaptor to one end of the RNA molecules. In some embodiments, the appending comprises ligating a single-stranded or double-stranded universal adaptor to the 5′ ends of the RNA molecules to generate adaptor-RNA molecules. In some embodiments, the ligation reaction comprises a T4 RNA ligase 1 or T4 RNA ligase 2. In some embodiments, the appending comprises employing primer extension or PCR to append a universal adaptor to the 5′ ends of the RNA molecules to generate adaptor-RNA molecules. In some embodiments, the appended universal adaptor sequence includes a unique molecular index sequence.

In some embodiments, the methods for appending an affinity tag to RNA molecules, further comprise step (i): converting the adaptor-RNA molecules to a plurality of cDNA molecules having a universal adaptor. In some embodiments, the converting comprises contacting the adaptor-RNA molecules with a reverse transcriptase enzyme. In some embodiments, the plurality of cDNA molecules can be subjected to PCR.

In some embodiments, the methods for appending an affinity tag to RNA molecules, further comprise step (j): appending a second universal adaptor to one end of the cDNA molecules to generate a plurality of adaptor-insert-adaptor molecules having a cDNA sequence of interest flanked on one side by a first universal adaptor sequence and flanked on the other side by a second universal adaptor sequence. In some embodiments, the first universal adaptor sequence comprises a first or second sequencing primer binding site. In some embodiments, the second universal adaptor sequence comprises a second or first sequencing primer binding site. In some embodiments, the method further comprises appending a third and fourth universal adaptor sequence to the adaptor-insert-adaptor molecules to generate a library molecule. In certain embodiments, the library molecule has a surface pinning primer binding site, a first sample index, a first sequencing primer binding site, a unique molecular index sequence, an insert sequence of interest, a second sequencing primer binding site, a second sample index, and a surface capture binding site.

In some embodiments, the appending of step (j) comprises ligating the second universal adaptor to the cDNA molecules to generate the adaptor-insert-adaptor molecules. In some embodiments, the appending of step (j) comprises employing primer extension or PCR to append the second universal adaptor to the cDNA molecules to generate the adaptor-insert-adaptor molecules. In some embodiments, the appended universal adaptor sequence includes a unique molecular index sequence. In some embodiments, the plurality of adaptor-insert-adaptor molecules are single-stranded DNA molecules.

In some embodiments, the methods for appending an affinity tag to RNA molecules, further comprise step (k): generating a plurality of library-splint complexes by (1) hybridizing the plurality of DNA library molecules of step (j) to a plurality of single-stranded splint strands (200) under a condition suitable to generate a plurality of library-splint complexes (300) each having a nick, or (2) hybridizing the plurality of DNA library molecules of step (j) to a plurality of double-stranded splint adaptors (600) under a condition suitable to generate a plurality of library-splint complexes (900) each having two nicks.

In some embodiments, the methods for appending an affinity tag to RNA molecules, further comprise step (l): contacting the library-splint complexes (300) or (900) with ligase enzyme under a condition to ligate the nicks and generate a plurality of covalently closed circular library molecules (400) or to generate a plurality of covalently closed circular library molecules (1000).

In some embodiments, the methods for appending an affinity tag to RNA molecules, further comprise step (m): conducting a rolling circle amplification reaction by contacting the plurality of covalently closed circular library molecules (400) or the plurality of covalently closed circular library molecules (1000) with strand displacing polymerase and a plurality of nucleotides (e.g., dATP, dCTP, dGTP, dTTP, and/or dUTP), under a condition to generate a plurality of concatemers. In some embodiments, the rolling circle amplification reaction can be conducted on-support or in-solution using the methods described herein. In some embodiments, the plurality of concatemers can be immobilized to a support and the concatemers can serve as template molecules for sequencing. In some embodiments, the sequencing can be conducted using any of the sequencing workflows described herein including two-stage sequencing workflow, sequencing-by-binding, or sequencing using labeled or non-labeled chain terminator nucleotides.

Supports with Low Non-Specific Binding Coatings

In some aspects, the present disclosure provides compositions and methods for use of a support having a plurality of surface primers immobilized thereon, for preparing any of the immobilized concatemers described herein. In some embodiments, the support is passivated with a low non-specific binding coating (e.g., FIG. 28). The surface coatings described herein may exhibit very low non-specific binding to reagents typically used for nucleic acid capture, amplification, and sequencing workflows, such as dyes, nucleotides, enzymes, and nucleic acid primers. The surface coatings may exhibit low background fluorescence signals or high contrast-to-noise (CNR) ratios compared to conventional surface coatings.

In some embodiments, the supports comprise a substrate (or support structure), one or more layers of a covalently or non-covalently attached low-binding, chemical modification layers, e.g., silane layers, polymer films, and one or more covalently or non-covalently attached primer sequences that may be used for tethering single-stranded target nucleic acid(s) to the support surface. In some embodiments, the formulation of the surface, e.g., the chemical composition of one or more layers, the coupling chemistry used to cross-link the one or more layers to the support surface and/or to each other, and the total number of layers, may be varied such that non-specific binding of proteins, nucleic acid molecules, and other hybridization and amplification reaction components to the support surface is minimized or reduced relative to a comparable monolayer. Often, the formulation of the surface may be varied such that non-specific hybridization on the support surface is minimized or reduced relative to a comparable monolayer. The formulation of the surface may be varied such that non-specific amplification on the support surface is minimized or reduced relative to a comparable monolayer. The formulation of the surface may be varied such that specific amplification rates and/or yields on the support surface are maximized. In some embodiments, amplification levels suitable for detection are achieved in no more than 2, 3, 4, 5, 6, 7, 8, 9, 10, 15, 20, 25, 30, or more than 30 amplification cycles in some cases disclosed herein.

The substrate or support structure that comprises the one or more chemically modified layers, e.g., layers of a low non-specific binding polymer, may be independent or alternatively may be integrated into another structure or assembly. For example, in some embodiments, the substrate or support structure may comprise one or more surfaces within an integrated or assembled microfluidic flow cell. The substrate or support structure may comprise one or more surfaces within a microplate format, e.g., the bottom surface of the wells in a microplate. As noted above, in some embodiments, the substrate or support structure comprises the interior surface (such as the lumen surface) of a capillary. In alternate embodiments, the substrate or support structure comprises the interior surface (such as the lumen surface) of a capillary etched into a planar chip.

In some embodiments, the attachment chemistry used to graft a first chemically modified layer to a surface will generally be dependent on both the material from which the surface is fabricated and the chemical nature of the layer. In some embodiments, the first layer may be covalently attached to the surface. In some embodiments, the first layer may be non-covalently attached, e.g., adsorbed to the surface through non-covalent interactions such as electrostatic interactions, hydrogen bonding, or van der Waals interactions between the surface and the molecular components of the first layer. In either case, the substrate surface may be treated prior to attachment or deposition of the first layer. Any of a variety of surface preparation techniques known to those of skill in the art may be used to clean or treat the surface. For example, and without limitation, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

In some embodiments, silane chemistries constitute one non-limiting approach for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding surfaces include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the surface, where the choice of components used may be varied to alter one or more properties of the surface, e.g., the surface density of functional groups and/or tethered oligonucleotide primers, the hydrophilicity/hydrophobicity of the surface, or the three three-dimensional nature (i.e., “thickness”) of the surface. Examples of polymers that may be used to create one or more layers of low non-specific binding material in any of the disclosed surfaces include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag-Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the substrate. Alternately, the surface coating may be patterned, such that the chemical modification layers are confined to one or more discrete regions of the substrate. For example, the surface may be patterned using photolithographic techniques to create an ordered array or random pattern of chemically modified regions on the surface. Alternately or in combination, the substrate surface may be patterned using, e.g., contact printing and/or ink-jet printing techniques. In some embodiments, an ordered array or random pattern of chemically modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In order to achieve low nonspecific binding surfaces, hydrophilic polymers may be nonspecifically adsorbed or covalently grafted to the surface. Typically, passivation is performed utilizing poly(ethylene glycol) (PEG, also known as polyethylene oxide (PEO) or polyoxyethylene) or other hydrophilic polymers with different molecular weights and end groups that are linked to a surface using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting surface. In some embodiments, oligonucleotide primers with different base sequences and base modifications (or other biomolecules, e.g., enzymes or antibodies) may be tethered to the resulting surface layer at various surface densities. In some embodiments, for example, both surface functional group density and oligonucleotide concentration may be varied to target a certain primer density range. Additionally, primer density can be controlled by diluting oligonucleotide with other molecules that carry the same functional group. For example, amine-labeled oligonucleotide can be diluted with amine-labeled polyethylene glycol in a reaction with an NHS-ester coated surface to reduce the final primer density. Primers with different lengths of linker between the hybridization region and the surface attachment functional group can also be applied to control surface density. Examples of suitable linkers include, but are not limited to, poly-T and poly-A strands at the 5′ end of the primer (e.g., 0 to 20 bases, e.g., 0, 1, 2, 3, 4, 5, 6, 7, 8, 9, 10, 11, 12, 13, 14, 15, 16, 17, 18, 19, 20 bases), PEG linkers (e.g., 3 to 20 monomer units, e.g., 3, 4, 5, 6, 7, 8, 9, 10, 11, 12, 13, 14, 15, 16, 17, 18, 19, 20 monomer units), and carbon-chain (e.g., C6, C12, C18, etc.). To measure the primer density, fluorescently labeled primers may be tethered to the surface and a fluorescence reading then compared with that for a dye solution of known concentration.

In order to scale primer surface density and add additional dimensionality to hydrophilic or amphoteric surfaces, surfaces comprising multi-layer coatings of PEG and other hydrophilic polymers have been developed. By using hydrophilic and amphoteric surface layering approaches that include, but are not limited to, the polymer/co-polymer materials described below, it is contemplated herein that it is possible to increase primer loading density on the surface significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

As noted, the low non-specific binding coatings of the present disclosure may exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, in some embodiments, exposure of the surface to fluorescent dyes (e.g., cyanine dyes such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g., polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g., polymerases) under a standardized set of conditions, followed by a specified rinse protocol and fluorescence imaging may be used as a quantitative tool for comparison of non-specific binding on supports comprising different surface formulations—provided that care has been taken to ensure that the fluorescence imaging is performed under a condition where fluorescence signal is linearly related (or related in a predictable manner) to the number of fluorophores on the support surface (e.g., under a condition where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein may exhibit a ratio of specific to nonspecific binding of a fluorophore, such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein may exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

As noted, in some embodiments, the degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed by detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or other molecules) per unit area. In some embodiments, the low-binding supports of the present disclosure may exhibit non-specific protein binding (or non-specific binding of other specified molecules, (e.g., cyanine dyes such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm².

For example, and without limitation, some modified surfaces disclosed herein exhibit nonspecific protein binding of less than 0.5 molecule/μm² following contact with a 1 μM solution of Cy3 labeled streptavidin (GE Amersham™) in phosphate buffered saline (PBS) buffer for 15 minutes and followed by 3 rinses with deionized water. In some embodiments, some modified surfaces disclosed herein exhibit nonspecific binding of Cy3 dye molecules of less than 0.25 molecules per μm². In some embodiments of independent nonspecific binding assays, 1 μM labeled Cy3 SA (ThermoFisher), 1 μM Cy5 SA dye (ThermoFisher), 10 μM Aminoallyl-dUTP-ATTO-647N (Jena Biosciences), 10 μM Aminoallyl-dUTP-ATTO-Rho11 (Jena Biosciences), 10 μM Aminoallyl-dUTP-ATTO-Rho11 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) are incubated on low binding substrates at 37° C., e.g., for 15 minutes, in a 384 well plate format. In certain embodiments, each well is rinsed 2-3× with 50 μL deionized RNase/DNase Free water and 2-3× with 25 mM ACES buffer at pH of about 7.4. The 384 well plates may then be imaged on a GE Typhoon instrument using the Cy3, AF555, or Cy5 filter sets (according to the dye test performed) and as specified by the manufacturer's instructions, at a PMT gain setting of 800 and resolution of 50-100 m. For higher resolution imaging, images may be collected, for example and without limitation, on an Olympus IX83 microscope (Olympus Corp., Center Valley, PA) with a total internal reflectance fluorescence (TIRF) objective lens (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. In some embodiments, dichroic mirrors may be purchased from Semrock (IDEX Health & Science, LLC, Rochester, New York), e.g., 405, 488, 532, or 633 nm dichroic reflectors/beamsplitters, and band pass filters chosen as 532 LP or 645 LP concordant with the appropriate excitation wavelength. In some embodiments, some modified surfaces disclosed herein exhibit nonspecific binding of dye molecules of less than 0.25 molecules per μm².

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, in some embodiments, adequate wash steps may be performed in less than 30 seconds.

The low-binding surfaces of the present disclosure may exhibit significant improvement in stability or durability to prolonged exposure to solvents and elevated temperatures, or to repeated cycles of solvent exposure or changes in temperature. For example, in some embodiments, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100-fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100-fold greater than a signal of an adjacent amplified nucleic acid population region of the surface.

In some embodiments, fluorescence images of the disclosed low background surfaces when used in nucleic acid hybridization or amplification applications to create clusters of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer (e.g., capture primers) may be attached or tethered to the support surface. In some embodiments, the one or more types of adapters or primers may comprise spacer sequences, adapter sequences for hybridization to adapter-ligated target library nucleic acid sequences, forward amplification primers, reverse amplification primers, sequencing primers, and/or molecular barcoding sequences, or any combination thereof. In some embodiments, 1 primer or adapter sequence may be tethered to at least one layer of the surface. In some embodiments, at least 2, 3, 4, 5, 6, 7, 8, 9, 10, or more than 10 different primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers on the low binding support surfaces of the present disclosure may range from about 100 primer molecules per μm² to about 100,000 primer molecules per μm². In some embodiments, the resultant surface density of primers on the low binding support surfaces of the present disclosure may range from about 100,000 primer molecules per μm² to about 10¹⁵ primer molecules per μm². In some embodiments, the surface density of primers may be at least 1,000, at least 10,000, at least 100,000, or at least 10¹⁵ primer molecules per μm². In some embodiments, the surface density of primers may be at most 10,000, at most 100,000, at most 1,000,000, or at most 10¹⁵ primer molecules per μm². Any of the lower and upper values described in this paragraph may be combined to form a range included within the present disclosure, for example, in some embodiments the surface density of primers may range from about 10,000 molecules per μm² to about 10¹⁵ molecules per μm². Those of skill in the art will recognize that the surface density of primer molecules may have any value within this range, e.g., about 455,000 molecules per μm². In some embodiments, the surface density of target library nucleic acid sequences initially hybridized to adapter or primer sequences on the support surface may be less than or equal to that indicated for the surface density of tethered primers. In some embodiments, the surface density of clonally amplified target library nucleic acid sequences hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000 per μm², while also comprising at least a second region having a substantially different local density.

The low non-specific binding coating may comprise one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule to about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments, at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some embodiments, an organic component of the solvent mixture used may comprise at least 1%, 5%10%, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

Fluorescence imaging may be performed using any of a variety of fluorophores, fluorescence imaging techniques, and fluorescence imaging instruments known to those of skill in the art. Examples of suitable fluorescence dyes that may be used (e.g., by conjugation to nucleotides, oligonucleotides, or proteins) include, but are not limited to, fluorescein, rhodamine, coumarin, cyanine, and derivatives thereof, including the cyanine derivatives Cyanine dye-3 (Cy3), Cyanine dye-5 (Cy5), Cyanine dye-7 (Cy7), etc. Examples of fluorescence imaging techniques that may be used include, but are not limited to, fluorescence microscopy imaging, fluorescence confocal imaging, two-photon fluorescence, and the like. Examples of fluorescence imaging instruments that may be used include, but are not limited to, fluorescence microscopes equipped with an image sensor or camera, confocal fluorescence microscopes, two-photon fluorescence microscopes, or custom instruments that comprise a suitable selection of light sources, lenses, mirrors, prisms, dichroic reflectors, apertures, and image sensors or cameras, etc. A non-limiting example of a fluorescence microscope equipped for acquiring images of the disclosed low-binding support surfaces and clonally-amplified colonies (polonies) of template nucleic acid sequences hybridized thereon is the Olympus IX83 inverted fluorescence microscope equipped with) 20×, 0.75 NA, a 532 nm light source, a bandpass and dichroic mirror filter set optimized for 532 nm long-pass excitation and Cy3 fluorescence emission filter, a Semrock 532 nm dichroic reflector, and a camera (Andor sCMOS, Zyla 4.2) where the excitation light intensity is adjusted to avoid signal saturation. Often, the support surface may be immersed in a buffer (e.g., 25 mM ACES, pH 7.4 buffer) while the image is acquired.

In some instances, the performance of nucleic acid hybridization and/or amplification reactions using the disclosed reaction formulations and low non-specific binding supports may be assessed using fluorescence imaging techniques, where the contrast-to-noise ratio (CNR) of the images provides a key metric in assessing amplification specificity and non-specific binding on the support. CNR is commonly defined as: CNR=(Signal−Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized). The surfaces of the instant disclosure are also provided in International Application Serial No. PCT/US2019/061556, which is hereby incorporated by reference in its entirety.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) may exist within the region occupied by an amplified DNA colony. In some embodiments, the combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal may arise from a variety of sources; a non-limiting few examples include auto-fluorescence from consumable flow cells, non-specific adsorption of detection molecules that yield spurious fluorescence signals that may obscure the signal from the ROI, or the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (S)—Bᵢₙₜₑᵣ in the FOV) yields a discernable feature that can be classified. In some instances, the intrastitial background (Bᵢₙₜᵣₐ) can contribute a confounding fluorescence signal that is not specific to the target of interest but is present in the same ROI thus making it far more difficult to average and subtract.

In some embodiments, the implementation of nucleic acid amplification on the low-binding substrates of the present disclosure may decrease the Bᵢₙₜₑᵣ background signal by reducing non-specific binding, may lead to improvements in specific nucleic acid amplification, and may lead to a decrease in non-specific amplification that can impact the background signal arising from both the interstitial and intrastitial regions. In some instances, the disclosed low-binding support surfaces, optionally used in combination with the disclosed hybridization buffer formulations, may lead to improvements in CNR by a factor of 2, 5, 10, 100, or 1000-fold over those achieved using conventional supports and hybridization, amplification, and/or sequencing protocols. Although described here in the context of using fluorescence imaging as the read-out or detection mode, the same principles generally apply to the use of the disclosed low non-specific binding supports and nucleic acid hybridization and amplification formulations for other detection modes as well, including both optical and non-optical detection modes.

In some embodiments, the disclosed low-binding supports, optionally used in combination with the disclosed hybridization and/or amplification protocols, yield solid-phase reactions that exhibit: (i) negligible non-specific binding of protein and other reaction components (thus minimizing substrate background), (ii) negligible non-specific nucleic acid amplification product, and (iii) provide tunable nucleic acid amplification reactions.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

In some embodiments, a fluorescence image of the surface exhibits a contrast-to-noise ratio (CNR) of at least 20 when a sample nucleic acid molecule or complementary sequences thereof are labeled with a Cyanine dye-3 (Cy3) fluorophore, and when the fluorescence image is acquired using an inverted fluorescence microscope (e.g., Olympus IX83) with a 20×0.75 NA objective, a 532 nm light source, a bandpass and dichroic mirror filter set optimized for 532 nm excitation and Cy3 fluorescence emission, and a camera (e.g., Andor sCMOS, Zyla 4.2) under non-signal saturating conditions while the surface is immersed in a buffer (e.g., 25 mM ACES, pH 7.4 buffer).

### ENUMERATED EMBODIMENTS

Provided below are enumerated paragraphs describing specific embodiments of the present disclosure:
1. A method for sequencing a nucleic acid molecule, the method comprising:
(a) providing a plurality of clonal nucleic acid molecules each having the same barcode sequence attached in proximity to a first end;
(b) for each nucleic acid molecule, fragmenting the nucleic acid molecule adjacent to a random portion of the nucleic acid molecule to provide a second end;
(c) for each nucleic acid molecule, joining the first end with the second end to provide a circularized nucleic acid molecule having the barcode sequence adjacent to the random portion of the nucleic acid sequence;
(d) for each nucleic acid molecule, sequencing the barcode and the random portion of the nucleic acid molecule; and
(e) assembling the sequence of the nucleic acid molecule from the plurality of random portions of the nucleic acid molecule.
2. The method of embodiment 1, wherein the method is performed with a plurality of clonal nucleic acid populations each having a different barcode sequence attached thereto, and a separate sequence is assembled in (e) for each of the barcode sequences.
3. A method comprising:
(a) providing a plurality of target nucleic acid molecules;
(b) providing a plurality of adapter fragments, each comprising a first region that is identical for each of the adapter fragments and a second region that is unique for each of the adapter fragments;
(c) attaching the adapter fragments of (b) to the target nucleic acid molecules of (a) to create a plurality of adapter-ligated target molecules;
(d) amplifying the adapter-ligated target molecules of (c);
(e) fragmenting the amplified molecules of (d);
(f) circularizing the fragmented molecules of (e);
(g) fragmenting the circularized molecules of (f); and
(h) sequencing the fragmented molecules of (g).
4. A method comprising:
(a) providing a plurality of target nucleic acid molecules;
(b) providing a plurality of adapter fragments, each comprising a first region that is identical for each of the adapter fragments and a second region that is unique for each of the adapter fragments;
(c) attaching the adapter fragments of (b) to the target nucleic acid molecules of (a) to create a plurality of adapter-ligated target molecules;
(d) amplifying the adapter-ligated target molecules of (c);
(e) fragmenting the amplified molecules of (d);
(f) circularizing the fragmented molecules of (e); and
(g) sequencing the circularized molecules of (f).
5. The method of embodiment 3 or embodiment 4, wherein the attaching in (c) is performed by PCR.
6. The method of embodiment 3 or embodiment 4, wherein the attaching in (c) is performed by ligation.
7. A method for obtaining nucleic acid sequence information from a nucleic acid molecule comprising a target nucleotide sequence by assembling a series of nucleic acid sequences into a longer nucleic acid sequence, said method comprising:
(a) attaching a first adapter comprising an outer polymerase chain reaction (PCR) primer region or nucleic acid amplification region, an inner sequencing primer region, and a central barcode region to each end of a plurality of linear nucleic acid molecules to form barcode-tagged molecules;
(b) replicating the barcode-tagged molecules to obtain a library of barcode-tagged molecules;
(c) breaking the barcode-tagged molecules, thereby generating linear, barcode-tagged fragments comprising the barcode region at one end and a region of unknown sequence at the other end;
(d) circularizing the linear, barcode-tagged fragments comprising the barcode region at one end and a region of unknown sequence from an interior portion of the target nucleotide sequence at the other end, thereby bringing the barcode region into proximity with the region of unknown sequence;
(e) fragmenting the circularized, barcode-tagged fragments into linear, barcode-tagged fragments;
(f) attaching a second adapter to each end of the linear, barcode-tagged fragments to form double adapter-ligated barcode-tagged nucleic acid fragments;
(g) replicating all or part of the double adapter-ligated barcode-tagged nucleic acid fragments;
(h) sequencing the double adapter-ligated barcode-tagged nucleic acid fragments;
(i) sorting a series of sequenced nucleic acid fragments into independent groups; and assembling each group of reads into a longer nucleic acid sequence.
8. The method of embodiment 7, further comprising fragmenting the nucleic acid molecule comprising the target nucleotide sequence into a plurality of linear nucleic acid sequences prior to attaching the first adapter.
9. The method of embodiment 7 or 8, wherein the first adapter attached at the 5′ end comprises a different barcode than the first adapter attached at the 3′ end.
10. The method of embodiment 7 or 8, wherein the first adapter attached at the 5′ end and the first adapter attached at the 3′ end comprises the same barcode.
11. The method of any one of embodiments 7 to 10, wherein replicating the barcode-tagged sequences is carried out by PCR.
12. The method of any one of embodiments 7 to 11, wherein replicating the barcode-tagged sequences to obtain a library of barcode-tagged sequences is carried out using a primer complementary to the PCR primer region.
13. The method of any one of embodiments 7 to 12, further comprising removing the PCR primer region from the barcode-tagged sequences.
14. The method of embodiment 13, wherein the removing the PCR primer region is carried out before circularizing the barcode-tagged fragments.
15. The method of any one of embodiments 7 to 14, wherein breaking the barcode-tagged sequences is carried out by an enzyme.
16. The method of any one of embodiments 7 to 15, wherein the breaking is carried out at random locations on the nucleic acid sequences.
17. The method of any one of embodiments 7 to 16, wherein the second adapter comprises two nucleic acid strands of different lengths, wherein the strand attached at the 5′ ends of a linear, barcode-tagged fragment is of a different length than the strand attached at the 3′ ends of a linear, barcode-tagged fragment, wherein one end of the second adapter is double stranded to facilitate ligation and the other end of the second adapter comprises a 3′ single-stranded overhang, and wherein only the longer of the two oligonucleotides comprises a sequence complementary to a second sequencing primer and comprises sufficient length to allow annealing of that primer.
18. The method of any one of embodiments 7 to 17, wherein replicating the double adapter-ligated barcode-tagged nucleic acid fragments is carried out using two primers, the first of which is complementary to a constant sequence from the barcode-containing adapter, and the second of which is complementary to the overhanging sequence of the asymmetric adapter, and which together add sequences necessary for nucleic acid sequencing.
19. The method of embodiment 18, wherein the replicating is carried out using PCR.
20. The method of any one of embodiments 7 to 19, wherein sequencing the double adapter-ligated barcode-tagged nucleotide fragments is carried out beginning with the barcode region followed by the target sequence.
21. The method of any one of embodiments 7 to 20, wherein sorting the series of sequenced nucleic acid fragments into independent groups is based on shared barcodes.
22. The method of any one of embodiments 7 to 15, wherein assembling each group is carried out independent of all other groups.
23. The method of any one of embodiments 7 to 21, further comprising selecting the plurality of linear nucleic acid sequences on the basis of size prior to attaching the first adapter.
24. The method of any one of embodiments 7 to 22, further comprising selecting the fragments on the basis of size prior to sequencing.
25. The method of any one of embodiments 7 to 23, wherein the enzyme that generates linear, tagged nucleotide fragments is a double-stranded DNA fragmentase.
26. The method of embodiment 13, wherein the PCR primer region is removed by an enzyme that excises uracils and breaks the phosphate backbone.
27. The method of embodiment 13, wherein the PCR primer region comprises methylated nucleotides and the PCR primer region is removed by restriction enzymes specific for methylated sequences.
28. The method of any one of embodiments 7 to 27, wherein nucleic acid sequence information is obtained for a longer nucleic acid sequence comprising a length of at least about 500 bases.
29. The method of any one of embodiments 7 to 28, wherein nucleic acid sequence information is obtained for a longer nucleic acid sequence comprising a length of at least about 1000 bases.
30. The method of any one of embodiments 7 to 29, wherein nucleic acid sequence information is obtained for a longer nucleic acid sequence comprising a length of at least 1000 or more bases.
31. The method of any one of embodiments 7 to 30, wherein nucleic acid sequence information is obtained for a longer nucleic acid sequence comprising a length from about 1 kilobase to about 20 kilobases.
32. The method of any one of embodiments 7 to 31, wherein nucleic acid sequence information is obtained for a longer nucleic acid sequence comprising a length of up to about 12 kilobases.
33. The method of any one of embodiments 7 to 32, wherein the nucleic acid sequence information comprises greater than about 95% fidelity to the target nucleotide sequence.
34. The method of any one of embodiments 7 to 33, wherein the target nucleotide sequence originates from genomic DNA.
35. The method of any one of embodiments 7 to 34, wherein the nucleic acid sequence information is obtained in less than three days.
36. The method of any one of embodiments 7 to 35, wherein (a)-(j) are carried out in one tube.
37. A method comprising:
(a) sequencing a plurality of nucleic acids located at positions on an array; and measuring a phenotype of a molecule at the positions on the array.
38. A method comprising sequencing the genetic component of the members of a polypeptide display library.
39. A method for generating a plurality of linked sequence-phenotype pairs, the method comprising:
(a) applying to an array, a library of mutant proteins associated with their encoding nucleic acid, wherein the library is applied with essentially one mutant per array position;
(b) measuring the phenotype of the protein at each array position; and
(c) sequencing at least part of the nucleic acid associated with the protein at each array position, thereby generating a linked sequence-phenotype pair at each array position.
40. A method for generating a plurality of linked sequence-phenotype pairs, the method comprising:
(a) applying to an array, a library of mutant nucleic acids, wherein the library is applied with essentially one mutant per array position;
(b) measuring the phenotype of the nucleic acid at each array position; and
(c) sequencing at least part of the nucleic acid at each array position, thereby generating a linked sequence-phenotype pair at each array position.
41. A method for generating a plurality of linked sequence-phenotype pairs, the method comprising:
(a) applying to an array, a library of mutant nucleic acids, wherein the library is applied with essentially one mutant per array position;
(b) expressing the proteins encoded by the nucleic acids on the array; and
(c) measuring the phenotype of the proteins at each array position; and
(d) sequencing at least part of the nucleic acid at each array position, thereby generating a linked sequence-phenotype pair at each array position.
42. A method for generating a plurality of linked sequence-phenotype pairs, the method comprising:
(a) synthesizing a plurality of nucleic acids at fixed positions on an array;
(b) expressing the proteins encoded by the nucleic acids on the array; and
(c) measuring the phenotype of the protein at each array position, thereby generating a linked sequence-phenotype pair at each array position.
43. A method for generating a plurality of linked sequence-phenotype pairs, the method comprising:
(a) applying to an array of immobilized nucleic acids, a library of mutant proteins associated with their encoding nucleic acid, wherein the immobilized nucleic acids hybridize with the nucleic acids that are associated with the mutant proteins; and
(b) measuring the phenotype of the protein at each array position, thereby generating a linked sequence-phenotype pair at each array position.
44. The method of any of the previous embodiments, further comprising analyzing the linked sequence-phenotype pairs to determine:
   (i) a sequence that expresses or has a high probability of expressing a protein having a desired phenotype; and/or
   (ii) a plurality of sequences, wherein at least one of the sequences has a high probability of expressing a protein having a desired phenotype; and/or
   (iii) the effect of individual sequence mutations on the phenotype of the protein expressed from the sequence; and/or
   (iv) the effect of a group of sequence mutations on the phenotype of the protein expressed from the sequence; and/or
   (v) a set of allowed mutations at a sequence position, wherein the protein expressed from the sequence has an acceptable phenotype.
45. The method of any of the previous embodiments, further comprising analyzing the linked sequence-phenotype pairs to determine:
   (1) a nucleic acid molecule that has a high probability of having a desired phenotype; and/or
   (2) a plurality of nucleic acid molecules, wherein at least one of the molecules that has a high probability of having a desired phenotype; and/or
   (3) the effect of individual sequence mutations on the phenotype of a nucleic acid molecule; and/or
   (4) the effect of a group of sequence mutations on the phenotype of a nucleic acid molecule; and/or
   (5) a set of allowed mutations at a sequence position, wherein the nucleic acid molecule has an acceptable phenotype.
46. Use of the method of any of the previous embodiments to evolve a protein to a desired phenotype.
47. A method of directed evolution, the method comprising:
(a) from a first plurality of sequences, generating a first plurality of linked sequence-phenotype pairs according to the methods of any of the embodiments;
(b) analyzing the first linked sequence-phenotype pairs to design a plurality of second sequences, wherein at least one of the second sequences has a high probability of expressing a protein having a desired phenotype;
(c) optionally generating and analyzing a second plurality of linked sequence-phenotype pairs according to the methods of any of the embodiments; and
(d) optionally iterating this cycle as many times as necessary to isolate a protein with the desired phenotype.
48. A method of directed evolution, the method comprising:
(a) generating a library of mutant polypeptides associated with their encoding nucleic acids;
(b) applying the library to an array, whereby there is essentially one mutant per array position;
(c) measuring the phenotype of the mutant polypeptide at each array position
(d) sequencing at least part of the nucleic acid at each array position; and
(e) analyzing the linked phenotype data and sequence data, wherein the linked data informs mutations suitable for evolving the polypeptide toward a desired phenotype.
49. An apparatus comprising an array, wherein the array is capable of sequencing nucleic acids and measuring the phenotype of a protein.
50. An apparatus comprising a member that collects linked sequence-phenotype data from an array of nucleic acid-protein pairs.
51. The method or apparatus of any of the previous embodiments, wherein the array comprises at least 10⁴ positions.
52. The method or apparatus of any of the previous embodiments, wherein the array comprises at least 10⁵ positions.
53. The method or apparatus of any of the previous embodiments, wherein the array comprises at least 10⁶ positions.
54. The method or apparatus of any of the previous embodiments, wherein the array comprises at least 10⁷ positions.
55. The method or apparatus of any of the previous embodiments, wherein the array comprises at least 10′ positions.
56. The method or apparatus of any of the previous embodiments, wherein the array comprises one or more sensors.
57. The method or apparatus of any of the previous embodiments, wherein the array is interrogated by one or more sensors.
58. The method or apparatus of any of the previous embodiments, wherein the one or more sensors comprise a chemFET sensor.
59. The method or apparatus of any of the previous embodiments, wherein the one or more sensors measure a signal associated with fluorescence, pH change, luminescence, or any combination thereof.
60. The method or apparatus of any of the previous embodiments, wherein the signal is proportional to a phenotype or relatable to a phenotype by a calibration curve.
61. The method or apparatus of any of the previous embodiments, wherein the signal is a change in temperature at the array position.
62. The method of any of the previous embodiments, wherein the mutant proteins are associated with their encoding nucleic acid by attachment to a microbead.
63. The method of any of the previous embodiments, wherein the mutant proteins are associated with their encoding nucleic acid by ribosome display.
64. The method of any of the previous embodiments, wherein the mutant proteins are associated with their encoding nucleic acid by RNA display.
65. The method of any of the previous embodiments, wherein the mutant proteins are associated with their encoding nucleic acid by DNA display.
66. The method or apparatus of any of the previous embodiments, wherein the phenotype is enzyme rate.
67. The method or apparatus of any of the previous embodiments, wherein the phenotype is enzyme specificity.
68. The method or apparatus of any of the previous embodiments, wherein the phenotype is binding affinity.
69. The method or apparatus of any of the previous embodiments, wherein the phenotype is binding specificity.
70. The method or apparatus of any of the previous embodiments, further comprising contacting the proteins to a plurality of solutions comprising substrates at a plurality of concentrations.
71. The method or apparatus of any of the previous embodiments, further comprising contacting the proteins to a plurality of solutions comprising ligands at a plurality of concentrations.
72. The method or apparatus of any of the previous embodiments, further comprising measuring the phenotype at a plurality of temperatures.
73. The method or apparatus of any of the previous embodiments, wherein the phenotype is stability when exposed to a chemical condition or a temperature.
74. The method of any of the previous embodiments, wherein the protein is expressed using cell-free protein synthesis.
75. The method of any of the previous embodiments, wherein the protein is expressed in an emulsion.
76. The method of any of the previous embodiments, wherein the nucleic acid is amplified in an emulsion PCR.
77. The method of any of the previous embodiments, wherein the protein is labeled at a defined stoichiometry, wherein the label is used to determine the number of proteins at the array position.
78. The method of any of the previous embodiments, wherein the protein associates with a known stoichiometry of probe molecule on the array.
79. The method of any of the previous embodiments, wherein the probe molecule is an antibody linked to a fluorescent molecule, an enzyme, or an enzymatic substrate.
80. The method of any of the previous embodiments, wherein the nucleic acid is sequenced more than once.
81. The method of any of the previous embodiments, wherein the nucleic acid is sequenced a plurality of times starting from various positions along the nucleic acid sequence.
82. The method of any of the previous embodiments, wherein the nucleic acid is amplified in an emulsion PCR, wherein a plurality of secondary nucleic acid molecules are created corresponding to different portions of the nucleic acid, wherein the secondary nucleic acid molecules are sequenced.
83. The method of embodiment 7, wherein the double adaptor-ligated barcode-tagged nucleic acid fragments comprise a plurality of library molecules (100) each comprising: (i) a surface pinning primer binding site (120), (ii) a left sample index sequence (160), (iii) a forward sequencing primer binding site (140), (iv) a left UMI sequence (180), (v) an insert sequence (e.g., sequence of interest) (110), (vi) a reverse sequencing primer binding site (150), (vii) a right sample index sequence (170) which optionally includes a 3-mer random sequence, and (viii) a surface capture primer binding site (130).
84. The method of embodiment 83, further comprising: generating single stranded library molecules from the plurality of library molecules (100).
85. The method of embodiment 84, further comprising: forming a plurality of library-splint complexes (300) comprising:
a) providing a plurality of single-stranded nucleic acid library molecules (100) each comprising: (i) a surface pinning primer binding site (120), (ii) a left sample index sequence (160), (iii) a forward sequencing primer binding site (140), (iv) a left UMI sequence (180), (v) an insert sequence (e.g., sequence of interest) (110), (vi) a reverse sequencing primer binding site (150), (vii) a right sample index sequence (170) which optionally includes a 3-mer random sequence, and (viii) a surface capture primer binding site (130);
b) providing a plurality of single-stranded splint strands (200) wherein individual single-stranded splint strands (200) in the plurality comprise a first region (210) that is capable of hybridizing with the at least a first left universal adaptor sequence (120) of an individual library molecule, and a second region (220) that is capable of hybridizing with the at least a first right universal adaptor sequence (130) of the individual library molecule;
c) hybridizing the plurality of single-stranded splint strands (200) with plurality of single-stranded nucleic acid library molecules (100) such that the first region of one of the single-stranded splint strands (210) anneals to the at least first left universal adaptor sequence (120) of the library molecule, and such that the second region of the single-stranded splint strand (220) anneals to the at least first right universal sequence (130) of the library molecule, thereby circularizing individual library molecules to form a plurality of library-splint complexes (300) having a nick between the terminal 5′ and 3′ ends of the library molecule, wherein the nick is enzymatically ligatable; and
d) ligating the nick in the plurality of library-splint complexes (300) thereby generating a plurality of covalently closed circular library molecules (400).
86. The method of embodiment 85, further comprising: (e) distributing the plurality of covalently closed circular library molecules (400) onto a support having a plurality of surface primers immobilized on the support, under a condition suitable for hybridizing individual covalently closed circular library molecules (400) to individual immobilized surface primers thereby immobilizing the plurality of covalently closed circular library molecules (400).
87. The method of embodiment 86, further comprising: (f) contacting the plurality of immobilized covalently closed circular library molecules (400) with a plurality of strand-displacing polymerases and a plurality of nucleotides, under a condition suitable to conduct a rolling circle amplification reaction on the support using the plurality of surface primers as immobilized amplification primers and the plurality of covalently closed circular library molecules (400) as template molecules, thereby generating a plurality of immobilized nucleic acid concatemer molecules.
88. The method of embodiment 87, further comprising: sequencing the plurality of immobilized nucleic acid concatemer molecules.
89. The method of embodiment 88, wherein the sequencing comprises:
a) contacting the plurality of immobilized concatemer molecules with (i) a plurality of sequencing polymerases and (ii) a plurality of the soluble sequencing primers, wherein the contacting is conducted under a condition suitable to form a plurality of complexed polymerases each comprising a sequencing polymerase bound to a nucleic acid duplex wherein the nucleic acid duplex comprises a concatemer molecule hybridized to a soluble sequencing primer; b) contacting the plurality of complexed sequencing polymerases with a plurality of nucleotides under a condition suitable for binding at least one nucleotide to a complexed sequencing polymerase, wherein the plurality of nucleotides comprises at least one nucleotide analog labeled with a fluorophore and having a removable chain terminating moiety at the sugar 3′ position;
c) incorporating at least one nucleotide into the 3′ end of the hybridized sequencing primers thereby generating a plurality of nascent extended sequencing primers; and
d) detecting the incorporated nucleotide and identifying the nucleo-base of the incorporated nucleotide.
90. The method of embodiment 88, wherein the sequencing comprises:
a) contacting the plurality of immobilized concatemer molecules with (i) a plurality of sequencing polymerases and (ii) a plurality of the soluble sequencing primers, wherein the contacting is conducted under a condition suitable to form a plurality of first complexed polymerases each comprising a sequencing polymerase bound to a nucleic acid duplex wherein the nucleic acid duplex comprises a concatemer molecule hybridized to a soluble sequencing primer;
b) contacting the plurality of complexed sequencing polymerases with a plurality of detectably labeled multivalent molecules to form a plurality of multivalent-complexed polymerases, under a condition suitable for binding complementary nucleotide units of the multivalent molecules to at least two of the plurality of first complexed polymerases thereby forming a plurality of multivalent-complexed polymerases, and the condition inhibits incorporation of the complementary nucleotide units into the sequencing primers of the plurality of multivalent-complexed polymerases, wherein individual multivalent molecules in the plurality of multivalent molecules comprise a core attached to multiple nucleotide arms and each nucleotide arm is attached to a nucleotide unit;
c) detecting the plurality of multivalent-complexed polymerases; and
d) identifying the nucleo-base of the complementary nucleotide units that are bound to the plurality of first complexed polymerases in the plurality of multivalent-complexed polymerases, thereby determining the sequence of the nucleic acid template.
91. The method of embodiment 90, further comprising:
e) dissociating the plurality of multivalent-complexed polymerases and removing the plurality of first sequencing polymerases and their bound multivalent molecules, and retaining the plurality of nucleic acid duplexes;
f) contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a retained nucleic acid duplex;
g) contacting the plurality of second complexed polymerases with a plurality of non-labeled nucleotides, wherein the contacting is conducted under a condition suitable for binding complementary nucleotides from the plurality of nucleotides to at least two of the second complexed polymerases of step (f) thereby forming a plurality of nucleotide-complexed polymerases and the condition is suitable for promoting incorporation of the bound complementary nucleotides into the sequencing primers of the nucleotide-complexed polymerases.
92. A method for forming at least one avidity complex, comprising:
a) binding a first universal nucleic acid primer, a first DNA polymerase, and a first multivalent molecule to a first portion of the concatemer molecules of embodiment 90, thereby forming a first binding complex, wherein a first nucleotide unit of the first multivalent molecule binds to the first DNA polymerase; and
b) binding a second universal nucleic acid primer, a second DNA polymerase, and the first multivalent molecule to a second portion of the same concatemer template molecule thereby forming a second binding complex, wherein a second nucleotide unit of the first multivalent molecule binds to the second DNA polymerase, wherein the first and second binding complexes which include the same multivalent molecule forms an avidity complex, wherein the first multivalent molecule comprises a core attached to multiple nucleotide arms and each nucleotide arm is attached to a nucleotide unit, and wherein the concatemer molecule comprises two or more tandem repeat sequences of a sequence of interest (110) and a universal primer binding site that binds the first and second universal nucleic acid primers.
93. A method for sequencing by forming at least one avidity complex, comprising:
a) binding a first universal nucleic acid primer, a first DNA polymerase, and a first multivalent molecule to a first portion of the concatemer molecules of embodiment 90, thereby forming a first binding complex, wherein a first nucleotide unit of the first multivalent molecule binds to the first DNA polymerase;
b) binding a second universal nucleic acid primer, a second DNA polymerase, and the first multivalent molecule to a second portion of the same concatemer template molecule thereby forming a second binding complex, wherein a second nucleotide unit of the first multivalent molecule binds to the second DNA polymerase, wherein the first and second binding complexes which include the same multivalent molecule forms an avidity complex, wherein the first multivalent molecule comprises a core attached to multiple nucleotide arms and each nucleotide arm is attached to a nucleotide unit, wherein the concatemer molecule comprises two or more tandem repeat sequences of a sequence of interest (110) and a universal primer binding site that binds the first and second universal nucleic acid primers, and wherein the contacting is conducted under a condition suitable to inhibit polymerase-catalyzed incorporation of the bound first and second nucleotide units in the first and second binding complexes;
c) detecting the first and second binding complexes on the same concatemer template molecule, and identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

### Examples

Additional aspects and details of the invention will be apparent from the following examples, which are intended to be illustrative rather than limiting.

### Example 1—Standard Protocols

Standard protocols used in the instant Examples of the disclosure are provided infra. A solution of two oligonucleotides (e.g., where the first (the barcode-containing oligo) was any of oligo 1, oligo 2, oligo 3, or oligo 4, and the second (the extension oligo) was any of oligo 5, oligo 6, or oligo 7, where oligo 5 is used with oligo 1 or oligo 4; oligo 6 is used with oligo 1, oligo 2, or oligo 4; and oligo 7 is used with oligo 3—the various oligos corresponding to those shown in Table 1 below), at 2 μM and 5 μM, respectively in NEBuffer 2 (New England Biolabs™ (NEB), Ipswich, MA) was heated to 95° C. for 10 minutes and allowed to cool to 37° C. over a timeframe of 30 minutes. Five units of Klenow exo-(NEB) and 0.3 mM each dNTP (NEB) was added and the mixture was incubated at 37° C. for 60 minutes.

The library DNA to be sequenced was linearized and fragmented to the desired size by restriction digestion, fragmentation, or PCR as necessary. Depending on the source of the nucleic acid and the goals of the project, the nucleic acid was fragmented into sizes from about 1 kb to about 20 kb. For example, genomic DNA is usually sheared to about 10 kb; in other examples, genes of about 3 kb comprise the sequence of interest. The gene can be amplified from source DNA or cut out of a larger genome with restriction enzymes using standard techniques. The DNA to be sequenced was typically diluted to 50 μL at 10 ng/L and fragmented into approximately 10 kb pieces with a g-TUBE (Covaris, Woburn, MA) by centrifugation at 4,200 g according to the manufacturer's protocol.

The DNA was end-repaired with the NEBNext™ End Repair Module (NEB) according to the manufacturer's suggested protocol and purified with a Zymo DNA Clean & Concentrator column (Zymo Research™, Irvine, CA) and eluted in 20 μL of buffer EB (an elution buffer used in eluting DNA). The DNA was then dT-tailed by incubation in 1×NEB buffer 2 with 1 mM dTTP (Life Technologies™, Grand Island, NY), 5 units Klenow exo-, and 10 units polynucleotide kinase at 37° C. for 1 hour.

250 fmol of library DNA and 5 pmol of barcoded tripartite adapters comprising an outer PCR primer region, an inner sequencing primer region, and a central barcode region were ligated with TA/Blunt MasterMix (NEB) according to the manufacturer's protocol, purified with a Zymo column or with gel purification with size selection with the Qiagen® Gel Extraction kit and eluted in 20 μL of buffer EB. The tripartite adapters, see, e.g., oligo 1 in Table 1, were designed so that barcode number takes into consideration target number. For example, an adapter comprising a 16N barcode worked for about 10 to about 20 million target sequences.

Two single-stranded oligonucleotides were ordered from a supplier, annealed together, and the shorter one extended to form the double-stranded adapter. The number of possible barcode sequences is 4ⁿ, where n is the number of degenerate bases. That number should be at least 100 times higher than the number of DNA molecules to be tagged to ensure that each molecule receives two unique tags. For example, n=16 has been used in experiments described herein (4¹⁶=4.3 billion). In various aspects, the barcode is made shorter (to maximize the portion of the sequencing read that reads target sequence) or longer (to ensure that no two molecules get identical barcodes).

Oligo 5, oligo 6 and oligo 7, shown in Table 1 below, represent both the shorter adapter extension oligo described herein above and the PCR primer (see Rungpragayphan et al., J. Mol. Biol. 318:395-405, 2002). Theoretically, the extension oligo may be any sequence long enough for primer annealing during PCR. The extension oligo annealed to the barcode-containing oligo and was extended by Klenow exo-polymerase, copying the barcode and forming a dA-tailed double-stranded adapter. The region on the 5′ end of the barcode-containing oligo was the sequence from the Illumina Universal sequencing primer. If a different sequencing primer was used for sequencing, the barcode-containing oligo should be modified accordingly.

The adapters were ligated at both ends of the DNA. A single adapter is ligated to each end of the nucleic acid by including an overhang on the 3′strand of the non-ligating end, thus blocking concatemerization on the end of the adapter. Library molecules that failed to ligate to an adapter at both ends were removed by incubation with 10 units of exonuclease III (NEB) and 20 units of exonuclease I (NEB) in NEBuffer 1 for 45 minutes at 37° C., followed by 20 minutes at 80° C.

Oligo 2, shown in Table 1 below, comprises an example of one strand of the tripartite adapter. The oligo, from 5′ to 3′, comprises: (1) NNN, which is an optional degenerate 5′ end to reduce sequence bias of ligation, (2) CCTACACGACGCTCTTCCGATCT (SEQ ID NO:55), which is the annealing sequence for oligo 11 (shown in Table 1 below), which adds the Illumina TruSeq Universal adapter during the final limited-cycle PCR; (3) NNNNNNNNNNNNNNNN, which is the degenerate barcode sequence; (4) CC, which is a short defined sequence to confirm that the previous bases comprise the barcode and to promote biotin-dCTP incorporation during end repair; (5) AGGAATAGTTATGTGCATTAATGAATGG (SEQ ID NO:54), which is an annealing sequence for oligo 6 (shown in Table 1 below), which both extends oligo 2 (shown in Table 1 below) to form the double-stranded tripartite adapter and is the primer for the first PCR; and (6) CGCC, which is a short overhanging sequence to prevent ligation on this end of the tripartite adapter, and which can be extended to include a primer annealing site for linear amplification.

The ligation product was quantified with the Quant-It kit (Life Technologies) and diluted to about 10,000 molecules per L to impose a complexity bottleneck. A complexity bottleneck sets the number of molecules that are amplified, matching the sequencing capacity to ensure that each molecule accumulates enough sequencing reads to assemble long synthetic reads. In this example, ten thousand molecules of adapter-ligated DNA were amplified by PCR using a PfuCx polymerase (Agilent Technologies™, Santa Clara, CA) or LongAmp Taq DNA polymerase (NEB) and a single primer (e.g., oligo 6 shown in Table 1 below) at 0.5 mM. The following thermocycling conditions were carried out: 92° C. for 2 minutes, followed by 40 cycles of 92° C. for 20 seconds, 55° C. for 20 seconds, and 68° C. for 3 minutes/kb, and followed by a final hold at 68° C. for 10 minutes.

The PCR products were purified with a Zymo column or a Qiagen Gel Extraction kit and eluted in 50 μL of buffer EB. Between 200 ng and one g of DNA was mixed with 1 unit of USER™ enzyme in a 45 μL reaction volume and incubated for 30 minutes at 37° C. Two L of 1:5 diluted dsDNA fragmentase (NEB™), 100 μg/mL bovine serum albumin, and 5 μL of dsDNA fragmentase buffer were added and the mixture incubated on ice for 5 minutes. 0.5-2 μL of dsDNA fragmentase (NEB) (volume adjusted based on amount and length of DNA to be fragmented) were then added and the mixture incubated at 37° C. for 15 minutes. The reaction was stopped by addition of 5 μL of 0.5 M EDTA and fragmentation was confirmed by the presence of a smear on an agarose gel. The DNA was purified with a Zymo column or 0.8 volumes of Ampure XP beads (Beckman Coulter™, Brea, CA), and eluted in 20 μL of buffer EB.

Two L of 10×NEBuffer 2 were added and fragmented DNA was incubated with 0.5 μL of “E. coli DNA ligase for fragmentase” (NEB) for 20 minutes at 20° C. Three units of T4 DNA polymerase (NEB), 5 units of Klenow fragment (NEB), and 50 μM of biotin-dCTP (Life Technologies) were added; and the reaction was incubated for 10 minutes at 20° C. Fifty M dGTP, dTTP, and dATP were added and the mixture was incubated for an additional 15 minutes, purified with a Zymo column or 1 volume of Ampure XP beads, eluted in 20 μL of elution buffer (buffer EB), and quantified by absorbance at 260 nm.

200-1000 ng of DNA at a final concentration of 1 ng/μL were mixed with 3000 units of T4 DNA ligase and T4 DNA ligase buffer to 1× and incubated at 16° C. for 16 hours. Linear DNA was digested by the addition of 10 units of T5 exonuclease and incubation at 37° C. for 60 minutes. Circularized DNA was purified with a Zymo column and eluted in 130 μL of buffer EB. The DNA was fragmented with an S2 disruptor (Covaris, Inc., Woburn, MA) to lengths of about 500 bp to about 800 bp.

Twenty μL of Dynabeads M-280 Streptavidin Magnetic Beads (Life Technologies) were washed twice with 200 μL of 2× B&W buffer (1× B&W buffer: 5 mM Tris-HCl (pH 7.5), 0.5 mM EDTA, 1 M NaCl) and resuspended in 100 μL of 2× B&W buffer. The DNA solution was mixed with this bead solution and incubated for 15 minutes at 20° C. The beads were washed three times with 200 μL of 1× B&W buffer, and twice in 200 μL of buffer EB. At this point, 15% (30 μL) of the beads were removed to a new tube for two-tube barcode pairing (see below). The remaining beads were resuspended in NEBNext™ End Repair Module solution (New England BioLabs Inc., Ipswich, MA) (42 μL water, 5 μL End Repair Buffer, and 2.5 μL End Repair Enzyme Mix), incubated at 20° C. for 30 minutes, washed three times with 200 μL of 1× B&W buffer, and then twice with 200 μL of buffer EB. The beads were resuspended in NEBNext A-tailing Module solution (NEB), incubated at 37° C. for 30 minutes, and washed three times with 200 μL of 1× B&W buffer, and then twice with 200 μL of buffer EB.

A 15 μM equimolar mixture of two oligonucleotides (e.g., oligos 8 and 9, as set out in Table 1 below) in 1×T4 DNA ligase buffer was incubated at 95° C. for 10 minutes and allowed to slowly cool to room temperature. The beads were resuspended in a solution comprising 5 μL of NEB Blunt/TA ligase master mix (NEB), 0.3 μL of 15 μM adapter oligo solution, and 4 μL of water. The mixture was incubated for 15 minutes at room temperature. The beads were washed three times with 200 μL of 1×B&W buffer, and twice with 200 μL of buffer EB. The beads were resuspended in a 50 μL PCR solution comprising 36 μL of water, 10 μL of 5×Phusion HF DNA polymerase buffer, 1.25 μL of each of 10 μM solutions of the standard Illumina Index and Universal primers (oligos 5 and 6 (set out below in Table 1), and 0.02 units/μL Phusion DNA polymerase (Thermo Fisher Scientific, Inc., Skokie, IL). The following thermocycling program was used: 98° C. for 30 seconds, followed by 18 cycles of 98° C. for 10 seconds, 60° C. for 30 seconds, and 72° C. for 30 seconds, and a final hold at 72° C. for 5 minutes. The supernatant was retained and the beads discarded.

The PCR product was purified with 0.7 volumes of Ampure XP beads and eluted in 10 μL buffer EB, or 500-900 bp fragments were size-selected on an agarose gel, gel-purified with the MinElute Gel Extraction kit, and eluted in 15 μL of buffer EB. The size distribution of the DNA was measured with an Agilent bioanalyzer and cluster-forming DNA was quantified by qPCR. The DNA fragments were sequenced on a MiSeq, NextSeq or HiSeq sequencer (Illumina™) with standard Illumina™ primers. Oligos 8 and 9, set out in Table 1 below, annealed to one another to form the asymmetric adapter. Oligos 10 and 11, set out in Table 1 below, were PCR primers that add the complete Illumina™ flowcell sequences. Sequences used in oligo 2, 10, and 11, as set out in Table 1 below, are from the Illumina™ Small RNA Kit. One oligo anneals to the asymmetric adapter, while the other oligo anneals to a region of the barcode adapter that is now on the interior of the fragment.

The Illumina™ sequences were taken from Illumina™ to ensure compatibility with the standard sequencing primer mix, but these sequences can be made longer or shorter or replaced entirely if corresponding custom sequencing primers are used. In this Example, 16-base random barcodes were used, but any length is adaptable for use. In the sequences used in this Example, there was a 2-base constant region outside the barcodes.

Moreover, two separate protocols were developed for barcode pairing, a two-tube protocol and a one-tube protocol. The one-tube protocol had the advantage of sample preparation occurring entirely in a single tube. A mixture of two or more barcode-containing adapters was ligated to the dT-tailed target fragments (e.g., a mixture of oligo 1 and oligo 2 as shown in Table 1). The adapters differed in their sequencing primer region. Sequences were derived from the Illumina™ Universal and Index primer sequences, respectively. As a result, approximately half of the target fragments had different sequencing regions in the adapters that ligate to the two ends. Following PCR, some fraction of the full-length copies avoided fragmentation, and circularization brought the two barcodes together. Downstream limited-cycle PCR (lcPCR) failed to amplify molecules that have the same adapter at each end because the identical sequencing regions outside the barcode regions will form a tight hairpin upon becoming single stranded. However, in molecules with different adapters at the ends, no hairpin formed, and addition of a primer complementary to the second sequencing region enabled amplification of the paired barcodes. In the computational pipeline, paired-barcode reads were identified, trimmed of adapter sequences, and parsed to extract the barcode pairs.

The two-tube protocol adds the complexity of splitting the library preparation into two tubes for the last third of the protocol, one tube to generate barcoded target reads and a second solely to generate paired barcode reads. One advantage is improved control of the fraction of the eventual short reads of each type. In this protocol, only one adapter sequence was used, so all target molecules ligated the same adapter at both ends. As a result, all molecules derived from circularized full-length amplicons formed a tight hairpin during lcPCR, and no paired-barcode reads were present in the main sequencing sample. Following attachment to streptavidin-coated beads and prior to ligation of asymmetric adapters, a fraction (˜15%) of the beads were moved to a second tube. SapI digestion cuts a site in the sequencing region (taken from the Illumina™ Multiplexing Sample Prep Oligo Only Kit), leaving sticky ends. Y-shaped adapters are ligated to the sticky ends to provide PCR annealing regions, and subsequent lcPCR adds the requisite sequencing adapter regions and a multiplexing index that allows barcode-pairing reads to be identified during analysis.

Two-tube barcode pairing: Bead-bound DNA was digested with 10 units of SapI in 1×CutSmart buffer in a 20 μL total volume for 1 h at 37° C. The beads were washed three times with 200 μL of 1×B&W buffer and twice with 200 μL of buffer EB. A 15 μM equimolar mixture of two oligonucleotides (oligos 12 and 13, as set out in Table 1 below) in 1×T4 DNA ligase buffer was incubated at 95° C. for 2 minutes and allowed to cool to room temperature over 30 minutes. The beads were resuspended in a solution comprising 5 μL of NEB Blunt/TA ligase master mix, 0.5 μL of 15 μM adapter oligo solution, and 4 μL of water. The mixture was incubated for 15 minutes at 4° C. and 15 minutes at 20° C. The beads were washed twice with 200 μL of 1×B&W buffer and twice with 200 μL of buffer EB. For amplification by limited-cycle PCR, the beads were resuspended in a 50 μL PCR solution comprising 36 μL of water, 10 μL of 5×Phusion HF DNA polymerase buffer, 1.25 μL of each of 10 μM solutions of two primers (oligos 11 and 14, as set out in Table 1 below, with oligo 14 (as shown in Table 1) selected to have a different multiplexing index than oligo 10 (as shown in Table 1) used above), and 0.02 units/L Phusion DNA polymerase (Thermo Fisher Scientific). The following thermocycling program was used: 98° C. for 30 seconds, followed by 18 cycles of 98° C. for 10 seconds, 60° C. for 30 seconds, and 72° C. for 30 seconds, and a final hold at 72° C. for 5 minutes. The supernatant was retained and the beads discarded. DNA was purified with 1.8 volumes of Ampure XP beads and eluted in 10 μL buffer EB. The expected product size of ˜170 bp was confirmed by agarose gel electrophoresis and Agilent bioanalyzer. Cluster-forming DNA was quantified by qPCR. The DNA fragments were mixed with the main library so as to comprise 1-5% of the total molecules, and sequenced on an Illumina MiSeq, NextSeq, or HiSeq with standard Illumina primer mixtures.

Single-tube barcode pairing: Oligos 1 and 2 (as shown in Table 1) were mixed, extended with oligo 6 (as shown in Table 1), and ligated to dT-tailed target fragments as above. The library preparation protocol was carried out as above, except that no extra barcode-pairing was completed. Limited-cycle PCR was performed with 1.25 μL of a 10 micromolar solution oligo 15, as set out in Table 1 below, in addition to oligos 10 and 11 as shown in Table 1.

### Complexity Determination:

The protocol includes quantification of doubly barcoded fragments prior to PCR. Doubly barcoded fragment concentration was estimated in three ways: quantitative PCR with a quenched fluorescent probe (oligo 19, as set out in Table 1 below), dilution series endpoint PCR, and quantification by next-generation sequencing. For the latter, barcoded molecules were purified and serially diluted. Four dilutions were amplified with oligo 6 and four versions of oligo 16, as set out in Table 1 below, containing different multiplexing index sequences. The resulting products were mixed and sequenced with 50-bp single-end reads on an Illumina™ MiSeq. Reads were demultiplexed and unique barcodes at each dilution were counted. When combined with the multiplexed library preparation strategy, which enables further demultiplexing on the basis of an index in the forward read, many samples can be quantified in a single MiSeq run.

TABLE 1
Oligonucleotide sequences

| OLIGO | | |
|---|---|---|
| NO. | Oligonucleotide Sequence | SEQ ID NO: |
| 1 | 5′-/5Phos/NNN GTTCAGAGTTCTACAGTCCGACGATC | SEQ ID NO: 1 |
| | NNNNNNNNNNNNNNNN CC | |
| | AGGAATAGTTATGTGCATTAATGAATGG CCGC-3′ | |
| 2 | 5′-/5Phos/NNN CCTACACGACGCTCTTCCGATCT | SEQ ID NO: 2 |
| | NNNNNNNNNNNNNNNN AC | |
| | AGGAATAGTTATGTGCATTAATGAATGG CCGC-3′ | |
| 3 | 5′-/5Phos/NNN CCTACACGACGCTCTTCCGATCT | SEQ ID NO: 3 |
| | NNNNNNNNNNNNNNNN AC | |
| | AATTCCTATCGTTCACGTCGTGT | |
| | CGCCATTTAGTGTCCAGTCTGA-3 | |
| 4 | 5′-/5Phos/NNN CCTACACGACGCTCTTCCGATCT | SEQ ID NO: 4 |
| | NNNNNNNNNNNNNNNN CC | |
| | AGGAATAGTTATGTGCATTAATGAATGG CGCC-3′ | |
| 5 | 5′-CCATTCAT/ideoxyU/AATGCACA/ideoxyU/ | SEQ ID NO: 5 |
| | AACTATTCC/3deoxyU/G*G-3′ | |
| 6 | 5′-CCATTCAT/ideoxyU/AATGCACA/ideoxyU/ | SEQ ID NO: 6 |
| | AACTATTCC/ideoxy U/G-3′ | |
| 7 | 5′-ACACGACG/ideoxyU/GAACGA/ | SEQ ID NO: 7 |
| | ideoxyU/AGGAAT/ideoxyU/G*T-3′ | |
| 8 | 5′-CCGAGAATTCCA*T-3′ | SEQ ID NO: 8 |
| 9 | 5′-/5Phos/TGGAATTCTCGG GTGCCAAGG-3′ | SEQ ID NO: 9 |
| 10 | 5′-CAAGCAGAAGACGGCATACGAGAT (Index) | SEQ ID NO: 10 |
| | GTGACTGGAGTT CCTTGGCACCCGAGAATTCCA-3′ | |
| 11 | 5′- | SEQ ID NO: 11 |
| | AATGATACGGCGACCACCGAGATCTACACTCTTTCCCT | |
| | ACACGACGCTCTTCCGATC*T-3′ | |
| 12 | 5′-ACACTCTTTCCCTACACGAC GCTCTTCC-3′ | SEQ ID NO: 12 |
| 13 | 5′-/5Phos/A*TC GGAAGAGC ACACGTCT | SEQ ID NO: 13 |
| 14 | 5′-CAAGCAGAAGACGGCATACGAGAT (Index) | SEQ ID NO: 14 |
| | GTGACTGGAGTTC AGACGTGTGCTCTTCCGATC*T-3′ | |
| 15 | 5′- | SEQ ID NO: 15 |
| | AATGATACGGCGACCACCGAGATCTACACGTTCAGAG | |
| | TTCTACAGTCCGA-3′ | |
| 16 | 5′-CAAGCAGAAGACGGCATACGAGAT (Index) | SEQ ID NO: 16 |
| | GTGACTGGAGTTC AGACGTGTGCTCTTCCGATC | |
| | CCATTCATTAATGCACATAACTATTCC-3′ | |
| 17 | 5′-CCATTCATTAATGCACATAACTATTCCT | SEQ ID NO: 17 |
| | GGNNNNNNNNNNNNNNNN | |
| | GATCGTCGGACTGTAGAACTCTGAAC T₃₀ VN-3′ | |
| 18 | 5′- GCGGCCATTCATTAATGCACATAACTATTCCT | SEQ ID NO: 18 |
| | GTNNNNNNNNNNNNNNNN | |
| | AGATCGGAAGAGCGTCGTGTAGG TrGrG+G-3′ | |
| 19 | 5′-/56-FAM/CCT ACA CGA /ZEN/CGC TCT TCC GAT | SEQ ID NO: 19 |
| | CT/3IABKFQ/-3′ | |
| 20 | 5′-NNN CCTACACGACGCTCTTCCGATCT | SEQ ID NO: 20 |
| | NNNNNNNNNNNNNNNN (Index) C | |
| | AGGAATAGTTATGTGCATTAATGAATGG CGCC-3′ | |

Key:
/5Phos/ = 5′ phosphate group
/ideoxyU/ = internal deoxyuracil base
/3deoxyU/ = 3′ deoxyuracil base
* = phosphorothioate linkage
rG = riboG
+G = locked nucleic acid G
N = mixture of A, T, G, and C
V = mixture of A, G, and C
T₃₀ = 30 consecutive Ts
lcPCR = limited-cycle PCR
Index = 6-base Illumina TruSeq Small RNA multiplexing index sequence
/56-FAM/ = probe fluorophore
/ZEN/ = probe quencher
/3IABKFQ/ = probe quencher

### Example 2—Testing Barcode Fidelity

Example 2 illustrates experiments carried out to test barcode fidelity. In general, a given barcode should be associated with a single target molecule, i.e., barcode fidelity. With barcode fidelity, every read tagged with that barcode should be derived from that single target molecule and should contain nucleotide sequence from that single target molecule alone.

Chimera formation during library preparation is problematic to barcode fidelity when sequencing a mixed population of target molecules. Once formed, chimeras are difficult to identify and filter out, and can confound assembly or lead to reconstruction of spurious sequences. Fortunately, the high coverage to which each target molecule is sequenced renders the method tolerant to a moderate level of chimera formation, in the same way that it ameliorates the effect of NGS error rates. Assuming 20-fold coverage at a chimera formation rate of 10%, half of the aligned calls at a given locus are erroneous only 0.005% of the time.

To test barcode fidelity of the method with homologous targets, a mixture of three linearized plasmids, each about 3 kb in length with homologous but distinct inserts, were sequenced. Each of the DNA plasmids, containing different mutants of the outer membrane protein A (OmpA) gene of E. coli, were purified from E. coli, linearized by restriction digestion, and mixed at known ratios. The resulting sample contained molecules of three known sequences, each at a different concentration. The target sequences were highly homologous and, thus, susceptible to recombination during PCR.

Following library preparation, sequencing, and barcode-mediated read sorting, the reads associated with each barcode were searched for short sequences unique to each target. The experiments showed that in the majority of cases, the contaminating reads were too few to confound analysis (see FIG. 2). About 80% of barcodes were confidently assigned to one target.

### Example 3—Sequencing Escherichia coli BL21

Genomic DNA was isolated from the model organism Escherichia coli BL21 using a MasterPure™ DNA Purification Kit (Epicentre™, Madison, WI) and sheared into fragments of an average length of about 3.5 kb using a HydroShear™ DNA Shearing System (Digilab™, Marlborough, MA). The fragment pool was converted to a sequencing-ready library following the protocol described herein and sequenced on a MiSeq sequencing instrument (Illumina™, Inc., San Diego, CA) with a 250 bp paired-end read reagent kit. De-multiplexed reads were processed using a custom computational pipeline, i.e., computer programs designed to process the sequencing data and assemble the synthetic long reads. Groups of reads sharing barcode sequences were assembled into long contiguous sequences or “contigs,” using the Velvet assembler, i.e., an algorithm package designed to assemble contigs from sequence information. See (http://www.ebi.ac.uk/-zerbino/velvet/velvet_poster.pdf).

743,538 paired-end read pairs were trimmed to remove barcodes, spurious sequences, adapter sequences, and regions of low quality. The read pairs were sorted into barcode-defined groups. Barcode-defined groups were assembled with Velvet into 644 contigs, wherein the contigs had lengths greater than 1,000 bp. The longest contig was 4,423 bp, and the end of the distribution is in concordance with the 3.5 kb average length of the sheared genomic fragments, indicating that complete target molecule sequences were reconstructed from some of the barcode groups using Velvet.

A histogram of barcode frequencies in the sequencing results revealed the expected bimodal distribution. There is a bimodal distribution because there are two types of barcodes: true barcodes (seen many times each) and false barcodes caused by sequencing errors (seen only a few times each). A peak at low numbers of times seen corresponds to spurious barcodes resulting from sequencing errors; these reads were discarded with no significant loss in efficiency. A second peak, centered near 500 times seen per barcode, corresponded to the true barcodes. This peak was much broader than the ideal peak that would result from random selection from an equal population of all barcodes, implying that PCR amplification is biased, over-amplifying some targets at the expense of others. This bias could be magnified by other parts of the protocol.

Bias, in some aspects, can be reduced by modifications to the protocol. For example, in some aspects, bias is reduced by adding a linear amplification phase prior to exponential PCR, or by optimizing PCR conditions (e.g., primer sequences, extension times, annealing temperatures, etc.). Still, given the low and rapidly declining cost of sequencing, the current levels of bias do not result in prohibitive inefficiency.

The relationship between the number of reads associated with a barcode and the longest contig assembled from those reads indicated that additional reads aid assembly (as expected) up to about 1000 reads. However, not only do barcodes that are seen more than 1000 times gain no extra advantage, the length of their longest contigs drops off. In some aspects, this may be a result of extra sequencing errors that confound assembly accumulating in excess reads, or indicate that the most frequently seen barcodes derive from spurious sequences.

The complexity bottleneck (a restriction on the number of barcoded molecules) imposed upon the mixed DNA population by dilution prior to PCR can be chosen for each experiment as a function of the length of the target molecules and the number of sequencing reads available. For example, in this experiment, the true complexity bottleneck was estimated to have been on the order of 1000 (about 700,000 reads divided by ˜500 reads per barcode). Thus, the complexity (number of barcoded molecules) is bottlenecked (restricted) prior to PCR to optimize sequence assembly. If too many molecules are amplified in PCR, the sequencing reads are spread out among them to the point that full-length sequences cannot be assembled. If too few, then fewer than an optimal number of sequences are assembled. The choice of complexity depends on the number and length of reads to be generated, the length of the target molecules, and whether barcode pairing is used. In various aspects, determining the number of barcoded molecules in a sample is done by qPCR, dilution-series PCR, digital PCR, specific degradation of molecules lacking two adapters followed by quantification, or sequencing.

A BLAST search of the assembled contigs against known genomes confirmed that the majority of the contigs aligned to the E. coli genome with high accuracy. Contigs of length greater than 250 bp were submitted to the query. The contigs that aligned with the reference genome matched with 99.95% agreement, for an error rate of 0.05%. It is notable that this 0.05% error rate represents a ceiling on the error rate of the method, because the sequenced strain may have accumulated mutations that differentiate it from the reference, and because there is potential to optimize the assembly algorithm for greater accuracy.

In every barcode pool alignment that was examined, about 80% of the reads aligned within the same 3-4 kb region. The other 20% aligned to other areas of the genome in a seemingly random manner, likely as a result of intermolecular circularization during library preparation. This fraction is reducible through optimization of the circularization conditions, but this randomly scattered minority of fragments does not typically confound assembly or other applications of the method.

### Example 4—Sequencing Geoglobus ahangari

Genomic DNA was isolated from the archaea Geoglobus ahangari using the Masterpure™ DNA Purification Kit (Epicentre™) and sheared into fragments of an average length of 3.5 kb using a HydroShear DNA Shearing System (Digilab™). The fragment pool was converted to a sequencing-ready library according to the protocol provided above and sequenced on a MiSeq instrument (Illumina™) with a 250 bp paired-end read reagent kit. De-multiplexed reads were processed using a custom computational pipeline, as described herein. Groups of reads sharing barcode sequences were assembled into contigs using the Velvet assembler. 2.3 million paired-end read pairs were trimmed to remove barcodes, spurious sequences, adapter sequences, and regions of low quality, and sorted into barcode-defined groups. Using the Velvet assembler, the resultant barcode groups were assembled into 1497 contigs of lengths greater than 1,000 bp. The longest contig was 4,507 bp, and the end of the distribution is in concordance with the 3.5 kb average length of the sheared genomic fragments, indicating that Velvet was able to reconstruct complete target molecule sequences from some of the barcode groups.

Geoglobus ahangari contigs were used to improve an existing, incomplete draft genome for this organism. The draft genome contained 50 disconnected contigs. Long reads from the method disclosed herein allowed the 50 disconnected contigs to be collapsed into 30 contigs, containing no unresolved (“N”) bases. This experiment demonstrated that the long contigs derived from methods of the disclosure dramatically improved the draft genome of Geoglobus ahangari by resolving ambiguities in short-read assemblies.

The bimodal distribution of barcode frequencies was less pronounced in the Geoglobus data, indicating potentially more severe PCR bias compared to the E. coli data. The true complexity bottleneck is estimated to have been on the order of about 4000 (about 2.3 million reads divided by ˜500 reads per barcode).

### Example 5—Sequencing Tuberosum solanum

Genomic DNA was isolated from a doubled monoploid variety of an important food crop, i.e., Tuberosum solanum (the potato), and sheared into fragments of an average length of 3.5 kb using a HydroShear DNA Shearing System (Digilab™). The fragment pool was converted to a sequencing-ready library according to the protocol set out above and sequenced on a MiSeq™ instrument (Illumina™) with a 250 bp paired-end read reagent kit. De-multiplexed reads were processed using a custom computational pipeline, as described herein. Groups of reads sharing barcode sequences were assembled using the Velvet assembler.

10.2 million paired-end read pairs were trimmed to remove barcodes, spurious sequences, adapter sequences, and regions of low quality, and sorted into barcode-defined groups. Using the Velvet assembler, the resultant barcode groups were assembled into 1,508 contigs of length greater than 1,000 bp. The longest contig was 5,249 bp, and the end of the distribution was in concordance with the 3.5 kb average length of the sheared genomic fragments, indicating that Velvet was able to reconstruct complete target molecule sequences from some of the barcode groups.

The sequencing results revealed the expected bimodal distribution. The true complexity bottleneck was estimated to have been on the order of about 4000 (about 10.2 million reads divided by ˜3000 reads per barcode).

Assembled reads were analyzed further using bioinformatics. A blind test was carried out because the experimenters did not have access to the potato reference genome during contig assembly. The potato contigs were aligned to an existing draft genome maintained by the Potato Genome Consortium. Approximately 70-90% of the contigs aligned to the reference genome, depending on the stringency of the alignment parameters (minimum 98% agreement). The high sequence agreement between the long contigs and the draft genome highlighted the accuracy of contigs generated by methods of the disclosure, in contrast to previously known long-read technology. A Basic Local Alignment Search Tool (BLAST, NIH) search returned hits to potato, as well as related organisms, including tomato and nightshade. Potato is a tetraploid organism. Long reads, such as those obtained by methods of the disclosure, are instrumental to resolving the haplotype of each chromosome.

### Example 6—Sequencing Escherichia coli Strain MG1655

Sequencing libraries were prepared from genomic DNA isolated from E. coli strain MG1655. Genomic DNA was sheared and size-selected to a range of about 5-10 kb. About 8 million 150 bp paired-end read pairs were filtered and trimmed to remove barcodes, adapter sequences, and regions of low quality and then sorted into barcode-delineated groups, as described herein. Barcode pairing resolved 1,186 distinct barcode pairs, whose read groups were merged prior to assembly. Independent assembly of each group with the SPAdes assembler (Bankevich et al., J. Computational Biology 19(5): 455-77, 2012) yielded 2,826 contigs of length greater than 1,000 bp.

To determine the fidelity of assembly, the largest contig assembled from each barcode-defined group was aligned to the MG1655 reference genome (Hayashi et al., Mol. Syst. Biol. 2:0007, 2006). Alignment of grouped reads to the reference genome showed a non-uniform distribution of coverage across the fragment length, with coverage dropping off along the length of the target sequence. Barcode pairing reduced the impact of the coverage drop because coverage from one barcode is high in the region where coverage from its pair is low. Coverage is the number of short reads that align to a given location on the long target sequence. Coverage drops from one end of the target to the other, presumably because circularization is less efficient for longer molecules. Coverage from reads with the partner barcode is a mirror image: high on the other end, and dropping toward the first end. The sum of the two profiles is therefore relatively smoothed. This experiment showed that assembly of longer molecules requires high average read depths. Merging the paired read groups resulted in a smoother distribution of coverage (see FIG. 1B.)

The length distribution of the assembled contigs had an N50 (half of the total assembled bases are in contigs greater than the N50) of 6 kb and a maximum assembly length of 11.6 kb (see FIG. 1C). The error rate when contigs were aligned back to the reference MG1655 genome was only about 0.1%. Thus, the experiment showed that the method described herein was used to assemble contigs with an N50 of 6 kb with about 99.9% accuracy.

### Example 7—Sequencing Gelsemium sempervirens

Sequencing libraries were prepared from genomic DNA isolated from Carolina jasmine (Gelsemium sempervirens), a plant with a complex and previously unsequenced genome. 149,447 contigs longer than 1 kb, with an N50 of 4 kb, were assembled. The assembled long reads aligned with high stringency to a draft assembly of the Gelsemium sempervirens genome, and increased the maximum scaffold length from about 197,779 bp to about 365,589 bp. Thus, the experiment showed that the method described herein was used to assemble contigs with an N50 of 4 kb (see FIG. 1C), and was useful in assembling a large portion of a previously unsequenced genome.

### Example 8—Library Preparation for Synthetic Long Read Assembly from mRNA Samples

Full-length reverse transcripts were prepared with primers, where the primers included oligo 17 and oligo 18, as set out in Table 1 above, respectively. Barcoded full-length reverse transcripts were then processed and sequenced, starting from library quantification. The barcoded cDNA product was amplified, broken, circularized, and prepared for sequencing. From mRNA isolated from HCT116 and HepG2 cells, 28,689 and 16,929 synthetic reads were assembled, respectively, of lengths between 0.5 and 4.6 kb. Synthetic reads spanned multiple splice junctions, with a median of 2.0 spanned junctions per synthetic read for both samples and a maximum of 35 spanned junctions. Examination of the synthetic reads revealed examples of differential splicing between the HCT116 and HepG2 cell lines, as well as a novel transcript in the HCT 116 cell line.

### Example 9—Multiplexed Sample Preparation

Two E. coli strains were isolated from each of twelve recombination treatment populations (See e.g., Souza et al. Journal of Evolutionary Biology 10:743-769, 1997). Genomic DNA was isolated from each of the twenty-four strains, sheared, end-repaired, and dT-tailed as described above in separate tubes. Twenty-four barcode adapters (oligo 20, as set out in Table 1 above), identical except for distinct 6-bp multiplexing index regions adjacent to the barcode sequence, were prepared and ligated to the genomic fragments as described above. Adapter-ligated DNA was PCR amplified as above. Purified PCR products were quantified, and equal amounts were combined into a single mixture. This mixture was prepared for sequencing following the other parts of the above protocol. Sequencing reads were demultiplexed by project according to standard 6-bp index read, then further demultiplexed by strain according to the barcode-adjacent multiplexing index identified in the forward read, sorted by barcode, and assembled in parallel. The summed lengths of the synthetic reads longer than 1 kb exceeded twofold genome coverage for sixteen out of the twenty-four strains, with a median genome coverage of 2.3-fold and median N50 of 4.1 kb.

### Example 10—Fragment Generation Based on Extension of Random Primers

Fragments with randomly determined ends are created by annealing primers of random or partially random sequences. Each such primer anneals to a complimentary region of the target molecule and is extended by a polymerase. The polymerase is capable of strand displacement. The targets are or are not amplified beforehand. A mixture including template molecules and random primers is melted at 95° C. and quenched to 0° C. to allow primer annealing. Primers complementary to the adapter ends of the target are present or are added, and prime the single-stranded DNA synthesized following random priming at its 3′ end. Extension by a DNA polymerase generates double-stranded DNA fragments with the known adapter end sequence at one end and a random sequence from the interior of the target molecule at the other end. Multiple rounds of this linear amplification and fragment generation are performed. These additional rounds are performed by heating the mixture to e.g., 95° C. to melt the double-stranded DNA duplexes, cooling to promote random primer annealing, and if necessary, adding additional DNA polymerase. The target molecule adapters contain one or more biotinylated nucleotides that allow them to specifically bind to streptavidin-coated beads, so that the newly generated fragments can be easily separated from the original targets between rounds of amplification. The random primers contain defined sequences at their 5′ end and random sequences at their 3′ end, so that the resulting ssDNA or dsDNA contains known sequences at both ends. Fragments are subsequently amplified by PCR using one or more primers complementary to the known end sequences. DNA fragments created by linear or exponential amplification contain known end sequences that are reverse complements of each other and contain one or more deoxyuracil bases in the 5′ ends. A combination of uracil-DNA glycosylase (UDG) and exonuclease VIII can then be used to remove the 5′ ends, leaving long single-stranded complimentary sequences that can anneal to increase the efficiency of intramolecular circularization. Treatment with UDG and exonuclease VIII is preceded by treatment with Klenow fragment or a similar enzyme to remove nontemplated deoxyadenosine bases added to the 3′ ends during extension. The known end sequences contain sequences that can be recognized by recombinase enzymes that circularize the fragment by recombination. Circularization is by blunt-end ligation.

Circularized fragments are fragmented by mechanical methods and prepared for sequencing by ligating adapters and performing lcPCR as described herein.

Circularized fragments are amplified by rolling-circle amplification (RCA) or hyperbranching rolling-circle amplification (HRCA). RCA or HRCA is primed with random primers or partially random primers. Amplification is performed in the presence of up to 100% dUTP in place of dTTP, to allow the product to be specifically degraded later. RCA or HCRA is followed by mechanical fragmentation, adapter ligation, and PCR as described herein.

PCR is primed with one primer complementary to the defined sequence at the 5′ end of the partially random primer used for RCA or HRCA, and a second primer complementary to a sequence in the barcode adapter proximal to the barcode sequence RCA or HCRA products containing deoxyuracil are subsequently degraded to enrich for PCR products.

With reference to FIG. 8A, a mixture of target DNA molecules, with barcode adapters attached to the ends according to methods described herein, is prepared with the desired complexity (number of distinct molecules). The barcode adapters contain an end region of defined sequence (X), a degenerate barcode region (B) that is different for every target molecule but defined for a given individual molecule, and a defined region (I₁) complementary to some or all of one of the two eventual sequencing primers, such as a standard sequencing primer (e.g., Illumina™) or a custom primer. Molecules are amplified by linear or exponential methods to create 10¹-10⁵ copies of each uniquely barcoded molecule. The target molecules are then melted into single-stranded DNA by heating or exposure to alkaline or other denaturing conditions. One or more random or partially random primers are then annealed along the length the target molecules by rapid quenching to 0-4° C. The primers depicted here are partially random, with a random 3′ region and a defined 5′ region (e.g., sequence Y).

Continuing with FIG. 8A and FIG. 8B, a strand-displacing DNA polymerase, such as Bst DNA polymerase, is added to the primer-annealed target DNA mixture. The temperature is ramped or stepped up to 65° C., and the polymerase extends each of the random 3′ primer ends annealed along the length of the target molecule, displacing extended molecules in front of it as it goes and releasing them into solution. One end of the newly synthesized single-stranded DNA molecules is defined by the partially random primer and contains the Y sequence followed by a sequence complementary to the region of the target molecule to which a specific primer from the degenerate mixture annealed. The other end is defined by a sequence complementary to the end sequence of the target molecule, which comprises I₁-B-X. A primer with a sequence complementary to X is present in the mixture, and is designed with an annealing temperature greater than 65° C., allowing it to anneal to the ends of the newly synthesized displaced molecules and prime synthesis of the second strand, creating double-stranded DNA. The result is a collection of target fragments, with no mechanical or enzymatic shearing needed. If desired, multiple cycles of melting, annealing, and strand-displacement amplification can be performed to increase the yield of DNA. If desired, deoxyadenosine overhangs are then added by the Bst polymerase in a template-independent fashion and can later be removed by incubation with. Klenow DNA polymerase to create blunt-ended dsDNA.

Continuing with FIG. 8A and FIG. 8B, fragments synthesized can be circularized by blunt-end ligation. Alternatively, to improve circularization efficiency of long fragments, sticky-end ligation can be performed, as shown here. If sequences X and Y in the partially random primers and the second-strand primers are synthesized so that they contain deoxyuracil bases, the USER™ enzyme mix (UDG and endonuclease VIII) can excise the 5′ ends of each strand of the dsDNA to leave sticky ends of programmable length. If X and Y are reverse complements, the sticky ends will be complementary, and will anneal to one another to promote ligation.

### Example 11—Preparing Library Molecules Compatible with an Element Biosciences Flowcell

A large number of short reads were generated which were then assembled into longer length sequencing reads (e.g., the so called synthetic long reads). A synthetic long read workflow was performed to analyze 16S rRNA from bacterial or environmental samples. The analysis was conducted by fragmenting DNA from various high-complexity sources including Rhodobacter sphaeroides (ATCC strain) and environmental gDNA. In another study, DNA encoding antibody chains (i.e., lower complexity) were analyzed.

Two different types of libraries were prepared. One type was compatible for sequencing on an Illumina™ NextSeq 550, and the other type was compatible for sequencing on an AVITI™ sequencing apparatus from Element Biosciences. When the Element Biosciences library was prepared, the Illumina™ universal adaptor sequences were substituted for corresponding universal adaptor sequences that are compatible with sequencing on an Element Biosciences massively parallel sequencing platform, including for example Element Biosciences] universal surface capture primer, universal surface pinning primer, universal forward sequencing primer binding site, and/or universal reverse sequencing primer binding site. The tripartite adaptor included an outer PCR primer region, an inner sequencing primer binding site for an Element Bioscience sequencing workflow, and a central UMI/barcode region. The sequencing primer binding site included a portion of the sequence 5′-CGTGCTGGATTGGCTCACCAGACACCTTCCGACAT-3′ (SEQ ID NO:22) which comprises a forward sequencing primer binding site for an Element Biosciences sequencing workflow. In some embodiments, the sequencing primer binding site can include the full-length sequence 5′-CGTGCTGGATTGGCTCACCAGACACCTTCCGACAT-3′ (SEQ ID NO:22) which comprises a forward sequencing primer binding site for an Element Biosciences sequencing workflow. The tripartite adaptor was appended to one end of nucleic acid fragments of interest to generate adaptor-fragment molecules. The adaptor-fragment molecules were amplified. The amplified adaptor-fragment molecules were fragmented (e.g., randomly fragmented) to generate molecules having unknown end sequences. The randomly fragmented molecules were circularized to generate circular molecules having the UMI/barcode in proximity to the unknown end sequences. The circularized molecules were randomly fragmented to generate linear molecules some of which carry at least a portion of the tripartite adaptor, where some of the randomly fragmented molecules also carry an unknown end sequence in proximity to a UMI/barcode. Thus, a given UMI/barcode sequence was distributed to random positions within each parent library molecule. The linear molecules were appended with universal adaptors carrying a reverse sequencing primer binding site (150) with the sequence 5′-ATGTCGGAAGGTGTGCAGGCTACCGCTTGTCAACT-3′ (SEQ ID NO:23). The linear molecules, now carry a forward sequencing primer binding site (140), an insert region (110), and a reverse sequencing primer binding site (150). The linear molecules were appended with the surface pinning primer binding site (120) and a left sample index sequence (160), and the surface capture primer binding site (130) and right sample index sequence (170), using tailed PCR primers. The sequence of the surface pinning primer binding site (120) was 5′-CATGTAATGCACGTACTTTCAGGGT-3′ (SEQ ID NO:21). The sequence of the surface capture primer binding site (130) was 5′-AGTCGTCGCAGCCTCACCTGATC-3′ (SEQ ID NO:24).

The final linear molecules were Element-compatible library molecules which comprise (i) a surface pinning primer binding site (120), (ii) a left sample index sequence (160), (iii) a forward sequencing primer binding site (140), (iv) a UMI sequence (180), (v) an insert sequence (e.g., sequence of interest) (110), (vi) a reverse sequencing primer binding site (150), (vii) a right sample index sequence (170) which optionally includes a 3-mer random sequence, and (viii) a surface capture primer binding site (130) (e.g., see FIG. 10).

The Element-compatible library molecules were circularized by hybridizing to single-stranded splint strands (200) to generate covalently closed circular molecules each having a nick (e.g., see FIG. 10). The single-stranded splint strands (200) comprise the sequence

5′-ACCCTGAAAGTACGTGCATTACATGGATCAGGTGAGGCTGCGACGACT-3′(SEQ ID NO:27). The nick was enzymatically closed to generate covalently closed circular molecules (e.g., see FIGS. 10 and 12). The covalently closed circular molecules were distributed by flowing onto a flowcell coated with a hydrophilic polymer coating having a plurality of surface capture primers and surface pinning primers immobilized thereon. The covalently closed circular molecules which were distributed on the coated flowcell were subjected to a rolling circle amplification reaction to generate a plurality of concatemer molecules that were immobilized to the surface capture primers tethered to a hydrophilic coating (e.g., see FIG. 28). The immobilized concatemer molecules were subjected to multiple cycles of a two-stage sequencing reaction that employs detectably labeled multivalent molecules and non-labeled nucleotide analogs.

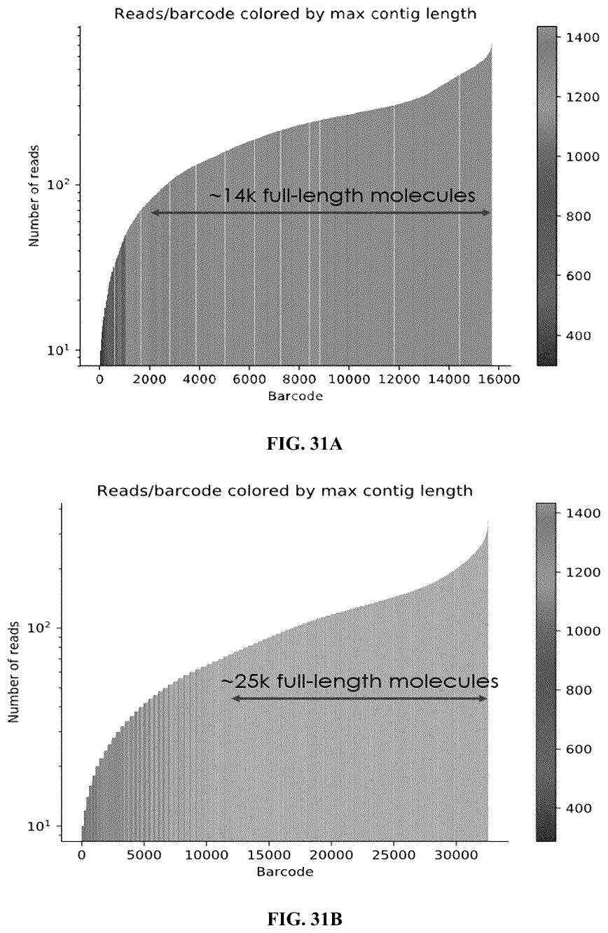

The short sequencing reads that carried the same UMI/barcode sequence were binned together and informatically reassembled back into full length sequences (contigs) of the original parent molecule (e.g., appended with a UMI/barcode), thereby generating synthetic long reads. Each contig was assembled from a collection of short reads having the same UMI/barcode sequence, indicating a shared origin from an original parent molecule. A sufficient number of short read coverage across a full-length gene (e.g., 16S rRNA or antibody chain) makes it possible to reassemble the entire sequence of the gene by assembly of short reads with the same UMI/barcode. FIGS. 31-36 show bar graphs in which the x-axis indicates individual UMIs that represent original molecules arranged from shortest to longest. The y-axis indicates the number of reads that shared the same UMI (e.g., binned by UMI) that were used to assemble that contig. The shading of the bar (ranging from light to dark) indicates contig length. Full length contigs are displayed in light shading while non-full length contigs are displayed darker. The transition point, from dark to light, and indicated by the left side of the double-arrow, indicates the point where the synthetic assembly achieved complete reconstruction of the nucleic acid sequence of interest. The bar graphs show the relationship between the number of reads needed to generate a contig (e.g., of any length) and the fraction of total contigs (e.g., based on UMIs). The bar graphs in FIG. 31-36 are contig length histograms showing all of the UMI-tagged contigs as a function of the number of short reads required to assemble full length contigs. The target complexity was about 20,000 UMI-tagged molecules. The bar graphs in FIG. 31-36 compared contig length resulting from two different sequencing reactions, including a fluorescently-labeled chain terminator nucleotide sequencing method (e.g., Illumina™ NextSeq 550™), and two-stage sequencing method (e.g., AVITI™ sequencing from Element Biosciences). The AVITI™ sequencing runs were down-sampled to permit a comparison with the shallower sequencing depth of the NextSeq 550™ sequencing runs.

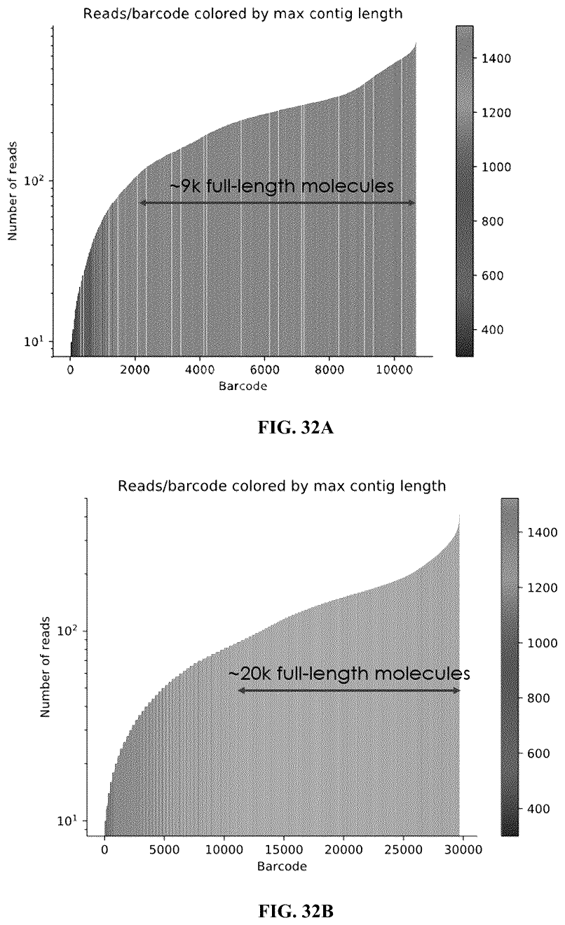

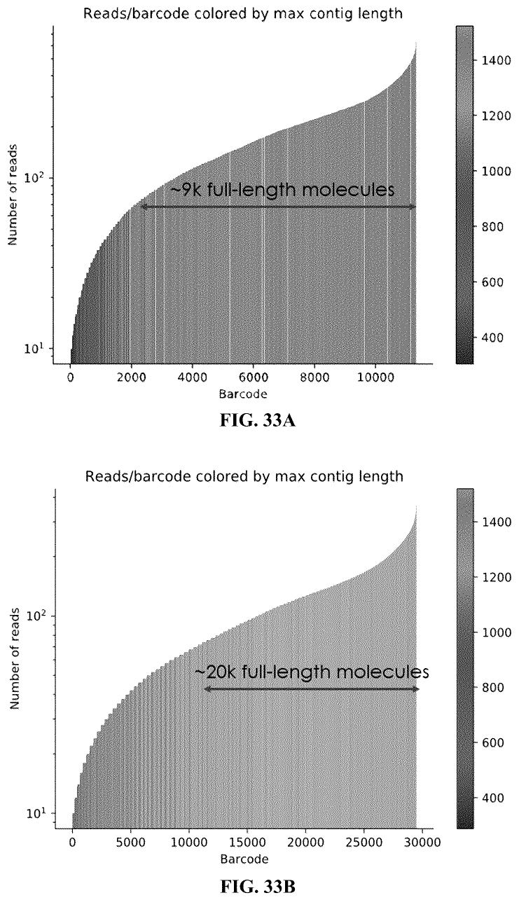

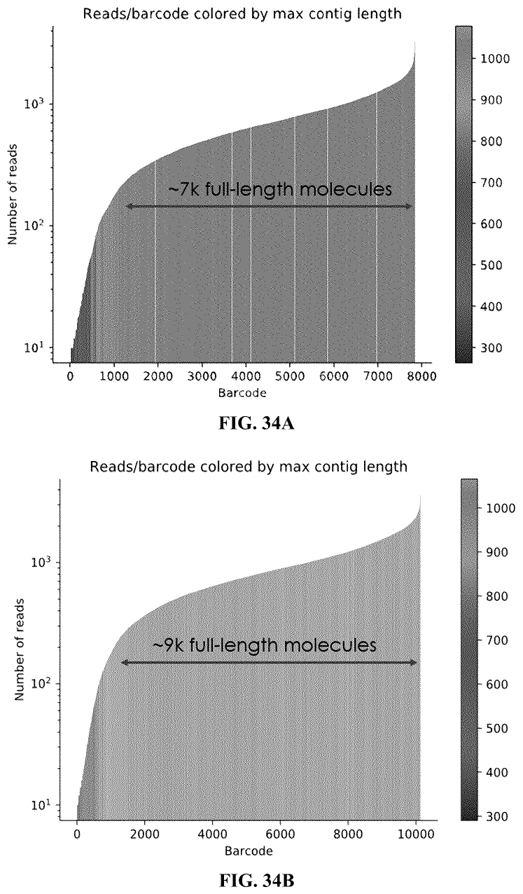

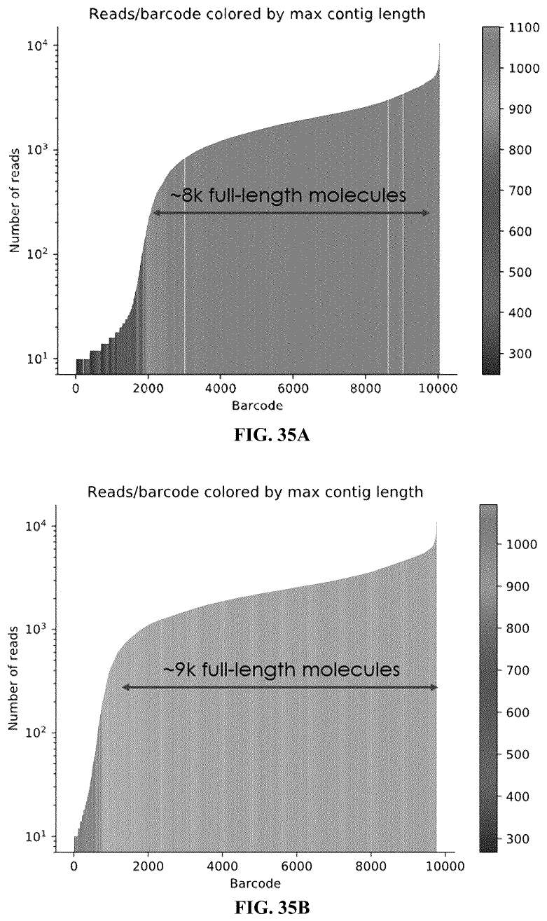

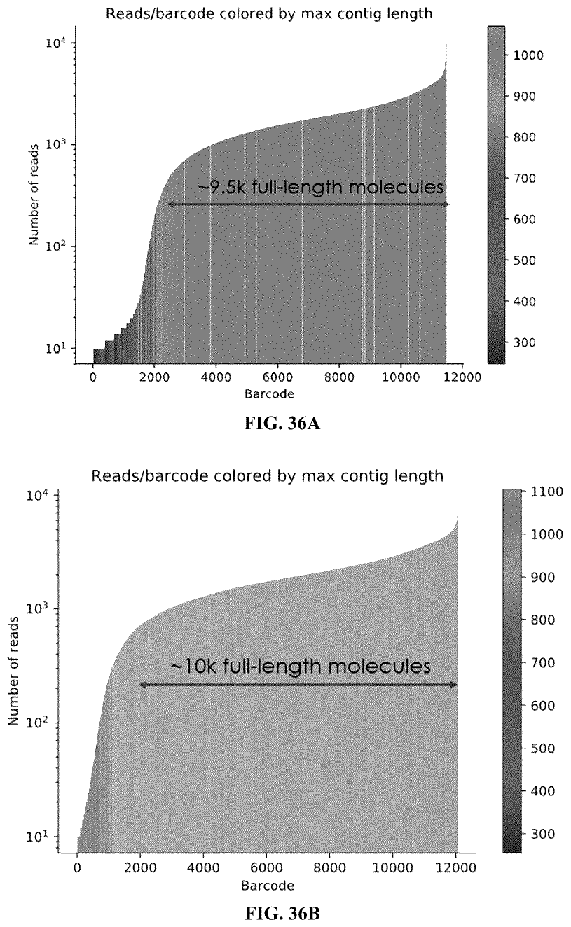

As shown in FIG. 31A and FIG. 31B, sequencing of a Rhodobacter sphaeroides sample on AVITI™ (FIG. 31B) resulted in about twice as many reads as sequencing on NextSeq 550™ (FIG. 31A). Similarly, sequencing of a heterogenous environmental gDNA sample on AVITI™ (FIG. 32B, FIG. 33B) resulted in about twice as many reads as sequencing on NextSeq 550™ (FIG. 32A, FIG. 33A). Contig lengths were about twice as long for those on AVITI™ as compared to NextSeq 550™. As shown in FIG. 34A and FIG. 34B, FIG. 35A and FIG. 35B, and FIG. 36A and FIG. 36B, AVITI™ and NextSeq 550™ performed comparably when sequencing an antibody.

### INCORPORATION BY REFERENCE

All publications and patent applications mentioned in this specification are herein incorporated by reference to the same extent as if each individual publication or patent application was specifically and individually indicated to be incorporated by reference.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

What is claimed is:

1. A method for obtaining nucleic acid sequence information from a nucleic acid molecule comprising a target nucleotide sequence by assembling a series of nucleic acid sequences into a longer nucleic acid sequence, said method comprising:
(a) attaching a first adapter at the 5′ end and/or the 3′ end of a linear nucleic acid molecule, said first adapter comprising an outer polymerase chain reaction (PCR) primer region or nucleic acid amplification region, an inner sequencing primer region, and a central barcode region to each end of a plurality of linear nucleic acid molecules to form barcode-tagged molecules;
(b) replicating the barcode-tagged molecules to obtain a library of barcode-tagged molecules;
(c) breaking the library of barcode-tagged molecules, thereby generating a first set of linear, barcode-tagged fragments, each comprising the barcode region at one end and a region of unknown sequence at the other end;
(d) circularizing the first set of linear, barcode-tagged fragments comprising the barcode region at one end and a region of unknown sequence from an interior portion of the target nucleotide sequence at the other end, thereby bringing the barcode region into proximity with the region of unknown sequence and generating circularized, barcode-tagged fragments;
(e) fragmenting the circularized, barcode-tagged fragments into a second set of linear, barcode-tagged fragments;
(f) attaching a second adapter to each end of each of the second set of linear, barcode-tagged fragments to form double adapter-ligated barcode-tagged nucleic acid fragments, each double adaptor-ligated barcode-tagged nucleic acid fragment comprising a plurality of library molecules (100) comprising: (i) a surface pinning primer binding site (120), (ii) a left sample index sequence (160), (iii) a forward sequencing primer binding site (140), (iv) a left unique molecular index (UMI) sequence (180), (v) an insert sequence (110), (vi) a reverse sequencing primer binding site (150), (vii) a right sample index sequence (170), and (viii) a surface capture primer binding site (130);
(g) replicating the double adapter-ligated barcode-tagged nucleic acid fragments;
(h) sequencing the double adapter-ligated barcode-tagged nucleic acid fragments;
(i) sorting a series of sequenced nucleic acid fragments into independent groups of reads; and
(j) assembling each independent group of reads into the longer nucleic acid sequence,
thereby obtaining the nucleic acid sequence information.

2. The method of claim 1, further comprising: generating single stranded library molecules from the plurality of library molecules (100).

3. The method of claim 1, wherein the right sample index sequence (170) includes a 3-mer random sequence.

4. The method of claim 1, wherein step (g) comprises replicating all of the double adapter-ligated barcode-tagged nucleic acid fragments.

5. The method of claim 1, further comprising: forming a plurality of library-splint complexes (300) comprising:
i) providing a plurality of single-stranded splint strands (200) wherein individual single-stranded splint strands (200) in the plurality comprise a first region (210) that is capable of hybridizing with the at least a first left universal adaptor sequence (120) of an individual library molecule, and a second region (220) that is capable of hybridizing with the at least a first right universal adaptor sequence (130) of the individual library molecule;
ii) hybridizing the plurality of single-stranded splint strands (200) with plurality of single-stranded nucleic acid library molecules (100) such that the first region of one of the single-stranded splint strands (210) anneals to the at least first left universal adaptor sequence (120) of the library molecule, and such that the second region of the single-stranded splint strand (220) anneals to the at least first right universal sequence (130) of the library molecule, thereby circularizing individual library molecules to form a plurality of library-splint complexes (300) having a nick between the terminal 5′ and 3′ ends of the library molecule, wherein the nick is enzymatically ligatable; and
iii) ligating the nick in the plurality of library-splint complexes (300) thereby generating a plurality of covalently closed circular library molecules (400).

6. The method of claim 5, further comprising: (iv) distributing the plurality of covalently closed circular library molecules (400) onto a support having a plurality of surface primers immobilized on the support, under a condition suitable for hybridizing individual covalently closed circular library molecules (400) to individual immobilized surface primers thereby immobilizing the plurality of covalently closed circular library molecules (400).

7. The method of claim 6, further comprising:
(v) contacting the plurality of immobilized covalently closed circular library molecules (400) with a plurality of strand-displacing polymerases and a plurality of nucleotides, under a condition suitable to conduct a rolling circle amplification reaction on the support using the plurality of surface primers as immobilized amplification primers and the plurality of covalently closed circular library molecules (400) as template molecules, thereby generating a plurality of immobilized nucleic acid concatemer molecules.

8. The method of claim 7, wherein step (h) comprises sequencing the plurality of immobilized nucleic acid concatemer molecules.

9. The method of claim 8, wherein the sequencing the plurality of immobilized nucleic acid concatemer molecules further comprises:
a) contacting the plurality of immobilized concatemer molecules with (i) a plurality of sequencing polymerases and (ii) a plurality of the soluble sequencing primers, wherein the contacting is conducted under a condition suitable to form a plurality of complexed polymerases each comprising a sequencing polymerase bound to a nucleic acid duplex wherein the nucleic acid duplex comprises a concatemer molecule hybridized to a soluble sequencing primer;
b) contacting the plurality of complexed sequencing polymerases with a plurality of nucleotides under a condition suitable for binding at least one nucleotide to a complexed sequencing polymerase, wherein the plurality of nucleotides comprises at least one nucleotide analog labeled with a fluorophore and having a removable chain terminating moiety at the sugar 3′ position;
c) incorporating at least one nucleotide into the 3′ end of the hybridized sequencing primers thereby generating a plurality of nascent extended sequencing primers; and
d) detecting the incorporated nucleotide and identifying the nucleo-base of the incorporated nucleotide.

10. The method of claim 9, wherein the sequencing the plurality of immobilized nucleic acid concatemer molecules further comprises:
a) contacting the plurality of immobilized concatemer molecules with (i) a plurality of sequencing polymerases and (ii) a plurality of the soluble sequencing primers, wherein the contacting is conducted under a condition suitable to form a plurality of first complexed polymerases each comprising a sequencing polymerase bound to a nucleic acid duplex, wherein the nucleic acid duplex comprises a concatemer molecule hybridized to a soluble sequencing primer;
b) contacting the plurality of complexed sequencing polymerases with a plurality of detectably labeled multivalent molecules to form a plurality of multivalent-complexed polymerases, under a condition suitable for binding complementary nucleotide units of the multivalent molecules to at least two of the plurality of first complexed polymerases thereby forming a plurality of multivalent-complexed polymerases, and the condition inhibits incorporation of the complementary nucleotide units into the sequencing primers of the plurality of multivalent-complexed polymerases, wherein individual multivalent molecules in the plurality of multivalent molecules comprise a core attached to multiple nucleotide arms and each nucleotide arm is attached to a nucleotide unit;
c) detecting the plurality of multivalent-complexed polymerases; and
d) identifying the nucleo-base of the complementary nucleotide units that are bound to the plurality of first complexed polymerases in the plurality of multivalent-complexed polymerases, thereby determining the sequence of the nucleic acid template.

11. The method of claim 10, further comprising:
e) dissociating the plurality of multivalent-complexed polymerases and removing the plurality of first sequencing polymerases and their bound multivalent molecules, and retaining the plurality of nucleic acid duplexes;
f) contacting the plurality of the retained nucleic acid duplexes of step (e) with a plurality of second sequencing polymerases, wherein the contacting is conducted under a condition suitable for binding the plurality of second sequencing polymerases to the plurality of the retained nucleic acid duplexes, thereby forming a plurality of second complexed polymerases each comprising a second sequencing polymerase bound to a retained nucleic acid duplex;
g) contacting the plurality of second complexed polymerases with a plurality of non-labeled nucleotides, wherein the contacting is conducted under a condition suitable for binding complementary nucleotides from the plurality of nucleotides to at least two of the second complexed polymerases of step (f) thereby forming a plurality of nucleotide-complexed polymerases and the condition is suitable for promoting incorporation of the bound complementary nucleotides into the sequencing primers of the nucleotide-complexed polymerases.

12. The method of claim 10, wherein the method comprises:
a) binding a first universal nucleic acid primer, a first DNA polymerase, and a first multivalent molecule to a first portion of the concatemer molecules, thereby forming a first binding complex, wherein a first nucleotide unit of the first multivalent molecule binds to the first DNA polymerase; and
b) binding a second universal nucleic acid primer, a second DNA polymerase, and the first multivalent molecule to a second portion of the same concatemer template molecule thereby forming a second binding complex, wherein a second nucleotide unit of the first multivalent molecule binds to the second DNA polymerase, wherein the first and second binding complexes which include the same multivalent molecule forms an avidity complex, wherein the first multivalent molecule comprises a core attached to multiple nucleotide arms and each nucleotide arm is attached to a nucleotide unit, and wherein the concatemer molecule comprises two or more tandem repeat sequences of a sequence of interest (110) and a universal primer binding site that binds the first and second universal nucleic acid primers.

13. The method of claim 10, wherein the method comprises:
a) binding a first universal nucleic acid primer, a first DNA polymerase, and a first multivalent molecule to a first portion of the concatemer molecules, thereby forming a first binding complex, wherein a first nucleotide unit of the first multivalent molecule binds to the first DNA polymerase; and
b) binding a second universal nucleic acid primer, a second DNA polymerase, and the first multivalent molecule to a second portion of the same concatemer template molecule thereby forming a second binding complex, wherein a second nucleotide unit of the first multivalent molecule binds to the second DNA polymerase, wherein the first and second binding complexes which include the same multivalent molecule forms an avidity complex, wherein the first multivalent molecule comprises a core attached to multiple nucleotide arms and each nucleotide arm is attached to a nucleotide unit, and wherein the concatemer molecule comprises two or more tandem repeat sequences of a sequence of interest (110) and a universal primer binding site that binds the first and second universal nucleic acid primers, and wherein the contacting is conducted under a condition suitable to inhibit polymerase-catalyzed incorporation of the bound first and second nucleotide units in the first and second binding complexes;
c) detecting the first and second binding complexes on the same concatemer template molecule, and identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

14. The method of claim 1, wherein nucleic acid sequence information is obtained for a longer nucleic acid sequence comprising a length of at least 500 bases.

15. The method of claim 1, wherein nucleic acid sequence information is obtained for a longer nucleic acid sequence comprising a length of at least 1,000 bases.

16. The method of claim 1, wherein nucleic acid sequence information is obtained for a longer nucleic acid sequence comprising a length from about 1,000 bases to about 40,000 bases.

17. The method of claim 1, wherein nucleic acid sequence information is obtained for a longer nucleic acid sequence comprising a length of up to about 35 kilobases.

18. The method of claim 1, wherein the nucleic acid sequence information is obtained from about 5,000 to about 25,000 independent groups of reads.

19. The method of claim 1, wherein a longer nucleic acid sequence resulting from the method is about two-fold longer than a nucleic acid sequence resulting from an alternate method for obtaining nucleic acid sequence information.

20. The method of claim 1, wherein the method provides about a two-fold increase in the amount of reads in comparison to an alternate method for obtaining nucleic acid sequence information.
